SEQUENCE LISTING (9 sequences)

SEQ ID NO: 1 - DNA, 7 bp, Artificial sequence
  Synthetic nucleotide
atggctg

SEQ ID NO: 2 - DNA, 7 bp, Artificial sequence
  Synthetic nucleotide
cttggtc

SEQ ID NO: 3 - DNA, 14 bp, Artificial sequence
  Synthetic nucleotide
attttgcttctctg

SEQ ID NO: 4 - DNA, 9 bp, Artificial sequence
  Synthetic nucleotide
aggattcgc

SEQ ID NO: 5 - DNA, 8 bp, Artificial sequence
  Synthetic nucleotide
ttgcttta

SEQ ID NO: 6 - DNA, 20 bp, Artificial sequence
  Synthetic nucleotide
aaaaaaaaaaaaaaaaaaaa

SEQ ID NO: 7 - DNA, 20 bp, Artificial sequence
  Synthetic nucleotide
gggggggggggggggggggg

SEQ ID NO: 8 - DNA, 20 bp, Artificial sequence
  Synthetic nucleotide
cccccccccccccccccccc

SEQ ID NO: 9 - DNA, 20 bp, Artificial sequence
  Synthetic nucleotide
tttttttttttttttttttt

(19) United States
(12) Patent Application Publication
(10) Pub. No.: US 2018/0088048 A1
(43) Pub. Date: Mar. 29, 2018

(54) DEVICES, METHODS, AND SYSTEMS RELATING TO SUPER RESOLUTION IMAGING

(71) Applicant: Northwestern University, Evanston, IL (US)

(72) Inventors: Biqin Dong, Evanston, IL (US); Janel L. Davis, Evanston, IL (US); Cheng Sun, Wilmette, IL (US); Hao F. Zhang, Deerfield, IL (US); Kieren J. Patel, Santa Monica, CA (US); Ben Urban, Evanston, IL (US); Vadim Backman, Chicago, IL (US); Luay Almassalha, Chicago, IL (US); Yolanda Stypula-Cyrus, Stoughton, WI (US); The-Quyen Nguyen, Evanston, IL (US)

(21) Appl. No.: 15/584,018

(22) Filed: May 1, 2017

Related U.S. Application Data
(60) Provisional application No. 62/329,856, filed on Apr. 29, 2016.
(60) Provisional application No. 62/329,859, filed on Apr. 29, 2016.
(60) Provisional application No. 62/329,865, filed on Apr. 29, 2016.
(60) Provisional application No. 62/329,867, filed on Apr. 29, 2016.
(60) Provisional application No. 62/329,868, filed on Apr. 29, 2016.
(60) Provisional application No. 62/329,871, filed on Apr. 29, 2016.

(51) Int. Cl. G01N 21/64; C12Q 1/6825; G01N 33/53

(52) U.S. Cl. CPC G01N 21/6428; G01N 21/6458; G01N 2021/6441; G01N 33/5302; C12Q 1/6825

(57) ABSTRACT

The devices, methods, and systems of the present disclosure provide for spectroscopic super-resolution microscopic imaging. In some examples, spectroscopic super-resolution microscopic imaging may be referred to or comprise spectroscopic photon localization microscopy (SPLM), a method which may employ the use of extrinsic labels or tags in a test sample suitable for imaging. In some examples spectroscopic super-resolution microscopic or spectroscopic photon localization microscopy (SPLM) may not employ extrinsic labels and be performed using the intrinsic contrast of the test sample or test sample material.

Generally, spectroscopic super-resolution microscopic imaging may comprise resolving one or more non-diffraction limited images of an area of a test sample by acquiring both localization information of a subset of molecules using microscopic methods known in the art, and simultaneously or substantially simultaneously, acquiring spectral data about the same or corresponding molecules in the subset. This method maybe useful to detect a variety of features in cellular material for the molecular characterization of cells and disease.

### RELATED APPLICATIONS

This patent arises from U.S. Provisional Patent Application Ser. No. 62/329,856, which was filed on Apr. 29, 2016, U.S. Provisional Patent Application Ser. No. 62/329,859, which was filed on Apr. 29, 2016, U.S. Provisional Patent Application Ser. No. 62/329,865, which was filed on Apr. 29, 2016, U.S. Provisional Patent Application Ser. No. 62/329,867, which was filed on Apr. 29, 2016, U.S. Provisional Patent Application Ser. No. 62/329,868, which was filed on Apr. 29, 2016, and U.S. Provisional Patent Application Ser. No. 62/329,871, which was filed on Apr. 29, 2016. U.S. Patent Application Ser. No. 62/329,856, U.S. Patent Application Ser. No. 62/329,859, U.S. Patent Application Ser. No. 62/329,865, U.S. Patent Application Ser. No. 62/329,867, U.S. Patent Application Ser. No. 62/329,868, and U.S. Patent Application Ser. No. 62/329,871 are hereby incorporated herein by reference in their entirety.

### STATEMENT REGARDING FEDERALLY SPONSORED RESEARCH FOR DEVELOPMENT

This invention was made with government support under CBET1066776 CBET1055379, DBI1353952, EEC1530734, and EFRI1240416 awarded by the National Science Foundation. The government has certain rights in the invention.

### BACKGROUND OF THE DISCLOSURE

While electron microscopy (EM) and scanning probe microscopy (SPM), are widely successful and commonly adopted methods for high resolution imaging of various materials, these methods are insufficient for non-invasive imaging of internal polymer structural information and embedded materials. While both these methods can provide information on the nanoscopic scale, they often require harsh sample preparation than may either damage or destroy the imaged sample. Advantageously, optical microscopes can non-invasively discern internal features and optical signatures of materials. For example, optical microscopy can be used to monitor internal single molecule distributions and locate defects inside of crystals. However, the spatial resolution of conventional optical imaging methods is fundamentally limited by optical diffraction, far below that of EM and SPM techniques. Therefore, there is need in the art to develop super-resolution optical imaging methods using a material's intrinsic physical and/or chemical properties, and/or through extrinsic labeling, that can offer unique advantages in the visualization and characterization of cellular genetic material, especially as relates to diagnostic and prognostic biomedical applications. In some cases, super resolution imaging with minimal damage or perturbation to the sample may be preferred.

The most recent advances in super-resolution optical imaging techniques, such as stochastic optical reconstruction microscopy (STORM), photoactivated localization microscopy (PALM), stimulated emission depletion (STED), and structured illumination microscopy (SIM), may extend the ability to study sub-diffraction-limited features that were previously thought to be unresolvable and have been applied to a myriad of applications including biological imaging, medical imaging for the diagnosis of disease, optimizing lithography techniques, directly observed catalytic effects of metallic nanoparticles on a molecular scale, and tracked single polymer molecules.

The vast majority of these super-resolution technologies rely on extrinsic contrast agents. Extrinsic agents can have multiple weaknesses, including (1) they require additional labeling processes, (2) they modify physical properties of the test sample material, and (3) they introduce inaccurate spatial localization caused by the physical dimension of the tagged fluorescent and linker molecule (4), due to spectral overlap, a limited number of labels may be resolved or may confound imaging signals leading to inaccuracy. The combination of these weaknesses reduces the appeal of extrinsic fluorescent contrast agents with traditional imaging methods. There is need in the art for improved super-resolution methods of imaging cellular genetic material that do not require extrinsic labels, and/or that are able to better resolve samples with extrinsic labeling for improved diagnostic and/or prognostic imaging.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIGS. 1a-4e illustrate example image and molecule characteristics.

FIG. 5 illustrates an example Jablonski diagram of a three level system

FIG. 6 illustrates example recovery data.

FIGS. 7a-c illustrate example Nyquist resolution analysis.

FIGS. 8a-j illustrate example spectroscopic photon localization microscopy (SPLM) systems and associated principles.

FIGS. 9a-11f illustrate example images, data analysis, and results.

FIG. 12 illustrates an example flow diagram of a method to sequence nucleic acids and/or polymers by fingerprinting.

FIG. 13 illustrates an example analysis of an unknown sequence of nucleic acid or polymer.

FIG. 14 illustrates an example of sequencing by degradation using SPLM.

FIGS. 15 and 16 illustrate examples of sequencing by synthesis.

FIGS. 17-22b illustrate examples of analyte analysis.

FIG. 23 illustrates a flow diagram of an example method to calculate a dissociation constant or probe enzyme activity.

FIG. 24 illustrates an example droplet cell sorting system with SPLM.

FIG. 25 illustrates an example cell.

FIG. 26 illustrates a captured cell in a droplet for SPLM imaging.

FIG. 27 illustrates an apparatus for cell analysis.

FIG. 28 illustrates an example cell sorting based on membrane markers.

FIG. 29 illustrates an example system for sorting based on localization and spectral profile.

FIG. 30 illustrates another example apparatus for cell analysis.

FIG. 31 illustrates a flow diagram of an example method for SPLM resolution and cell analysis.

FIGS. 32-35 illustrate imaging labeling examples.

FIGS. 36-37 illustrate flow diagrams of example methods for imaging label and analysis.

FIG. 38 depicts example spectral profiles reflecting differences in spectral curve shape and size.

FIGS. 39a-b show an example of constructing an indexed library and immobilizing complexes on a substrate having a plurality of imaging label target molecule complexes.

FIGS. 40a-b illustrate an example methodology to image imaging label—target molecule complexes to count a number of target molecules in a sample.

FIG. 41 shows another example substrate with imaging labels and target molecules immobilized to a substrate for analysis.

FIGS. 42a-45 illustrate example systems and methods for pathogen characteristic analysis.

FIGS. 46-47 illustrate computing devices, systems, and/or platforms can be used in connection with examples disclosed and described herein.

The novel features of a device of this disclosure are set forth with particularity in the appended claims. A better understanding of the features and advantages of this disclosure will be obtained by reference to the following detailed description that sets forth illustrative examples, in which the principles of a device of this disclosure are utilized, and the accompanying drawings of which:

The following detailed description of certain examples of the present invention will be better understood when read in conjunction with the appended drawings. For the purpose of illustrating the invention, certain examples are shown in the drawings. It should be understood, however, that the present invention is not limited to the arrangements and instrumentality shown in the attached drawings.

### DETAILED DESCRIPTION OF THE DISCLOSURE

### I. General Overview

Many processes are characterized or regulated by the absolute or relative amounts of a plurality of items. For example, in biology, the level of expression of particular genes or groups of genes or the number of copies of chromosomal regions can be used to characterize the status of a cell or tissue. Analog methods such as microarray hybridization methods and real-time PCR are alternatives, but digital readout methods such as those disclosed herein have advantages over analog methods. Methods for estimating the abundance or relative abundance of genetic material having increased accuracy of counting would be beneficial.

The availability of convenient and efficient methods for the accurate identification of genetic variation and expression patterns among large sets of genes may be applied to understanding the relationship between an organism's genetic make-up and the state of its health or disease, Collins et al, Science, 282: 682-689 (1998). In this regard, techniques have been developed for the analysis of large populations of polynucleotides based either on specific hybridization of probes to microarrays, e.g. Lockhart et al. Hacia et al, Nature Genetics, 21: 4247 (1999), or on the counting of tags or signatures of DNA fragments, e.g. Velculescu et al, Science, 270: 484487 (1995); Brenner et al, Nature Biotechnology, 18: 630-634 (2000). These techniques have been used in discovery research to identify subsets of genes that have coordinated patterns of expression under a variety of circumstances or that are correlated with, and predictive of events, of interest, such as toxicity, drug responsiveness, risk of relapse, and the like, e.g. Golub et al, Science, 286: 531-537 (1999); Alizadeh et al, Nature, 403: 503-511 (2000); Perou et al, Nature, 406: 747-752 (2000); Shipp et al, Nature Medicine, 8: 68-74 (2002); Hakak et al, Proc. Natl. Acad. Sci., 98: 47454751 (2001); Thomas et al, Mol. Pharmacol., 60: 1189-1194 (2001); De Primo et al, BMC Cancer 2003, 3:3; and the like. Not infrequently the subset of genes found to be relevant has a size in the range of from ten or under to a few hundred.

In addition to gene expression, techniques have also been developed to measure genome-wide variation in gene copy number. For example, in the field of oncology, there is interest in measuring genome-wide copy number variation of local regions that characterize many cancers and that may have diagnostic or prognostic implications. For a review see Zhang et al. Annu. Rev. Genomics Hum. Genet. 2009. 10:451-81.

While such hybridization-based techniques offer the advantages of scale and the capability of detecting a wide range of gene expression or copy number levels, such measurements may be subject to variability relating to probe hybridization differences and cross-reactivity, element-to-element differences within microarrays, and microarray-to-microarray differences, Audic and Clayerie, Genomic Res., 7: 986-995 (1997); Wittes and Friedman, J. Natl. Cancer Inst. 91: 400-401 (1999).

On the other hand, techniques that provide digital representations of abundance, such as SAGE (Velculescu et al, cited above) or MPSS (Brenner et al, cited above), are statistically more robust; they do not require repetition or standardization of counting experiments as counting statistics are well-modeled by the Poisson distribution, and the precision and accuracy of relative abundance measurements may be increased by increasing the size of the sample of tags or signatures counted, e.g. Audic and Clayerie (cited above).

Both digital and non-digital hybridization-based assays have been implemented using oligonucleotide tags that are hybridized to their complements, typically as part of a detection or signal generation schemes that may include solid phase supports, such as microarrays, microbeads, or the like, e.g. Brenner et al, Proc. Natl. Acad. Sci., 97: 1665-1670 (2000); Church et al, Science, 240: 185-188 (1988); Chee, Nucleic Acids Research, 19: 3301-3305 (1991); Shoemaker et al., Nature Genetics, 14: 450456 (1996); Wallace, U.S. Pat. No. 5,981,179; Gerry et al, J. Mol. Biol., 292: 251-262 (1999); Fan et al., Genome Research, 10: 853-860 (2000); Ye et al., Human Mutation, 17: 305-316 (2001); and the like. Bacterial transcript imaging by hybridization of total RNA to nucleic acid arrays may be conducted as described in Saizieu et al., Nature Biotechnology, 16:45-48 (1998). Accessing genetic information using high density DNA arrays is further described in Chee et al., Science 274:610-614 (1996). Tagging approaches have also been used in combination with next-generation sequencing methods, see for example, Smith et al. NAR (May 11, 2010), 1-7.

A common feature among all of these approaches is a one-to-one correspondence between probe sequences and oligonucleotide tag sequences. That is, the oligonucleotide tags have been employed as probe surrogates for their favorable hybridizations properties, particularly under multiplex assay conditions.

Determining small numbers of biological molecules and their changes is essential when unraveling mechanisms of cellular response, differentiation or signal transduction, and in performing a wide variety of clinical measurements. Although many analytical methods have been developed to measure the relative abundance of different molecules through sampling (e.g., microarrays and sequencing), few techniques are available to determine the absolute number of molecules in a sample. This can be an important goal, for example in single cell measurements of copy number or stochastic gene expression, and is especially challenging when the number of molecules of interest is low in a background of many other species. As an example, measuring the relative copy number or expression level of a gene across a wide number of genes can currently be performed using PCR, hybridization to a microarray or by direct sequence counting. PCR and microarray analysis rely on the specificity of hybridization to identify the target of interest for amplification or capture respectively, then yield an analog signal proportional to the original number of molecules. A major advantage of these approaches is in the use of hybridization to isolate the specific molecules of interest within the background of many other molecules, generating specificity for the readout or detection step. The disadvantage is that the readout signal to noise is proportional to all molecules (specific and non-specific) specified by selective amplification or hybridization. The situation is reversed for sequence counting. No intended sequence specificity is imposed in the sequence capture step, and all molecules are sequenced. The major advantage is that the detection step simply yields a digital list of those sequences found, and since there is no specificity in the isolation step, all sequences must be analyzed at a sufficient statistical depth in order to learn about a specific sequence. Although very major technical advances in sequencing speed and throughput have occurred, the statistical requirements imposed to accurately measure small changes in concentration of a specific gene within the background of many other sequences requires measuring many sequences that don't matter to find the ones that do matter. Each of these techniques, PCR, array hybridization and sequence counting is a comparative technique in that they primarily measure relative abundance, and do not typically yield an absolute number of molecules in a solution. A method of absolute counting of nucleic acids is digital PCR (B. Vogelstein, K. W Kinzler, Proc Natl Acad Sci USA 96, 9236 (Aug. 3, 1999)), where solutions are progressively diluted into individual compartments until there is an average probability of one molecule per two wells, then detected by PCR. Although digital PCR can be used as a measure of absolute abundance, the dilutions must be customized for each type of molecule, and thus in practice is generally limited to the analysis of a small number of different molecules.

High-sensitivity single molecule digital counting by optical labeling of a collection of molecules with unique spectral profiles is disclosed. Each copy of a molecule randomly chooses from a non-depleting reservoir of imaging labels, wherein each label has a tag with a unique light emitting spectral profile. The uniqueness of each labeled molecule is determined by the statistics of random choice, and depends on the number of copies of identical molecules in the collection compared to the diversity of labels. The size of the resulting set of labeled molecules is determined by the nature of the labeling process, and analysis reveals the original number of molecules. When the number of copies of a molecule to the diversity of labels is low, the labeled molecules are highly unique, and the counting efficiency is high. The conceptual framework for stochastic mapping of a variety of molecule types is developed and the utility of the methods. The labeled fragments for a target molecule of choice are detected with high specificity using a system similar to microarray readout system, wherein imaging label-targe molecules may be immobilized to a substrate for imaging of individual bound complexes.

Methods are disclosed herein for optical counting of individual molecules of one or more target molecules. In preferred embodiments the targets are nucleic acids, but may be a variety of biological or non-biological elements. Targets are labeled so that individual occurrences of the same target are marked by attachment of a different imaging label to difference occurrences. The attachment of the label confers a separate, determinable identity to each occurrence of targets that may otherwise be indistinguishable. Preferably the labels are different light emitting tags that produce a unique spectral profile when illuminated by excitation light, and wherein each tag marks each target molecule occurrence uniquely. The resulting modified target molecule comprises the target sequence and the imaging label (which may be referred to herein as tag, counter, label, or marker). The junction of the target and identifier forms a uniquely detectable mechanism for counting the occurrence of that copy of the target. The attachment of the identifier to each occurrence of the target is a random sampling event. Each occurrence of target could choose any of the labels. Each identifier is present in multiple copies so selection of one copy does not remove that identifier sequence from the pool of identifiers so it is possible that the same identifier will be selected twice. The probability of that depends on the number of target occurrences relative to the number of different identifier sequences.

For a given target, all resulting products will contain the same target portion and in some cases but each will contain a different tag of unique spectral profile that may be able to be distinguished between tags of a similar spectral profiles for the same target molecule. (T1S1, T1S2, . . . T1SN where N is the number of different occurrences of target1, “T1” and S is the associated spectral signature of the tag associated with the imaging label, L1, L2 . . . LN). In preferred aspects the occurrences are detected by hybridization. In some aspects the methods and systems include a probe array comprising features, wherein each feature has a different combination of target sequence with identifiers, 1 to N wherein N is the number of unique identifiers in the pool of identifiers. The array has N features for each target, so if there are 8 targets to be analyzed there are 8 times N features on the array to interrogate the 8 targets. Generally, individual spectral signatures and localization of imaging label—target molecules are imaged and resolved by spectroscopic photon localization microscopy (SPLM) as described herein. In some cases, SPLM may be also used to determine the sequence of the target molecule or imaging label by comparison of the spectral pattern produced by the imaging label—target molecule complex and a known set of indexed library of spectral profiles, wherein each profile is associated with a known sequence. In some cases, no extrinisic labeling is required and the probe of the imaging label is the same as the light emitting tag. In this case, excitation light may be used to induce oligonucleotides, or hybrid oligo complexes to emit light which can be imaged using SPLM to detect a unique spectral profile derived from a unique nucleotide sequence.

The devices, methods, and systems of the present disclosure provide for spectroscopic super-resolution microscopic imaging for the determination of the sequence molecule count of target molecules in a sample, such as that derived from a single cell. In some examples, spectroscopic super-resolution microscopic imaging may be referred to or comprise spectroscopic photon localization microscopy (SPLM), a method which may employ the use of extrinsic labels or tags in a test sample suitable for imaging. In some examples spectroscopic super-resolution microscopic or spectroscopic photon localization microscopy (SPLM) may not employ extrinsic labels and be performed using the intrinsic contrast of the test sample or test sample material.

Generally, spectroscopic super-resolution microscopic imaging may comprise resolving one or more non-diffraction limited images of an area of a test sample by acquiring both localization information of a subset of molecules or moieties using microscopic methods known in the art, and simultaneously or substantially simultaneously, acquiring spectral data about the same or corresponding molecules in the subset.

Together, both microscopic localization and spectral information can be used to generate one or more non-diffraction limited images. In some examples, the signal used for acquiring microscopic localization and spectral information may be derived from an extrinsic label applied to one or more molecules in the test sample. In some examples, the signal used for acquiring microscopic localization and spectral information may be derived from the intrinsic contrast or inherent chemical and physical properties (e.g. electronic configuration) of the test sample or test sample material.

### II. General Methods for Spectroscopic Super-Resolution Microscopic Imaging

### A. Terminology and Spectroscopic Super-Resolution Microscopic Imaging Methods

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain examples, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The term “spectroscopic super-resolution microscopic imaging” described herein, generally refers to any general optical imaging method that uses both microscopic single molecule localization of molecules in a test sample and spectroscopic information about those molecules in a test sample to generate one or more non-diffraction limited images. In some examples single molecule localization of molecules and spectroscopic information is captured to resolve one or more non-diffraction limited images simultaneously.

The term “activating” may refer to any change in the electronic state of a molecule. In some examples, this may refer to excitation of the molecule to fluoresce. In some examples this may refer to Raman scattering.

“Detector”: As used herein, the term “detector” includes any detector of electromagnetic radiation including, but not limited to, charge-coupled device (CCD) camera, photomultiplier tubes, photodiodes, and avalanche photodiodes.

“Sensor”: As used herein, the term “sensor” includes any sensor of electromagnetic radiation including, but not limited to, CCD camera, photomultiplier tubes, photodiodes, and avalanche photodiodes, unless otherwise evident from the context.

“Image”: The term “image”, as used herein, is understood to mean a visual display or any data representation that may be interpreted for visual display. For example, a three-dimensional image may include a dataset of values of a given quantity that varies in three spatial dimensions. A three-dimensional image (e.g., a three-dimensional data representation) may be displayed in two-dimensions (e.g., on a two-dimensional screen, or on a two-dimensional printout). The term “image” may refer, for example, to an optical image.

“Substantially”: As used herein, the term “substantially”, and grammatical equivalents, refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result.

In the present disclosure, a “test sample” may indicate any sample, object, or subject suitable for imaging.

### B. Spectroscopic Super-resolution Microscopic Imaging System Configurations

A spectroscopic super-resolution microscopic system for data collection may be configured in a variety of ways, generally incorporating optical components capable of simultaneously performing single molecule microscopic localization and spectroscopy on a test sample. In some examples, the devices, methods, and systems of the disclosure may us any suitable imager including but not limited to a charged coupled device (CCD), electron multiplying charged coupled device (EMCCD), camera, and complementary metal-oxide-semiconductor (CMOS) imager.

In some examples, the devices, methods, and systems of the disclosure may us any suitable spectral filtering element including but not limited to a dispersive element, transmission grating, grating, band pass filter or prism.

The devices, methods, and systems of the present disclosure may use any light source suitable for spectroscopic super-resolution microscopic imaging, including but not limited to a laser, laser diode, visible light source, ultraviolet light source or infrared light source, superluminescent diodes, continuous wave lasers or ultrashort pulsed lasers.

Generally, the wavelength range of one or more beams of light may range from about 500 nm to about 620 nm, for example. In some examples, the wavelength may range between 200 nm to 600 nm. In some examples, the wavelength may range between 300 to 900 nm. In some examples, the wavelength may range between 500 nm to 1200 nm. In some examples, the wavelength may range between 500 nm to 800 nm. In some examples, the wavelength range of the one or more beams of light may have wavelengths at or around 500 nm, 510 nm, 520 nm, 530 nm, 540 nm, 550 nm, 560 nm, 570 nm, 580 nm, 590 nm, 600 nm, 610 nm, and 620 nm. Generally, the wavelength range of the one or more beams of light may range from 200 nm to 1500 nm. In some examples, the wavelength range of the one or more beams of light may range from 200 nm to 1500 nm. The wavelength range of the one or more beams of light may range from 300 nm to 1500 nm. The wavelength range of the one or more beams of light may range from 400 nm to 1500 nm. The wavelength range of the one or more beams of light may range from 500 nm to 1500 nm. The wavelength range of the one or more beams of light may range from 600 nm to 1500 nm. The wavelength range of the one or more beams of light may range from 700 nm to 1500 nm. The wavelength range of the one or more beams of light may range from 800 nm to 1500 nm. The wavelength range of the one or more beams of light may range from 900 nm to 1500 nm. The wavelength range of the one or more beams of light may range from 1000 nm to 1500 nm. The wavelength range of the one or more beams of light may range from 1100 nm to 1500 nm. The wavelength range of the one or more beams of light may range from 1200 nm to 1500 nm. The wavelength range of the one or more beams of light may range from 1300 nm to 1500 nm. The wavelength range of the one or more beams of light may range from 1300 nm to 1500 nm. In some examples, spectroscopic super-resolution microscopic imaging devices, methods, and systems of the present disclosure include two or more beams of light with wavelengths in the visible light spectrum or the near infrared (NIR) light spectrum. In some examples, spectroscopic super-resolution microscopic imaging includes beams of light with wavelengths in the visible light spectrum, UV or the NIR spectrum. Those of skill in the art will appreciate that the wavelength of light may fall within any range bounded by any of these values (e.g., from about 200 nm beam to about 1500 nm).

In some examples, spectroscopic super-resolution microscopic imaging may include multi-band scanning. In some examples a band may include one or more wavelength ranges containing continuous wavelengths of light within a bounded range. In some examples a band may include one or more wavelength ranges containing continuous group of wavelengths of light with an upper limit of wavelengths and a lower limit of wavelengths. In some examples, the bounded ranges within a band may include the wavelength ranges described herein. In some examples spectroscopic super-resolution microscopic imaging may include bands that overlap. In some examples, spectroscopic super-resolution microscopic imaging may include bands that are substantially separated. In some examples, bands may partially overlap. In some examples, spectroscopic super-resolution microscopic may include one or more bands ranging from 1 band to 100 bands. In some examples, the number of bands may include 1-5 bands. In some the number of bands may include 5-10 bands. In some examples, the number of bands may include 10-50 bands. In some the number of bands may include 25-75 bands. In some examples, the number of bands may include 25-100 bands. Those of skill in the art will appreciate that the number of bands of light may fall within any range bounded by any of these values (e.g., from about 1 band to about 100 bands).

In some cases, the frequency of light of one or more beams of light, or bands used in spectroscopic super-resolution microscopic imaging may be chosen based on the absorption-emission bands known for a test sample. In some examples, for example, a wavelength or wavelengths of light may be chosen such that those wavelengths are within the primary absorption-emission bands known or thought to be known for a particular test sample.

Further, the devices, methods, and systems of the disclosure may allow for various power requirements or laser fluences to generate spectroscopic super-resolution microscopic images. In some examples, a spectroscopic super-resolution microscopic imaging device is configured to illuminate a test sample with a light source with a fluence from 0.01 kW/cm² to 100 kW/cm². In some examples, a spectroscopic super-resolution microscopic imaging device is configured to illuminate a test sample with a light source fluence of about 5 kW/cm². In some examples, a spectroscopic super-resolution microscopic imaging device is configured to illuminate a test sample with a light source with a fluence from 0.01 kW/cm² to 0.05 kW/cm². In some examples, a spectroscopic super-resolution microscopic imaging device is configured to illuminate a test sample with a light source with a fluence from 0.1 kW/cm² to 0.5 kW/cm². In some examples, a spectroscopic super-resolution microscopic imaging device is configured to illuminate a test sample with a light source with a fluence from 0.02 kW/cm² to 0.8 kW/cm². In some examples, a spectroscopic super-resolution microscopic imaging device is configured to illuminate a test sample with a light source with a fluence from 0.2 kW/cm² to 0.6 kW/cm². In some examples, a spectroscopic super-resolution microscopic imaging device is configured to illuminate a test sample with a light source with a fluence from 0.5 kW/cm² to 1.0 kW/cm². In some examples, a spectroscopic super-resolution microscopic imaging device is configured to illuminate a test sample with a light source fluence of about 2 kW/cm²-8 kW/cm². In some examples, a spectroscopic super-resolution microscopic imaging device is configured to illuminate a test sample with a light source fluence of about 1 kW/cm²-10 kW/cm². In some examples, a spectroscopic super-resolution microscopic imaging device is configured to illuminate a test sample with a light source of about 2 kW/cm²-9 kW/cm². In some examples, a spectroscopic super-resolution microscopic imaging device is configured to illuminate a test sample with a light source with a fluence ranging from 3 kW/cm² to 6 kW/cm². In some examples, a spectroscopic super-resolution microscopic device is configured to illuminate a test sample with a light source with a fluence ranging from 2 kW/cm² to 20 kW/cm². In some examples, a spectroscopic super-resolution microscopic imaging device is configured to illuminate a test sample with a light source with a fluence ranging from 5 kW/cm² to 50 kW/cm². In some examples, a spectroscopic super-resolution microscopic imaging device is configured to illuminate a test sample with a light source with a fluence ranging from 10 kW/cm² to 75 kW/cm². In some examples, a spectroscopic super-resolution microscopic imaging device is configured to illuminate a test sample with a light source with a fluence ranging from 50 kW/cm² to 100 kW/cm². In some examples, a spectroscopic super-resolution microscopic imaging device is configured to illuminate a test sample with a light source with a power ranging from 75 kW/cm² to 100 kW/cm². In some examples, a spectroscopic super-resolution microscopic imaging device is configured to illuminate a test sample with a light source with a fluence ranging from 1 kW/cm² to 40 kW/cm². Those of skill in the art will appreciate that light source fluence may fall within any range bounded by any of these values (e.g. from about 0.01 kW/cm² to about 100 kW/cm²).

### C. Light Emitting Molecules, Extrinsic Labels and Intrinsic Contrast

i. Light Emitting Molecules

The devices, methods, and systems of the disclosure provide for the capturing of one or more light emitting molecules. In some examples, a light-emitting molecules may be any molecule that may emit a photon at any wavelength. In some examples, a light-emitting molecule may be fluorophore. In some cases, a light-emitting molecule emits a photon after illumination and excitation with one or more wavelengths of light.

ii. Extrinsic Labels

Extrinsic labels may be molecules or specific probes that to emit signals detected during spectroscopic super-resolution microscopic. In some examples, an extrinsic label may be covalently bound to a molecule, thus making the entire molecular entity a light-emitting molecule. In some examples, an extrinsic label may be one or more non-covalently bound to a molecule, also making the entire molecular entity a light-emitting molecule. Any labels suitable for generating such signals can be used in the present invention. In some examples, the signals are generated by fluorophores. Fluorescent labeling, e.g., the process of covalently attaching a fluorophore to a probe that binds to another molecule or cellular constituent (such as a protein or nucleic acid), is generally accomplished using a reactive derivative of the fluorophore that selectively binds to a functional group contained in the test sample molecule. The molecule may also be bound non covalently though the use of antibodies. In some examples, the fluorophore in a quantum dot. In some examples, example probes to which the labels are attached include but are not limited to antibodies, proteins, amino acids and peptides. Common reactive groups include amine reactive isothiocyanate derivatives such as FITC and TRITC (derivatives of fluorescein and rhodamine), amine reactive succinimidyl esters such as NHS-fluorescein, and sulfhydryl reactive maleimide activated fluors such as fluorescein-5-maleimide, etc.

In some examples, labels of the present disclosure include one or more fluorescent dyes, including but not limited to fluorescein, rhodamine, Alexa Fluors, DyLight fluors, ATTO Dyes, or any analogs or derivatives thereof. fluorescent tag, fluorescent protein, fluorophore, fluorescent probe, quantum dot, fluorescence resonance energy transfer probe, and diode laser excitable probe used with any dyes or other labels as described herein.

In some examples, labels of the present disclosure include but are not limited to fluorescein and chemical derivatives of fluorescein; Eosin; Carboxyfluorescein; Fluorescein isothiocyanate (FITC); Fluorescein amidite (FAM); Erythrosine; Rose Bengal; fluorescein secreted from the bacterium Pseudomonas aeruginosa; Methylene blue; Laser dyes; Rhodamine dyes (e.g., Rhodamine, Rhodamine 6G, Rhodamine B, Rhodamine 123, Auramine O, Sulforhodamine 101, Sulforhodamine B, and Texas Red).

In some examples, labels of the present disclosure include but are not limited to ATTO dyes; Acridine dyes (e.g., Acridine orange, Acridine yellow); Alexa Fluor; 7-Amino actinomycin D; 8-Anilinonaphthalene-1-sulfonate; Auramine-rhodamine stain; Benzanthrone; 5,12-Bis(phenylethynyl)naphthacene; 9,10-Bis(phenylethynyl)anthracene; Blacklight paint; Brainbow; Calcein; Carboxyfluorescein; Carboxyfluorescein diacetate succinimidyl ester; Carboxyfluorescein succinimidyl ester; 1-Chloro-9,10-bis(phenylethynyl)anthracene; 2-Chloro-9,10-bis(phenylethynyl)anthracene; 2-Chloro-9,10-diphenylanthracene; Coumarin; Cyanine dyes (e.g., Cyanine such as Cy3 and Cy5, DiOC6, SYBR Green I); DAPI, Dark quencher, DyLight Fluor, Fluo-4, FluoProbes; Fluorone dyes (e.g., Calcein, Carboxyfluorescein, Carboxyfluorescein diacetate succinimidyl ester, Carboxyfluorescein succinimidyl ester, Eosin, Eosin B, Eosin Y, Erythrosine, Fluorescein, Fluorescein isothiocyanate, Fluorescein amidite, Indian yellow, Merbromin); Fluoro-Jade stain; Fura-2; Fura-2-acetoxymethyl ester; Green fluorescent protein, Hoechst stain, Indian yellow, Indo-1, Lucifer yellow, Luciferin, Merocyanine, Optical brightener, Oxazin dyes (e.g., Cresyl violet, Nile blue, Nile red); Perylene; Phenanthridine dyes (Ethidium bromide and Propidium iodide); Phloxine, Phycobilin, Phycoerythrin, Phycoerythrobilin, Pyranine, Rhodamine, Rhodamine 123, Rhodamine 6G, RiboGreen, RoGFP, Rubrene, SYBR Green I, (E)-Stilbene, (Z)-Stilbene, Sulforhodamine 101, Sulforhodamine B, Synapto-pHluorin, Tetraphenyl butadiene, Tetrasodium tris(bathophenanthroline disulfonate)ruthenium(II), Texas Red, TSQ, Umbelliferone, or Yellow fluorescent protein.

In some examples, labels of the present disclosure include but are not limited to the Alexa Fluor family of fluorescent dyes (Molecular Probes, Oregon). Alexa Fluor dyes are typically used as cell and tissue labels in fluorescence microscopy and cell biology. The excitation and emission spectra of the Alexa Fluor series cover the visible spectrum and extends into the infrared. The individual members of the family are numbered according roughly to their excitation maxima (in nm). Alexa Fluor dyes are synthesized through sulfonation of coumarin, rhodamine, xanthene (such as fluorescein), and cyanine dyes. Sulfonation makes Alexa Fluor dyes negatively charged and hydrophilic. Alexa Fluor dyes are generally more stable, brighter, and less pH-sensitive than common dyes (e.g. fluorescein, rhodamine) of comparable excitation and emission, and to some extent the newer cyanine series. Example Alexa Fluor dyes include but are not limited to Alexa-350, Alexa-405, Alexa-430, Alexa-488, Alexa-500, Alexa-514, Alexa-532, Alexa-546, Alexa-555, Alexa-568, Alexa-594, Alexa-610, Alexa-633, Alexa-647, Alexa-660, Alexa-680, Alexa-700, or Alexa-750.

In some examples, labels of the present disclosure include one or more members of the DyLight Fluor family of fluorescent dyes (Dyomics and Thermo Fisher Scientific). Exemplary DyLight Fluor family dyes include but are not limited to DyLight-350, DyLight-405, DyLight-488, DyLight-549, DyLight-594, DyLight-633, DyLight-649, DyLight-680, DyLight-750, or DyLight-800.

In some examples, when pairs of dyes are used (as described in greater detail herein below) the activator choices include Alexa405, 488, 532 and 568, and the emitter choices include Cy5, Cy5.5, Cy7, and 7.5. Using these particular choices, because they can be mixed and matched to give functional dye pairs, there are 16 possible pairs (4×4) in all.

In some examples, a light-emitting molecule may be stochastically activated. In some cases, stochastically activated may comprise photoswitching, or stochastic emission of light (“blinking”). In some examples, a switchable entity may be used. Non-limiting examples of switchable entities are discussed in International Patent Application No. PCT/US2007/017618, filed Aug. 7, 2007, entitled “Sub-Diffraction Limit Image Resolution and Other Imaging Techniques,” published as Int. Pat. Apl. Pub. No. WO 2008/091296 on Jul. 31, 2008, incorporated herein by reference. As a non-limiting example of a switchable entity, Cy5 can be switched between a fluorescent and a dark state in a controlled and reversible manner by light of different wavelengths, e.g., 633 nm or 657 nm red light can switch or deactivate Cy5 to a stable dark state, while 532 nm green light can switch or activate the Cy5 back to the fluorescent state. Other non-limiting examples of a switchable entity including photoactivatable or photoswitchable fluorescent proteins, or photoactivatable or photoswitchable inorganic particles, e.g., as discussed herein. In some examples, the entity can be reversibly switched between the two or more states, e.g., upon exposure to the proper stimuli. For example, a first stimuli (e.g., a first wavelength of light) may be used to activate the switchable entity, while a second stimuli (e.g., a second wavelength of light) may be used to deactivate the switchable entity, for instance, to a non-emitting state. Any suitable method may be used to activate the entity. For example, in one example, incident light of a suitable wavelength may be used to activate the entity to emit light, e.g., the entity is photoswitchable. Thus, the photoswitchable entity can be switched between different light-emitting or non-emitting states by incident light, e.g., of different wavelengths. The light may be monochromatic (e.g., produced using a laser) or polychromatic. In another example, the entity may be activated upon stimulation by electric field and/or magnetic field. In other examples, the entity may be activated upon exposure to a suitable chemical environment, e.g., by adjusting the pH, or inducing a reversible chemical reaction involving the entity, etc. Similarly, any suitable method may be used to deactivate the entity, and the methods of activating and deactivating the entity need not be the same. For instance, the entity may be deactivated upon exposure to incident light of a suitable wavelength, or the entity may be deactivated by waiting a sufficient time.

In some examples, the entities may be independently switchable, e.g., the first entity may be activated to emit light without activating a second entity. For example, if the entities are different, the methods of activating each of the first and second entities may be different (e.g., the entities may each be activated using incident light of different wavelengths). As another non-limiting example, incident light having a sufficiently weak intensity may be applied to the first and second entities such that only a subset or fraction of the entities within the incident light are activated, e.g., on a stochastic or random basis. Specific intensities for activation can be determined by those of ordinary skill in the art using no more than routine skill. By appropriately choosing the intensity of the incident light, the first entity may be activated without activating the second entity.

The second entity may be activated to emit light, and, optionally, the first entity may be deactivated prior to activating the second entity. The second entity may be activated by any suitable technique, as described herein, for instance, by application of suitable incident light.

In some examples, incident light having a sufficiently weak intensity may be applied to a plurality of entities such that only a subset or fraction of the entities within the incident light are activated, e.g., on a stochastic or random basis. The amount of activation may be any suitable fraction or subset, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the entities may be activated, depending on the application. For example, by appropriately choosing the intensity of the incident light, a sparse subset of the entities may be activated such that at least some of them are optically resolvable from each other and their positions can be determined. Iterative activation cycles may allow the positions of all of the entities, or a substantial fraction of the entities, to be determined. In some cases, an image with non-diffraction limit resolution can be constructed using this information.

iii. Intrinsic Contrast

In some examples, an extrinsic label may not be applied to test sample. In some examples, light-emitting molecules in an area of a test sample are not subject to an extrinsic label. In some examples, molecules within the test sample my emit photons, or fluoresce without the need of an extrinsic label. For example, certain polymers may have suitable absorption-emission bands such that individual molecules, or subunits within the polymer emit light when excited by a suitable wavelength of light. Generally, detection of light emission without the use of extrinsic labels may be referred to as intrinsic contrast.

In some examples, light emitting may be the result of any perturbation or change in the electronic state of the test sample. In some cases, and as described herein, a perturbation or change in the electronic state of test sample might result in fluorescence. In some examples, any perturbation or change in the electronic state of the test sample may result in Raman scattering. Generally, the devices, methods, and systems of the disclosure provide for use of signals from any light-emitting molecules, including but not limited to Raman spectroscopy, optical fluorescence microscopy, infrared spectroscopy, ultraviolet spectroscopy, laser microscopy and confocal microscopy.

### D. Single Molecule Localization

The devices, methods, and systems of the disclosure provide for capturing one or more images of the light and localizing the light-emitting particles using one or more single molecule microscopic methods. In some examples, a spectral filtering element, such as a diffraction grating or band pass filter may allow the generation of zero-order and first-order images for further analysis. Zero-order images may be used to determine the probabilistic locations of the light-emitting molecules from their localized point spread functions.

Generally, single molecule localization comprises selecting emission spots in a desired wavelength range corresponding to light-emitting molecules. In some examples, there may be a single emission wavelength range. In alternative examples, there may be two or more wavelength ranges. The method may include only identifying and processing in focus spots, whether or not they are centered on expected illumination positions. In particular, by suitable selection of in focus spots, significant improvements in axial resolution can be achieved. Emission spots may be identified using any suitable localization method including but not limited to those adapted for use with stochastic imaging approaches such as stochastic optical reconstruction microscopy, spectral precision distance microscopy (SPDM), spectral precision distance microscopy with physically modifiable fluorophores (SPDMphymod), photo activated localization microscopy (PALM), photo-activation localization microscopy (FPALM), photon localization microscopy (PLM), direct stochastical optical reconstruction microscopy (dSTORM), super-resolution optical fluctuation imaging (SOFI), and 3D light microscopical nanosizing microscopy (LIMON). In some examples, single molecule localization methods may also comprise methods derived for particle tracking.

The centroid of each identified spot may be located using any suitable method including but not limited to those used for particle localization and tracking and stochastic imaging approaches such as PALM/STORM and SOFI and other described herein. In some examples of each identified spot may be determined by using nonlinear curve fitting of a symmetric Gaussian function with a fixed standard deviation. The standard deviation value may be fixed based on estimation or may be fixed based on an average value determined from identified spots. Enhancing each image or sub images may be carried out by any suitable technique including but not limited to those developed for PALM, STORM and SOFI and other described herein. In some examples, enhancement is carried out using a Gaussian mask. The Gaussian mask may have a fixed or user defined standard deviation. Enhancement may additionally or alternatively include scaling the sub image. In some examples, a scale factor of the order 2 may be applied to the sub image.

### E. Spectroscopic Methods and Analysis

i. Spectral Unmixing

The devices, methods, and systems of the disclosure provide for one or more spectroscopic analyses of the corresponding emission spectrum of the one or more localized activated light-emitting molecules. As described herein, the emission spectra for each light-emitting molecule may be captured with a spectrometer via methods known in the art related to Raman spectroscopy, optical fluorescence microscopy, infrared spectroscopy, ultraviolet spectroscopy, laser microscopy and confocal microscopy.

Generally, a first-order image, generated through the use of a spectral filtering element, such as a diffraction element or prism, allows individual spectra to be captured associated with each corresponding reference point for each emission spot of individual light-emitting molecules.

In some examples, the zero-order image and first order image are generated simultaneously. In some examples, the zero-order image and first order image, localization information about individual emission spots of individual light-emitting molecules, and spectra information are and generated and captured simultaneously.

When data at multiple wavelengths are obtained however, it is possible to improve the contrast and detection sensitivity by spectral unmixing, e.g., by resolving the spectral signature of the absorption of the light-emitting molecules to be imaged over other non-specific spectral contributions, or from confounding signals from molecules with overlapping spectral signatures. In some examples, other types of light scattering or signals from non specific absorption (e.g. hemoglobin, or DNA), raman scattering may be removed using spectral unmixing.

Spectral unmixing methods based on differential or fitting algorithms use the known spectral information to process the image on a pixel-by-pixel basis. These methods try to find the source component (e.g., a distribution of a certain light-emitting molecule's emission) that best fits its known absorption spectrum in the least-squares sense.

There are numerous algorithmic methods for spectra unmixing known in the art. Generally, given the (n×m) multispectral measurement matrix M, where n is the number of image pixels and m is the number of measurements, as well as the (k×m) spectral matrix S with the absorption coefficients of the k components at the m measurement wavelengths, the data can be unmixed via Rₚᵢₙᵥ=MS, where S⁺ is the Moore-Penrose pseudoinverse of S and Rₚᵢₙᵥ is the reconstructed spatial distribution (e.g., image) of the chromophore of interest.

ii. Normalization, Spectral Regression for Classification of Molecule Emissions

Resolving individual spectral signatures in combination with emission spot localization of individual light-emitting molecules may allow for improved resolution. Individual spectral signatures can be resolved or distinguished for each localized emission spot for individual light-emitting molecules. In some examples, individual spectral signatures for 2 or more different molecules with the same absorption-emission band properties may be resolved. In some examples, individual spectral signatures for 2 or more different molecules with the same type of extrinsic label (e.g., both molecules may be labeled with rhodamine) may be resolved. In some cases, individual spectral signatures for 2 or more different molecules with 2 or more different types of extrinsic labels (e.g., molecules in a population may be labeled with many different extrinsic labels such as DAPI, rhodamine, GFP, RFP, YFP etc.) may be resolved.

### F. Image Processing

Various image-processing techniques may also be used to facilitate determination of the entities. For example, drift correction or noise filters may be used. Generally, in drift correction, a fixed point is identified (for instance, as a fiduciary marker, e.g., a fluorescent particle may be immobilized to a substrate), and movements of the fixed point (e.g., due to mechanical drift) are used to correct the determined positions of the switchable entities. In another example method for drift correction, the correlation function between images acquired in different imaging frames or activation frames can be calculated and used for drift correction. In some examples, the drift may be less than about 1000 nm/min, less than about 500 nm/min, less than about 300 nm/min, less than about 100 nm/min, less than about 50 nm/min, less than about 30 nm/min, less than about 20 nm/min, less than about 10 nm/min, or less than 5 nm/min. Such drift may be achieved, for example, in a microscope having a translation stage mounted for x-y positioning of the sample slide with respect to the microscope objective. The slide may be immobilized with respect to the translation stage using a suitable restraining mechanism, for example, spring loaded clips. In addition, a buffer layer may be mounted between the stage and the microscope slide. The buffer layer may further restrain drift of the slide with respect to the translation stage, for example, by preventing slippage of the slide in some fashion. The buffer layer, in one example, is a rubber or polymeric film, for instance, a silicone rubber film.

### III. Terminology

The terminology used therein is for the purpose of describing particular examples only and is not intended to be limiting of a device of this disclosure. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

Several aspects of a device of this disclosure are described above with reference to example applications for illustration. It should be understood that numerous specific details, relationships, and methods are set forth to provide a full understanding of a device. One having ordinary skill in the relevant art, however, will readily recognize that a device can be practiced without one or more of the specific details or with other methods. This disclosure is not limited by the illustrated ordering of acts or events, as some acts may occur in different orders and/or concurrently with other acts or events. Furthermore, not all illustrated acts or events are required to implement a methodology in accordance with this disclosure.

Ranges can be expressed herein as from “about” one particular value, and/or to “about” another particular value. When such a range is expressed, another example includes from the one particular value and/or to the other particular value. Similarly, when values are expressed as approximations, by use of the antecedent “about,” it will be understood that the particular value forms another example. It will be further understood that the endpoints of each of the ranges are significant both in relation to the other endpoint, and independently of the other endpoint. The term “about” as used herein refers to a range that is 15% plus or minus from a stated numerical value within the context of the particular usage. For example, about 10 would include a range from 8.5 to 11.5.

### IV. Examples

### Example 1

Certain examples determine stochastic fluorescence switching in nucleic acids under visible light illumination. By combining the principle of photon-localization microscopy, certain examples provide optical super-resolution imaging of native, unmodified DNA molecules; a technique referenced herein as DNA-PLM. Super-resolution imaging is then conducted from isolated, unstained chromosomes and nuclei, revealing nanoscopic features of chromatin without the need for exogenous labels. This paves the way for unperturbed, label-free nanoscale imaging of chromatin structure.

Results

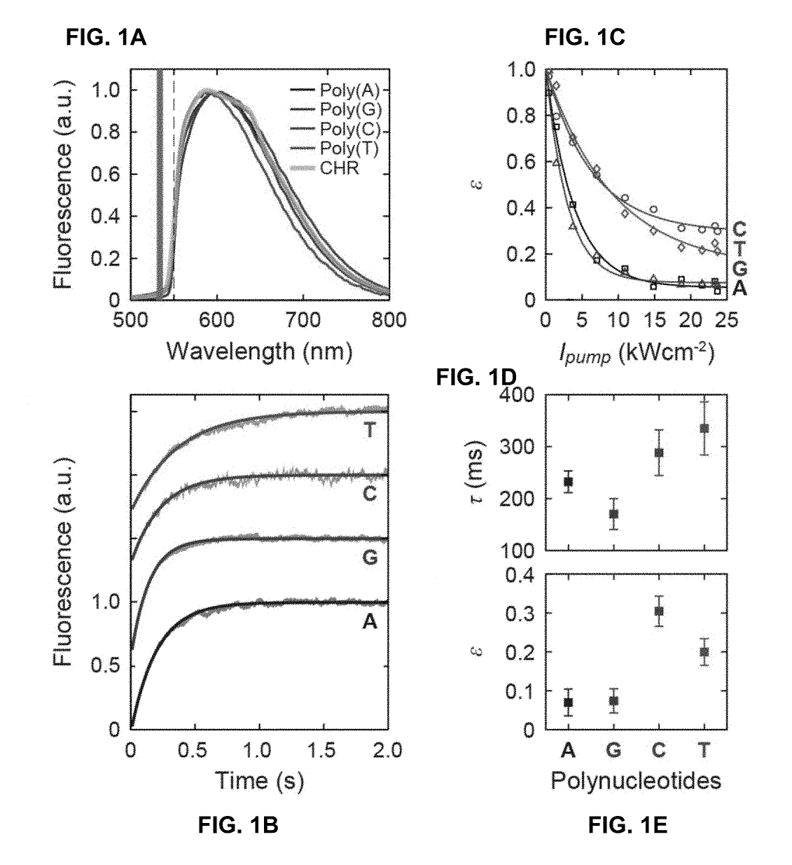

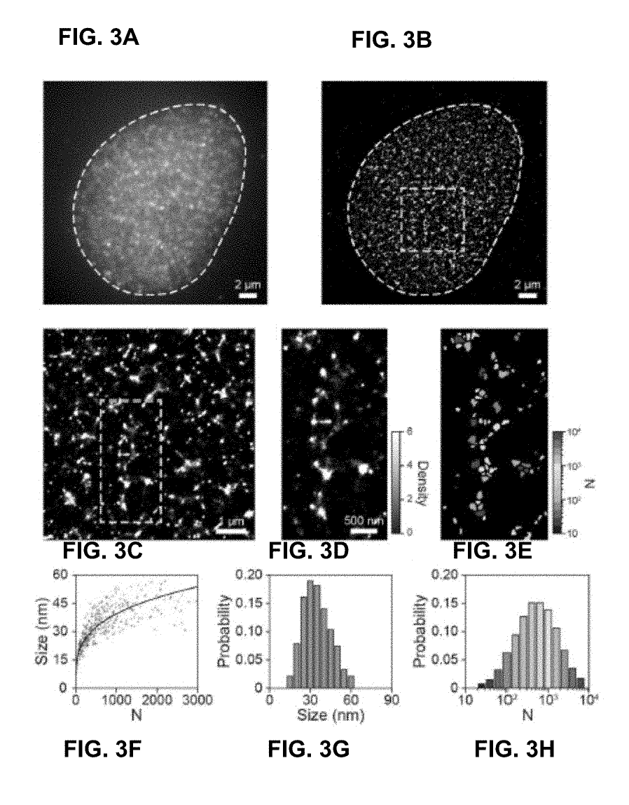

In our study, we first used short single-stranded polynucleotides (e.g., 20-bp poly-A, G, C, and T, IDT) as model systems to investigate the fluorescence excitation and photo-switching of DNA molecules. Although nucleic acids have significantly weaker absorption for visible versus UV light, they exhibit low, but detectable, absorption due to the electron delocalization effect, in part arising from the aromatic ring. As shown in the example of FIG. 1a, the fluorescence spectra of the four types of polynucleotides indicate peak emissions near 580 nm under 532-nm excitation. The measured spectra were consistent with the emission spectrum of chromosome samples studied in parallel (see sample preparation in Methods and corresponding discussion regarding FIG. 3a-h and 4a-e), which demonstrates that we are exclusively capturing fluorescence from DNA molecules.

Integration of intrinsic fluorescence with PLM requires the ability to achieve blinking single-molecule emission. Although fully mapping the electronic states in DNA molecules is a decades-old challenge, there is evidence indicating the existence of long-lived dark and triplet states with lifetimes as long as a few hundred milliseconds in nucleotides. These states can serve as primary candidates for photo-induced switching of nucleic acids by leveraging ground state depletion (GSD) with dark-state shelving and stochastic return. This phenomenon has previously been exploited for super-resolution microscopy with exogenous dyes. The corresponding photochemical process can be described by a system of three differential equations. Because the dark states have a lifetime, r, much longer than that of fluorescence, the majority of molecules are ‘shelved’ to their long-lived dark (e.g., triplet) states. Only a few molecules may return to their ground state at any given time, with the average rate of k=1/τ, where they can then be repeatedly excited to the fluorescent state. This creates the “on” and “off” periods, or blinking, yielding the required stochastic activities for precisely locating molecules with PLM.

The role of the long-lived dark state of polynucleotides was validated by a pump-probe method. As shown in FIG. 1b, the theory of GSD predicts that once GSD has been induced by a strong pump excitation (e.g., Iₚᵤₘₚ up to 24 kWcm⁻² for 100 ms) the fluorescence induced by a weaker probe beam (e.g., I_{probe}=0.3 kWcm⁻²) will follow the exponential time course of the repopulation of the ground state with recovery lifetime τ. Our results show that recovery lifetimes of polynucleotides are at hundred-millisecond level (FIG. 1b), which is consistent with the typical lifetime of the dark states for traditional fluorescent probes. Further validation of the GSD mechanism was achieved by varying Iₚᵤₘₚ and estimating the population of ground state, ε, as the ratio of fluorescence at the beginning of the recovery to the steady state. As expected, ε was inversely related to Iₚᵤₘₚ (FIG. 1c). FIGS. 1d and 1e further show comparisons of the recovery lifetime and population of ground state between polynucleotides using a beam fluence of 24 kWcm⁻², respectively. The recovery lifetimes of the four polynucleotides are within the same order of magnitude, which facilitate PLM with stochastic photon switching of all four types of nucleotides simultaneously in DNA molecules. Notably, different polynucleotides have distinct τ and ε. Among them, nucleotides containing purines (adenine and guanine) and pyrimidines (cytosine, thymine) have similar τ and ε, respectively, likely due to the similarity of their molecular structures.

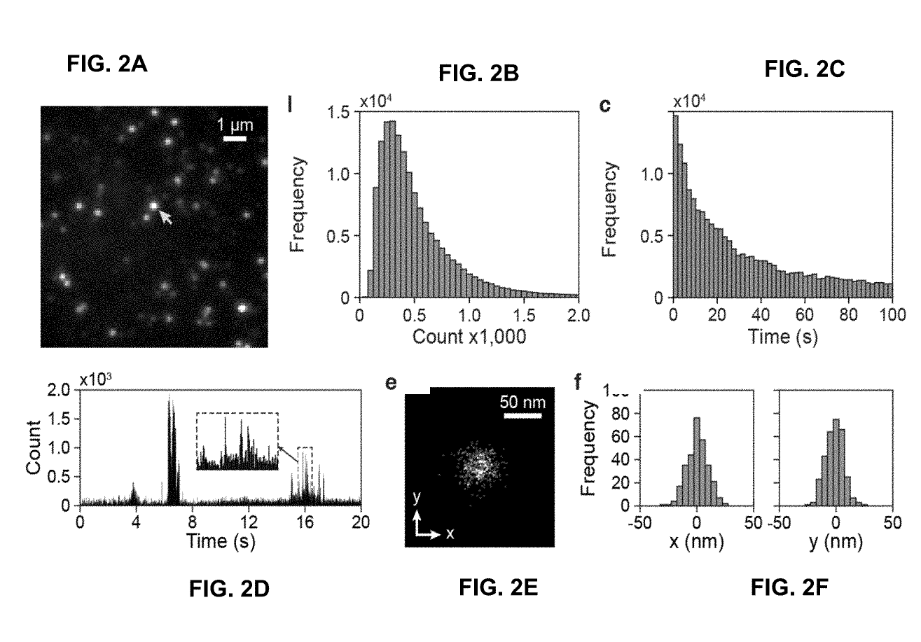

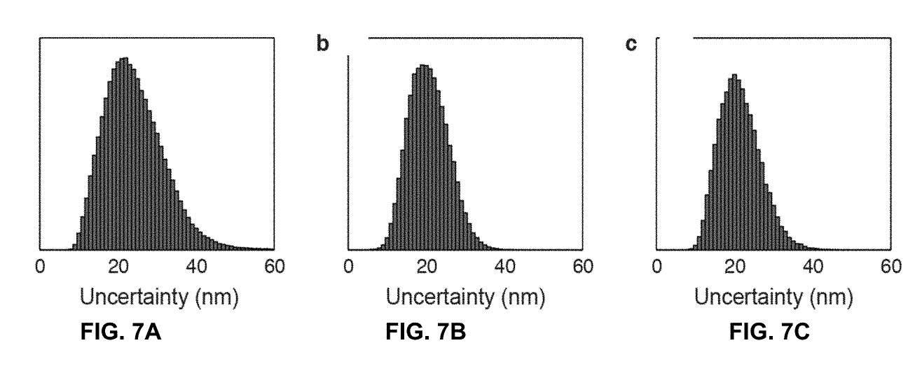

To demonstrate the imaging capability of DNA-PLM, certain examples perform single molecule imaging of 20-bp poly-G DNA (see detailed preparation in Methods). Poly-G DNA has a high dark state shelving probability and a relatively shorter recovery lifetime when compared to other investigated polynucleotides, making it ideal for demonstration. For imaging, Poly-G DNA samples are excited using a 532-nm laser with a fluence of 3 kWcm⁻², which is a lower level of excitation that balances the switching rate and the rate of photobleaching (which can turn the molecules irreversibly dark). Movies including 5,000 frames are acquired at exposure times of 10-ms per frame. As shown in FIG. 2a, the averaged wide-field fluorescence image shows only diffraction-limited features. Due to the stochastic nature of photon emission and dark state transition, the number of photons detected from a single molecule fluctuates. FIG. 2b shows a histogram of detected photon counts from each stochastic emission event, which shows a peak at ˜250 counts and an average at ˜550 counts. Based on the Nyquist criterion, DNA-PLM can theoretically achieve a spatial resolution of 22 nm due to the emission characteristics of polynucleotides (see FIGS. 7a-c). Next, we investigated the temporal characteristics of the stochastic fluorescence emission, as shown in FIG. 2c. The occurrence of stochastic emission events shows a temporal decay, which is characteristic of the exponential decay of photobleaching. Following the temporal decay stage, the stochastic emission reached an equilibrium state, with relatively stabilized stochastic emission frequency, lasting for more than 10 minutes before all molecules were photobleached.

Focusing on an individual molecule (as denoted by the arrow in FIG. 2a), we further studied the temporal properties of stochastic on-off switching from the time trace of the fluorescence signal (FIG. 2d). The average on-times were a few tens of milliseconds, whereas the off-times were significantly larger (ranging from several hundred milliseconds to 10 seconds). For the investigated molecule, the number of photons detected per fluorescence “on” event has an average of ˜500 counts but can burst up to 1,900 counts. This dramatic variation may be due to the natural complexity of the electrical structure in a DNA strand. After reconstruction of all stochastic fluorescence events, we generated a PLM image by plotting their centroids (FIG. 2e). The centroids approximately follow a Gaussian distribution with a full width at half maximum (FWHM) of 18±2 nm and 20±2 nm in horizontal and vertical direction, respectively (FIG. 2f), suggesting DNA-PLM achieves an imaging resolution of ˜20 nm. This is consistent with our previous estimation based on the Nyquist criterion. Notably, it has been reported that long-lived states in DNA base pairs joined by hydrogen bonds decay with essentially identical kinetics as those seen in single-strained polynucleotides. This suggests that double stranded DNA molecules with double helix should have similar photophysical properties as the single stranded polynucleotides being examined.

To demonstrate the label-free imaging of DNA topology in cells, nanoscale structure of interphase chromatin was imaged (see detailed preparation in Methods). FIG. 3a shows the wide-field fluorescence image of an isolated, unstained interphase HeLa cell nuclei. As indicated in FIG. 1a, the fluorescence spectrum of the sample is identical to that of polynucleotides, which demonstrated that the contrast is mostly from nucleic acids rather than proteins in the nuclei. FIG. 3b-c shows the corresponding DNA-PLM images at different scales. The macromolecular organization of nucleic acid structures is arranged in discrete nanoclusters in interphase nuclei, which is consistent with previous reports. The density image can be plotted by defining the density as the number of stochastic emission events per pixel (FIG. 3d). The density image was then converted into a binary image and segmented by grouping the emission events based on their proximity (FIG. 3e). The nanocluster size and the number of emission events in each nanocluster, N, was plotted in FIG. 3f. Furthermore, a quantitative analysis revealed the size distributions of nanoclusters (FIG. 3g) and the number of emission events per nanocluster (FIG. 3h), which can be useful in understanding the nanoscale organization of chromatin.

Investigation of chromatin organization and structure in interphase nuclei is important for gene function and activity. To date, super-resolution studies of chromatin with extrinsic labeling are accompanied by major drawbacks such as a limited ability to reveal the spatial organization of single or groups of nucleosomes and quantitatively estimating the nucleosome occupancy level of DNA. By imaging DNA molecules using intrinsic contrast, we provide a method to visualize the native structure of chromatin with nanoscale resolution. As the number of emission events per nanocluster reflects the relative length of DNA in each nanocluster, we found the size of the chromatin structure exhibits a power-law scaling behavior with respect to the DNA length with the scaling exponents of 0.28±0.03 (FIG. 3f). Consequently, even at these deeply subdiffractional length scales (20-60 nm) the topology of chromatin follows the same power-law structure as that observed at higher length scales. The value of the exponent close to ⅓ is consistent with the earlier proposed chromatin organization as a fractal globule: While a power-law, fractal globule relationship for chromatin organization has recently been observed in chromatin for clusters between 100-250 nm, certain examples demonstrate that this power-law topology extends down to tens of nanometers and thus the sub-kb scale. At these length scales, one possible explanation is that individual genes self-assemble into discrete clusters that maximize their surface area while minimizing their volume occupancy. In this case, transcription or replication of genes could only occur on the surface of the cluster, as the interior would be tightly packaged with nucleic acids. Alternatively, larger clusters could be more diffuse owing to the presence of active polymerases or replicases. A further exploration of this topology of chromatin can only be revealed by label-free techniques such as DNA-PLM, as extrinsic labels could have non-linear penetrance in such dense clusters. Additionally, a median cluster size of 30 nm is observed (FIG. 3g), which is consistent with other studies in fixed cells showing that chromatin assembles into the so-called 30 nm fiber in hypotonic conditions. While we have demonstrated that DNA-PLM is ideal for studies of nucleosome organization in isolated nuclei, as a non-invasive optical technique, DNA-PLM could potentially be suitable for nanoscopic imaging of chromatin in live cells. Through this extension, DNA-PLM would be the only technique capable of definitively answering lingering questions about the presence of the elusive 30 nm fiber in living eukaryotic cells.

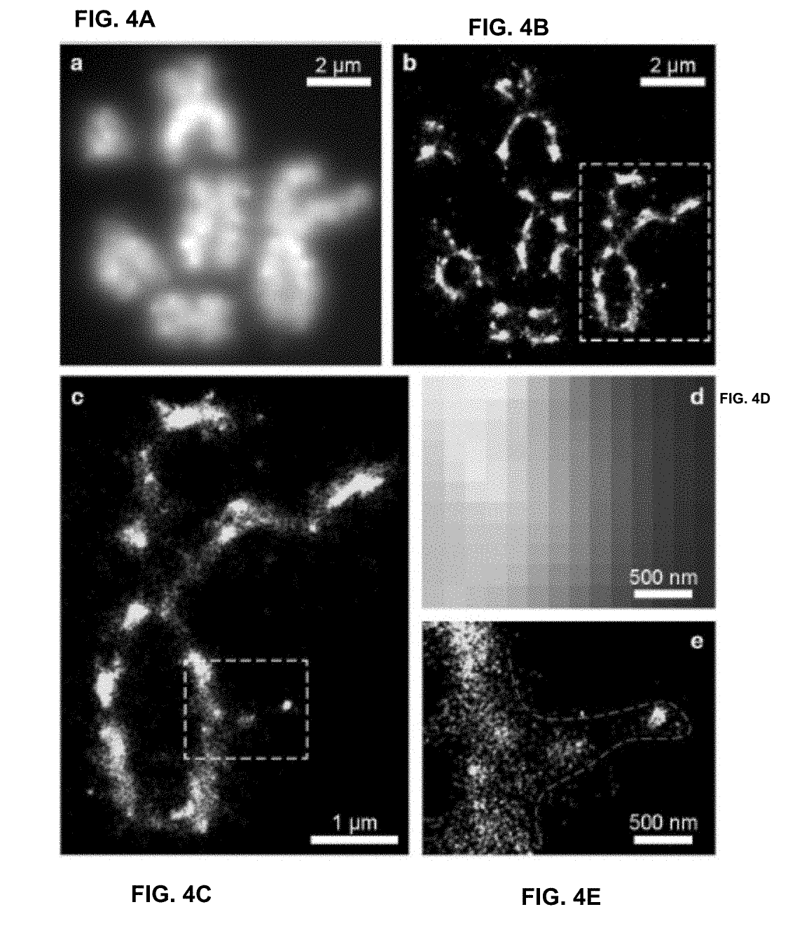

Next, we employed DNA-PLM to image the structure of isolated metaphase chromosomes. In particular, we focused on imaging auto-fluorescence of isolated X-chromosomes from HeLa cells, which can be readily observed under a wide-field microscope (FIG. 4a), however, with diffraction-limited resolution. Using DNA-PLM, we conducted super-resolution imaging of X-chromosomes (FIG. 4b). From higher zooms shown in FIG. 4c-e, we can clearly see variations in nucleotide density in the thick chromatids and the presence of a potential chromosomal fragile site; features which were not resolvable in the wide-field image. Chromosomal fragile sites are specific heritable points on metaphase chromosomes that tend to form a gap, constriction, or break when cells are exposed to a perturbation during DNA replication. Fragile sites frequently occur in the human genome and are classified as either common or rare based on their observed frequency. Observed common fragile sites are part of the normal chromosome architecture in all individuals and are of considerable interest in human diseases. In particular, common fragile sites are frequently transformed during tumorigenesis resulting in the loss of tumor suppressor genes or the formation of oncogenes. Likewise, rare fragile sites are seen in a small proportion of individuals, and are often associated with genetic disorders, such as Fragile-X syndrome. All fragile sites are susceptible to spontaneous breakage during replication, and as such their identification and study is important to understanding diseases, including cancer (FIG. 4e).

Discussions

Finally, we note that similar switching processes can be observed under other excitations with different wavelengths (e.g., tested by 488 nm, 445 nm and 405 nm laser illuminations). However, image quality usually suffered due to the more rapid photobleaching observed at shorter wavelengths, which limited the number of stochastic emission events acquired for image reconstruction. As these different wavelengths likely excite different singlet electronic states they can, however, be used to create new switching events or to return molecules from dark states. This is of particular use when emission events become rare due to photobleaching under prior excitation. Furthermore, specific imaging buffers, additives, or chemical methods used in chromatin fixation may vary the electronic state of DNA molecules and possibly be useful for suppressing the photobleaching or accelerating the switching of nucleotides. Follow-up studies are merited to fully understand the photophysics of DNA molecules under various conditions.

In summary, in certain examples, we have investigated the photo-switching process of native, unmodified DNA molecules and demonstrated the super-resolution imaging capability of DNA-PLM. Using DNA-PLM we can achieve sub-20-nm resolution with unmodified DNA molecules. This is particularly suitable for imaging chromatin structures and may allow insight into native structures of DNA organization in cells. Understanding and controlling the mechanisms for photo-induced dark state formation in DNA molecules is important to develop better switching, to optimize the imaging parameters, and to apply DNA-PLM to study chromatin organization in live cells. With further development, combined with temporal and spectral characterization, DNA-PLM can feasibly identify highly specific molecular “fingerprints”, leading to in-situ label-free sequencing of the genome. Additionally, topological and chemical alterations in highly condensed DNA strains can result in various additional photophysical interactions, as has been studied in polymer molecules, including energy transfer, ground- or excited-state aggregate formation, and charge transfer. These photophysical processes can significantly modify the molecular optical properties, allowing us to further capture functional information about the chromatin nanoarchitecture.

Methods

Fluorescence Characterization

For studying the fluorescence characteristic of polynucleotides, we built an integrated optical imaging and spectroscopy system based on an inverted microscope. For example, a 532 nm diode-pumped solid-state laser with 300-mW maximum output was passed through the microscope body (e.g., Nikon, Eclipse Ti-U) and was focused by an objective lens (e.g., Nikon, TIRF 100×, 1.49NA). The intensity and beam size of the illumination beam fluence were controlled by a linear polarizer and a dual lens assembly. For spectral characterization, the signal was routed to a spectrometer (e.g., Princeton, SP2150i) with a 150 lines/mm diffraction grating and an EMCCD (e.g., Princeton Instruments, ProEM512B Excelon), giving a maximum 0.6-nm spectral resolution. A long-pass filter (e.g., BLP01-532R-25, Semrock) was used to reject the reflected laser beam. The primary fluorescence image was collected through a 550-nm long-pass filter before video acquisition by an EMCCD (e.g., Andor, iXon 897 Ultra) at a frame rate of 100 Hz. We determined the fraction of residual singlet state molecules using a pump-probe mode with a constant probe (e.g., 0.3 kWcm⁻²) and pump pulses of varying intensity (e.g., 100 ms, 1-25 kWcm⁻²) for shelving the molecules into dark states. The fluorescence recovery was monitored for calculating the recovery lifetime by applying an exponential fitting.

Preparation of Polynucleotides Hydrogel and Single Molecule Samples

In certain examples, 10 μL polynucleotides (5′-AAA AAA AAA AAA AAA AAA AA-3′ (SEQ ID NO. 6), 5′-GGG GGG GGG GGG GGG GGG GG-3′ (SEQ ID NO. 7), 5′-CCC CCC CCC CCC CCC CCC CC-3′ (SEQ ID NO. 8), 5′-TTT TTT TTT TTT TTT TTT TT-3′ (SEQ ID NO. 9)) solution (e.g., 100 μM, IDT) were dropped on a coverslip (e.g., #1.5, Tedpella) surface and dried at 20° C. overnight to form hydrogel thin films. Single molecule polynucleotide samples were prepared by diluting the polynucleotide solution 10,000 times with nuclease-free water (IDT) and fixing with poly-L-lysine (Sigma-Aldrich) on the coverslip surface. After incubating for 10 minutes, samples were then washed 3 times by PBS buffer and then sealed with PBS buffer, for example.

Chromosome Preparation from Cultured Cells

Chromosome and nuclei isolation was performed as described previously with minor modifications. In brief, samples were isolated from HeLa cells (e.g., passage 10-15) grown into log phase (e.g., >80% confluence) and treated for 90 minutes with 2.5 mg/ml of colchicine to arrest cells during M-phase. Following colchicine treatment, cells were washed with 1×PBS (e.g., pH 7.4), trypsinized, and pellet was isolated by centrifugation at 200×g for 10 minutes. Following isolation, pellet was resuspended in hypotonic KCl solution (e.g., 0.075M) for 10 minutes at 37° C. Finally, pellets were fixed in Carnoy's fixative for 10 minutes and washed 3 times prior to deposition on coverslips. Prior imaging, 5 μL nuclease-free water (e.g., IDT) was dropped at the center of a freshly cleaned glass slide, and the sample on the coverslip was mounted on the glass slide and sealed with dental cement.

DNA-PLM Imaging Process

Chromosome samples were placed on the microscope stage and imaged using a high-NA TIRF objective. Before acquiring DNA-PLM images, we used relatively weak 532-nm light (e.g., ˜0.3 kWcm⁻²) to illuminate the sample and recorded the conventional fluorescence image. We then used a 532-nm laser with constant beam fluence of 3 kWcm⁻² to switch a substantial fraction of DNA molecules to their “off” states. After exposing under laser beam illumination for at least 5 minutes, we started to record images in the stabilized switching stage using the EMCCD camera (e.g., iXon Ultra 897, Andor). The integration time and the frame rate of image acquisition were carefully selected to provide optimal signal-to-noise ratio of the acquired image. Unless specifically noted, 10,000 frames were recorded for PLM reconstruction.

### Example 2

Nucleic acids have significantly weaker absorption for visible versus UV light. However, they exhibit low, but detectable, absorption in the visible range due to the electron delocalization arising from the aromatic rings. This is a fundamental property critical to their molecular function and stability. Visible light absorption by nucleic acids have been measured and the data are readily available. The molar extinction of nucleotides in the visible is E˜50 cm⁻¹ M⁻¹, which is 260 times lower than their UV absorption and >1,000 times lower than the peak absorption of strong extrinsic fluorophores, such as rhodamine. We recorded fluorescence from mononucleotides, nucleic acid bases, and short single-stranded polynucleotides (e.g., 20-bp poly-A, G, C, and T, IDT). This radiative process was consistent with endogenous fluorescence; the emission excited at 532 nm by a pulsed laser had a lifetime τ_{fl}˜2 ns, which is typical of fluorescence lifetimes of high-Q fluorophores.

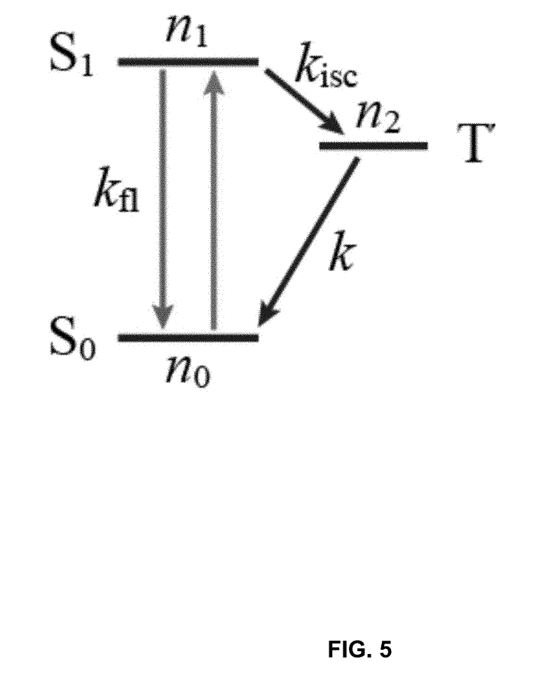

Integration of endogenous fluorescence with PLM requires the ability to detect blinking single-molecule emission. We have accomplished this feat by leveraging ground state depletion (GSD) with dark-state shelving and stochastic return. The phenomenon has previously been exploited for super-resolution microscopy with exogenous dyes. When excited by light with intensity Iₑₓ, a molecule transitions from its ground state (S₀) to an excited state (S₁) with the average rate kₑₓ=Iₑₓσ/hv, where a is the absorption cross section and v is the frequency of the transition (FIG. 5). From this state the molecule can relax non-radiatively; emitting a fluorescence photon with probability Q or transition to a dark (e.g., triplet) state (T) via intersystem crossing (ISC) with probability Φ<<1. Because the dark states have a lifetime T much longer than that of fluorescence (e.g., τ>100 ms>>τ_{fl}˜ns), with each excitation molecules increasingly shelve in a long-lived dark state. While in this long-lived dark state, the molecule no longer fluoresces. However, it may return to the ground state with the average rate k=1/τ after which it is again available for excitation. This creates the “on” and “off” periods, or blinking. The process can be described by a system of three differential equations:

$$\{ \begin{matrix}\frac{{dn}_{0}}{dt} = - {k}_{ex} {n}_{0} + {k}_{f l} {n}_{1} + {kn}_{2} \\ \frac{{dn}_{1}}{dt} = + {k}_{ex} {n}_{0} - {k}_{f l} {n}_{1} - {k}_{isc} {n}_{1} \\ \frac{{dn}_{2}}{dt} = + {k}_{isc} {n}_{1} - {kn}_{2}\end{matrix} ,$$

where n_{0,1,2} are the population probabilities of the molecule and Σᵢnᵢ=1. k_{fl}=1/τ_{fl} and k_{isc}=1/τc_{isc}, where τ_{fl} and τ_{isc} is the fluorescence lifetime and the intersystem crossing lifetime, respectively.

At steady-state, the fraction of the fluorophores that are in the ground state (and can generate fluorescence) was reduced by increasing Iₑₓ as ε≈1/(1+kₑₓτΦ). If Iₑₓ>>hv/(σΦ), the majority of molecules are ‘shelved’ in the dark (e.g., triplet) state. With few molecules available for excitation, single-molecule blinking fluorescence may be observed. The average “on” time is τ_{B}=1/(kₑₓΦ), the fluorescence photon arrival rate during the “on” period is σ=Qkₑₓ, and the photon count per each blinking is N_{B}≈Q/Φ.

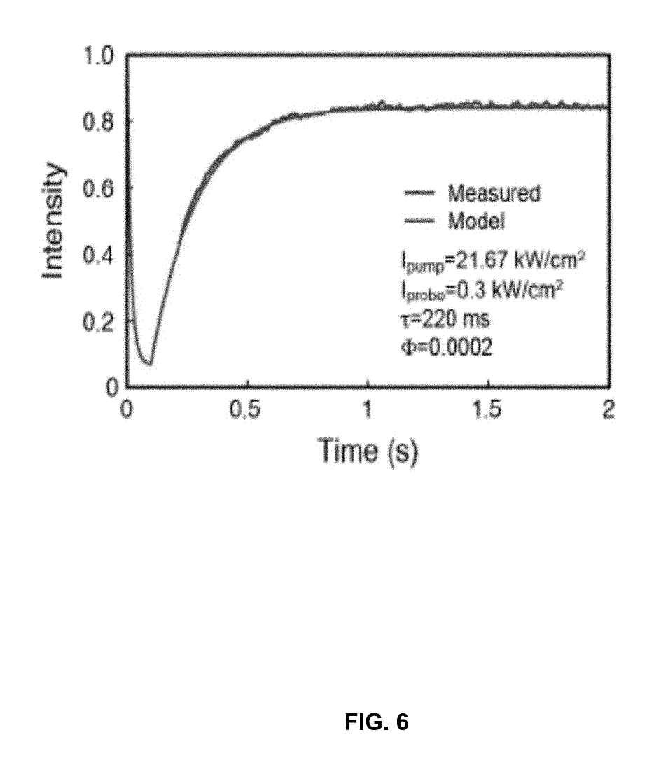

Rigorous study of the mechanism of the endogenous fluorescence blinking of polynucleotides (e.g., 20-bp poly-A, G, C, and T) supports the GSD mechanism. First, we validated the role of the long-lived dark state in the observed stochastic emission of nucleic acids. The theory of GSD predicts that once GSD has been induced by a strong pump excitation (e.g., Iₚᵤₘₚ up to 24 kWcm⁻² for 100 ms), the fluorescence induced by a weaker probe beam (e.g., I_{probe}=0.3 kWcm⁻²) will follow the exponential time course of the repopulation of the ground state with recovery timescale τ. Our data shows that the recovery time for polynucleotides samples is τ˜150-400 ms, which is typical for the lifetime of the triplet states. The experimental recovery data fits accurately with the GSD model (FIG. 6), with σ taken from the published data (e.g., ˜1,000 M⁻¹ cm⁻¹ for the 20-bp DNA) and Φ≈0.0002 (found as a fitting parameter). The value of Φ is also characteristic of the intersystem crossing into a triplet state. Finally, addition of the triplet-specific quencher, β-mercaptoethanol, reduced τ by 36%, thus confirming the shelving of excited electrons in the dark, and most likely, triplet state.

Further validation of the GSD mechanism was achieved by varying Iₚᵤₘₚ intensity and estimating ε as the ratio of fluorescence at the beginning of the recovery, read by I_{probe}=0.3 kWcm⁻², to the steady state probe fluorescence. As expected, ε≈1/(1+IₚᵤₘₚτΦ) was inversely related to Iₚᵤₘₚ intensity.

Remarkably, the photochemical characteristics of nucleic acids under visible light illumination make them ideal candidates for use as GSD-blinking fluorophores in biological systems: (1) They exhibit a long shelving lifetime, τ, which is ideal for efficient depletion. (2) Although nucleic acids have weak fluorescence due to a low absorption in bulk, the photon number of individual emission events is comparable to most exogenous dyes used in PLM. (3) Finally, detection at lower excitation intensity (˜5 kWcm⁻²) is highly advantageous compared to the cell-damaging high light intensities typically used in some other super-resolution approaches (e.g. up to 10⁵ kWcm⁻² in STED). However, if r was substantially longer, it would slow down image acquisition.

### Example 3

In Raman scattering and fluorescence excitation and emission, incident photons interact with the intrinsic electronic or vibrational states of the sample and subsequently emit frequency-shifted photons due to the underlying energy exchange. Analyzing the spectroscopic signatures obtained from inelastic light scattering measurements is a widely used method for revealing the electronic and structural properties for natural and engineered materials in subjects ranging from biology to materials science. Additionally, a variety of spectroscopic imaging techniques have been developed to probe the heterogeneous environment within samples, yet their spatial resolutions have been limited to about half of the wavelength due to light diffraction. Although near-field scanning optical microscopy (NSOM) offers nanometer-scale spatial resolution by using a sharp stylus for scanning at the close vicinity of the sample surface, it is unable to image sub-surface features because rapidly decaying evanescent fields are accessible only within the optical near-field. Therefore, further development of far-field spectroscopic nanoscopy remains highly desirable.

Recent advancements in super-resolution fluorescence microscopy have extended the ultimate resolving power of far-field optical microscopy significantly beyond the diffraction limit. A wide range of imaging modalities, including structured illumination microscopy (SIM), stimulated emission depletion microscopy (STED), and photon localization microscopy (PLM), have been successfully developed. In particular, PLM, which includes photoactivated localization microscopy (PALM) and stochastic optical reconstruction microscopy (STORM), relies on the stochastic radiation of individual fluorescent molecules to determine the probabilistic locations from their localized point spread functions (PSFs) while providing deep sub-diffraction-limited spatial resolution. Notably, PLM does not alter the emission spectrum of the stochastic radiation, making it promising for the development of a spectroscopic nanoscope. Previously, analyzing the spectroscopic features of the stochastic radiation of single molecules has been demonstrated by recording multiple images from discrete wavelength bands. However, due to the limited imaging sensor area of a single CCD camera, only several wavelength bands can be recorded simultaneously. The resulting poor spectral resolution makes this method unable to resolve fine spectral details and distinguish spectra with overlapping emission bands. Further improvement of spectral resolution is limited to the overall size of the imaging sensor array, and it can be rather complicated and expensive if multiple cameras are employed.

Here we report spectroscopic photon localization microscopy (SPLM), a newly developed far-field spectroscopic imaging technique, which is capable of simultaneously capturing multiple molecular contrasts from individual molecules at nanoscopic scale. Other approach with much lower spectral resolution was recently reported for multicolor super-resolution imaging; however, SPLM's novel optical design permits sub-nanometer fluorescence spectral analysis of individual molecules at sub-10 nanometer spatial resolution, for example. Using a slit-less monochromator, both the zero-order and the first-order diffractions from a grating were recorded simultaneously to reveal the spatial distribution and the associated emission spectra of individual stochastic radiation events, respectively. Whereas conventional PLM analyzes only the centroid of each stochastic radiation event, SPLM further captures and correlates the associated emission spectrum with the location of centroids. By employing spectral unmixing and regression, we successfully demonstrated nanoscopic spectroscopic imaging of multi-labeled cells with spatial resolution of 10 nm and spectral resolution of up to 0.63 nm, for example. Our approach not only enhances existing super-resolution imaging by capturing the molecule-specific spectroscopic signatures, it will potentially provide a universal platform for unraveling heterogeneous nanoscale environments in complex systems at the single-molecule level.

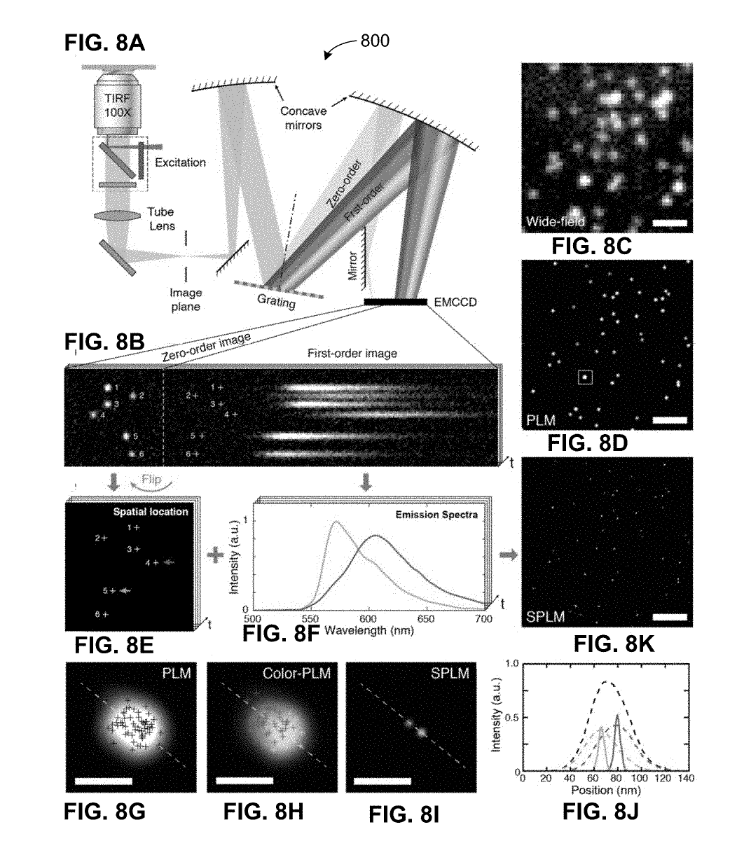

The working principle of SPLM is schematically illustrated in FIGS. 8a-e. As shown in FIG. 8a, a continuous-wave laser illumination was used to excite the fluorescent molecules into long-lived dark states and subsequently recover them by stochastic photo-switching. The resulting fluorescence image was coupled into a Czerny-Turner type monochromator (e.g., SP2150, Princeton Instruments) featuring a blazed dispersive grating (e.g., 150 grooves/mm). The collected fluorescent emission was divided at an approximate 1:3 ratio between the zeroth and the first diffraction orders. To simultaneously acquire the zero-order and first-order images using a high-sensitivity EMCCD camera (e.g., proEM, Princeton Instruments), a mirror was placed in the monochromator to adjust the position of the zero-order image. This helps establish temporal and spatial correlations between zero-order and first-order images in dealing with the sparsely distributed emissions which are stochastic by nature.

We first investigated a dual-stained sample with mixed actin monomers (Cytoskeleton) labeled with Alexa Fluor 532 (Life Technologies) and Alexa Fluor 568 (Life Technologies). Our sample was excited using a 532 nm laser with a beam fluence of 2 kWcm⁻². A movie containing a time sequence of one thousand image frames was recorded with a 20 ms exposure time for each frame. Each image frame contained simultaneously captured zero-order and first-order images. A captured frame shown in FIG. 8b is used as a representative example to illustrate the working principle of SPLM. The zero-order image does not impose additional dispersive characteristics of the grating, and thus it can be used to localize the positions of individual stochastic radiation events. The remaining photons are allocated to the first diffraction order to form a spatially dispersed image that reveals the fluorescence spectra of individual fluorescence dye molecules. For illustration, six stochastic radiation events and their corresponding emission spectra are numbered in FIG. 8b. Each stochastic radiation event originating from a single dye molecule remains spatially confined as a sub-diffraction-limited point source. Thus, it is possible to eliminate the need for a monochromator entrance slit without compromising the spectral resolution. This is particularly beneficial, as it allows for simultaneous acquisition of stochastic radiation events over a wide field-of-view and the associated fluorescent spectra.

Thus, FIG. 8a illustrates SPLM according to certain embodiments or examples. As shown in the schematic of the SPLM system 800, upon laser excitation, the fluorescent image is collected by a high-NA objective lens and subsequently coupled into a Czerny-Turner monochromator by a match tube lens. As shown in the example of FIG. 8b, both the zero-order and the first-order diffractions from the grating can be recorded simultaneously using the same EMCCD camera. As shown in FIG. 8c, wide-field optical image of the sample can include actin monomers labeled by Alexa Fluor 532 and Alexa Fluor 568 with diffraction-limited resolution. FIG. 8d shows that the conventional PLM method offers sub-diffraction-limited imaging resolution, but is unable to capture the spectroscopic signature of the individual emitter. Bars: 1 μm. In the example of FIG. 8e, the localization algorithm was used to determine the spatial locations of each blinking, illustrated by numbered crosses. These locations can be further used as the inherent reference points for spectral calibration of the emission spectra in the first-order image, shown as denoted crosses in FIG. 8b. FIG. 8f shows representative spectra from two individual blinking events (highlighted by the colored arrows in FIG. 8e). FIG. 8g shows an example magnified view of the square region in the PLM image (FIG. 8d). FIG. 8h shows a corresponding color-coded image by separating the spectra of individual stochastic localizations according to the emission characterization of the two dyes. The spectral regression of nearby localizations indicates the cluster consisted of two single-dye molecules. As shown in the example of FIG. 8i, by averaging the nearby localizations, the SPLM image with spectral regression shows the localization precision of two molecules. Bars: 50 nm. In the example of FIG. 8j, line profiles were used to compare the localization precision of PLM (black dashed line), color-PLM (colored dashed lines) and SPLM (colored solid lines). In the example of FIG. 8k, the super-resolution spectroscopic image was obtained by combining the spatial and spectroscopic information from all localizations. Bar: 1 μm.

FIG. 8c shows a wide-field fluorescence image of the sample with diffraction-limited resolution. While PLM is capable of providing much improved spatial resolution by recording and analyzing a movie containing sparsely distributed emissions (FIG. 8d), the lack of spectroscopic information makes PLM unable to distinguish different fluorescence dyes based on their spectroscopic signatures. In contrast, SPLM simultaneously records both spatial position and spectroscopic information. As shown in FIG. 8e, the zero-order images are analyzed by using the standard localization algorithm (e.g., QuickPALM, ImageJ plug-in) to determine the locations of individual blinking events, which is identical to the processing used in STORM and PALM. The mirror flipped the zero-order images horizontally, but this transformation was easily reversed during image processing. The centroid position serves two roles: (1) to determine the location of each activated fluorescent molecule (shown as numbered crosses in FIG. 8e) and (2) to establish a reference point to the corresponding emission spectrum from the measured first-order image (shown as numbered crosses in FIG. 8b).

To obtain optimal spatial and spectral resolutions, background signals, such as auto-fluorescence, Raman, or Rayleigh scattering from the sample, must be carefully removed. In this work, the background signals were removed by subtracting the average of the adjacent image frames without the presence of stochastic emission. The recorded spectrum from first-order image was further normalized by the wavelength-dependent characteristic of optical components and EMCCD. The dispersion of the imaging system was calibrated prior to image acquisition. Taking into consideration the focal length of the monochromator, the dispersive characteristic of the grating, and the pixel size of the EMCCD camera, we achieved a spectral resolution of 0.63 nm/pixel from the recorded first-order image. FIG. 8f shows the spectra from two individual blinking events, which match reasonably well with the emission spectra of fluorescent dyes being used: Alexa Fluor 532 and Alexa Fluor 568, for example. In FIG. 8e, the arrows of matching color highlight the corresponding spatial locations for the spectra. Given the sparse nature of the stochastic emissions, the measured spectra from neighboring fluorescence molecules are unlikely to overlap in space. In the rare event in which overlap occurs, the spectra of neighboring fluorescence molecules can be separated with a customized spectral unmixing algorithm.

The demonstrated capability of SPLM to distinguish minor differences in fluorescent spectra offers unique advantages compared with conventional PLM. FIG. 8g shows a magnified view of the highlighted region in the conventional PLM image (FIG. 8d). Every localization event is convolved with a Gaussian kernel, where the full-width at half-maximum (FWHM) is determined by the localization precision. It appears that the multiple stochastic emissions are clustered in close proximity. However, by examining the emission spectra from individual stochastic emissions using SPLM, we discovered that the emissions actually originated from two different types of fluorescent dye molecules. By color-coding each localization event with its spectral signature, we can determine that the centroids of two fluorescent molecules are spatially separated (FIG. 8h). Given the level of the dilution of the dye molecules, it is reasonable to expect that the observed clusterings of stochastic emissions are originated from single dye molecules. Using this knowledge, a spectral regression algorithm can be applied to identify the emissions from the same dye molecules and subsequently to accumulate all the photons for the localization analysis. With this technique, localization precision can be improved to sub-10 nm. The two fluorescent molecules with 15 nm center-to-center spacing can be clearly distinguished (FIGS. 8i and 8j).

Notably, since only one-fourth of the total photons emitted was allocated to the zero-order image, it resulted in a two-fold reduction in the spatial resolution according to the localization precision. Experimentally, we have observed ˜40 nm spatial resolution in this study, which suggests a theoretical resolution limit of ˜20 nm if all emitted photons were allocated to the zero-order image. By applying the spectral regression algorithm, photons from the multiple stochastic emissions from the same fluorescent dye molecule can be combined to improve the imaging resolution. As an example shown in FIG. 8d, the recording of 23,924 localizations can be classified to 1,582 clusters, indicating an average repeated occurrence of 15.1 under the given imaging buffer and laser excitation conditions. The resolution analysis based on localization precision shows the dramatically improved spatial resolution from 39.0 nm to 9.8 nm by applying spectral regression algorithm, which is more than a two-fold improvement over the standard PLM method. Nevertheless, the ultimate spatial resolution depends on the total number of photons emitted by individual dye molecules, which is eventually determined by the irreversible photo-bleaching threshold. Finally, super-resolution spectroscopic imaging can be accomplished, as illustrated in FIG. 8k.

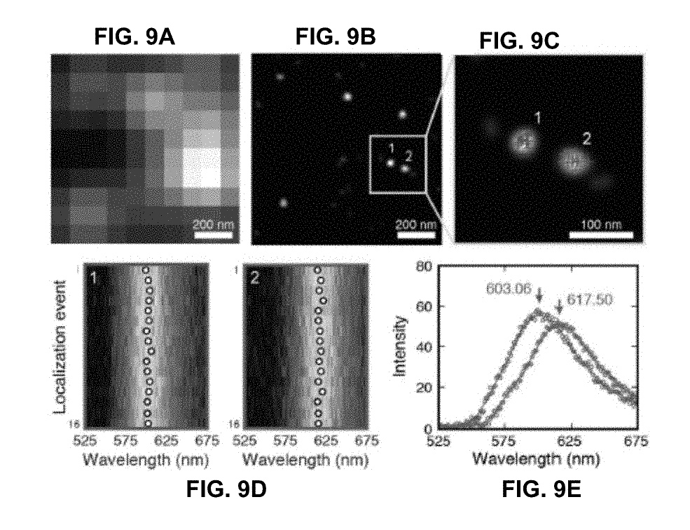

Even for the same type of molecules, individual molecules can be differentiated by exploiting their heterogeneous fluorescence. To verify this, we imaged actin monomers labeled solely with Alexa Fluor 568. FIG. 9a shows a wide-field fluorescence image, and FIG. 9b shows the corresponding conventional PLM image. FIG. 9c shows a magnified view of two clusters from the yellow box in FIG. 9b. The dye molecules can be repetitively activated, and their stochastic emissions can be recorded in multiple frames. The evolutions of their fluorescence spectra are shown in FIG. 9d. Each individual cluster was found to exhibit repeatable emission spectra with a small variation in the peak position of 2.25±0.45 nm. In contrast, different clusters exhibited clearly distinguishable spectra. As shown in FIG. 9e, the peak positions of the averaged fluorescence spectra for cluster #1 and #2 were 603.1 nm (e.g., standard deviation (SD)=1.8 nm) and 617.5 nm (e.g., SD=2.1 nm), respectively, with a corresponding wavelength shift of 14.4 nm. The observed heterogeneous fluorescent spectra from molecules of the same type appear to be caused by molecular conformational variations and environmental heterogeneity. These findings led us to believe that the multiple stochastic radiation events within a localized cluster were originating from the same dye molecules, which can be used to identify the origin of stochastic emission, judging by proximity in space and similarity in the emission spectra.

Thus, FIG. 9a illustrates an example wide-field optical image and FIG. 9b shows an example PLM image of actin monomers labeled by Alexa Fluor 568. The PLM image was reconstructed from localization coordinates using localization precision as the FWHM of a Gaussian kernel. In the example of FIG. 9c, two nearby clusters are highlighted and localization coordinates are marked by crosses. The example of FIG. 9d shows emission spectra denoted by colored circles in FIG. 9d. FIG. 9e illustrates the corresponding averaged spectra of these two clusters showing distinct emission peaks.

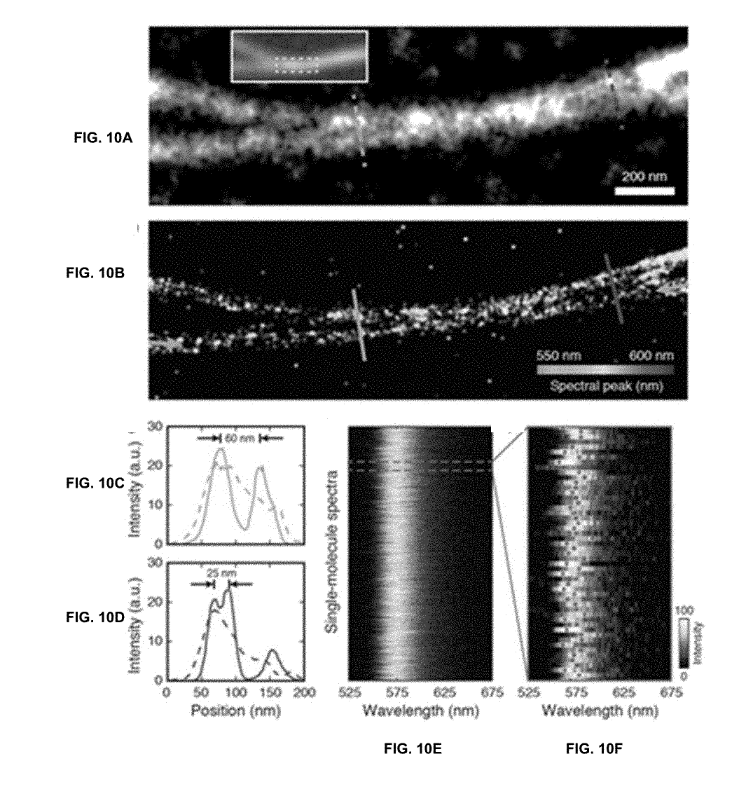

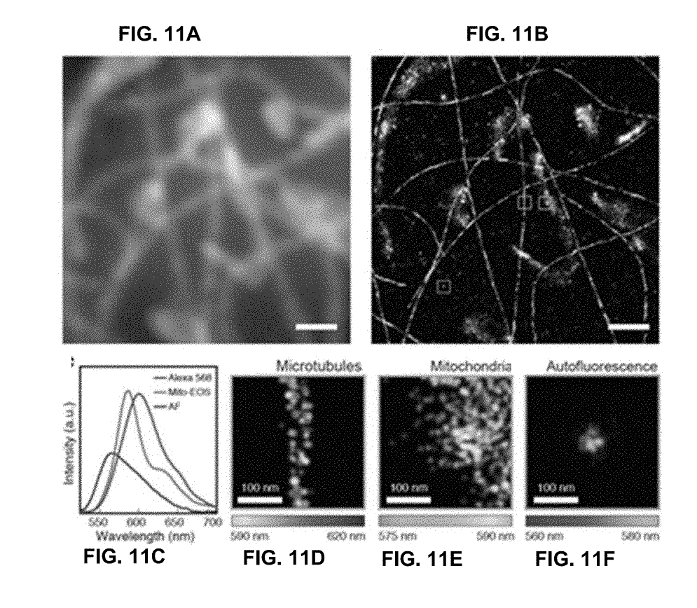

We further validated the improved SPLM imaging resolution using the Rhodamine-labeled microtubule samples. FIG. 10a shows a conventional PLM image of two closely spaced microtubules. After applying spectral regression, we rendered a SPLM image, as shown in FIG. 10b, with the color representing the peak wavelength of each molecule. As shown by the line profiles in FIG. 10c, the two microtubules, which were difficult to distinguish from each other in the PLM image (dashed line), can be clearly resolved in the SPLM image (solid line). Features as small as 25 nm can be resolved from single microtubules (FIG. 10d). FIG. 10e shows the spectra of all localization events in one of the microtubules, indicated by arrows in FIG. 10b. As illustrated in the magnified view (FIG. 10f), dye molecules of the same type have variations in spectral emissions due to the underlying fluorescent heterogeneity.

Thus, FIGS. 10a-f show example imaging of ex vivo microtubules using SPLM. FIG. 10a shows a conventional PLM image of two closely spaced microtubules of the square region in the wide-field fluorescence image, as shown in the inset. FIG. 10b SPLM image with spectral regression. FIGS. 10c and 10d are the line profiles from positions highlighted by the dashed- and solid-lines in FIGS. 10a and 10b, respectively. FIG. 10e shows emission spectra along a single microtubule, highlighted by the arrows in FIG. 10b. FIG. 10f shows a magnified view of the spectral variation. The circles indicate the peak positions of each spectrum.

Another crucial advantage of SPLM is that it may enable multi-label super-resolution imaging from a single round of acquisition. We demonstrated this advantage of SPLM by imaging dual-stained COS-7 cells. We used Alexa Fluor 568 and Mito-EOS 4b to stain the microtubules and mitochondria of a cell culture, respectively. FIGS. 11a and 11b show a wide-field fluorescence image and a reconstructed SPLM image, respectively, of a dual-stained COS-7 cell. In the SPLM image, different colors represent the spectral peak wavelengths of different molecule. As shown in FIG. 11c, Alexa Fluor 568 has a single emission peak at 600 nm, whereas Mito-EOS 4b has a main emission peak at 580 nm and a weaker peak at 630 nm. Although the stains have similar fluorescent colors, SPLM can easily identify them due to their distinct emission spectra, which was previously challenging in reported multicolor super-resolution approaches due to limited spectral resolution. As shown in FIG. 11d-f, magnified views of regions highlighted by the colored squares show details from different contrasts.

Thus, the example of FIG. 11a-f illustrates multi-labeled SPLM imaging. The example of FIG. 11a shows a wide-field fluorescence image of a dual-stained COS-7 cell. FIG. 11b shows a corresponding SPLM image. Bar: 1 μm. FIG. 11c shows fluorescence emission spectra of Alexa Fluor 568, Mito-EOS 4b, and the distinct emission spectrum from the background. The three colors represent the spectral peak wavelengths of different molecules. Magnified views of the regions inside the colored squares show (FIG. 11d) a single microtubule, (FIG. 11e) the edge of mitochondria, and (FIG. 11f) a spot with autofluorescence emission.

Finally, SPLM can be used to identify artifacts from the background. As shown in FIG. 11b, we determined scattered localization events that feature distinct emission spectra other than that of the exogenous dye molecules. These events are most likely from endogenous autofluorescence or from unknown sources of fluorescence induced by the use of fixatives or DNA transfection reagents. This phenomena is overlooked by conventional PLM and may be blamed illegitimately on unspecific antibody binding; however, our SPLM method provides the capabilities to reveal the potential imaging artifacts and develop a deeper understanding of their origins.

Use of the emission spectrum to discern the labels of fluorescent dye molecules constitutes a methodological advancement over the sequential recording used in previous multicolor experiments. Simultaneously characterizing multiple dye molecules with their spectroscopic information largely extends the combination of discernable markers and improves imaging speed in multi-stained samples. It also provides the capability to discern imaging artifacts originated from autofluorescence through their distinct emission spectra. Additionally, the demonstrated ability to distinguish the minor difference in the fluorescent spectra allows the identification of individual molecules, even among the same type of molecules. By using the spectral regression algorithm in this way, we can achieve higher spatial resolution through better use of the photons emitted by individual emitters. Moreover, image acquisition speed can be further improved by balancing the image SNR and the spectral resolution. In practical applications, high spectral resolution may not be required for identifying the vast majority of fluorescent molecules. Using the lower groove density of the grating or the shorter monochromator focal length can improve the SNR, since the available photons from each single-molecule emission occupies fewer pixels in the spectral image. This also reduces spectral overlapping and, thus, increases throughput, namely, the number of spectra that can be distinguished in one frame. Overall, the image recording can be accelerated sequentially to achieve the desired temporal resolution for particular applications.

Despite the success of electron microscopy and scanning probe microscope techniques, there remains a need for an optical imaging method that can uncover not only nanoscopic structures, but also the physical and chemical phenomena occurring on the nanoscale level. In certain examples, SPLM can identify probes that are sensitive to properties of the nanoenvironment, which include, among many others, local pH, temperature, rotational mobility, and proximity to other probes. Thus, SPLM, which combines spectroscopy and super-resolution optical microscopy, provides fundamentally new capabilities in many disciplines, from materials science to the life sciences.

Methods

Optical Setup

In certain examples, the excitation source was a 532 nm diode-pumped solid-state laser with 300-mW maximum output. After passing through a laser clean-up filter (e.g., LL01-532-12.5, Semrock) and further attenuated by a set of ND filters, it was coupled to an inverted microscope body (e.g., Nikon, Eclipse Ti-U), reflected off a dichroic beam splitter (e.g., LPD02-532RU-25, Semrock), and introduced to the sample through the back focal plane of a Nikon CFI apochromat TIRF objective lens (e.g., 100×, 1.49 NA). By shifting the laser beam toward the edge of the TIRF objective with a translation stage, the emerging light reached the sample at near-critical angle of the glass-water interface, thereby illuminating only the fluorophores within a controlled range (usually a micrometer) above the coverslip surface. A 532-nm notch filter (e.g., OD>6, NF01-532U-25, Semrock) was placed at the emission port to reject the reflected laser beam. The fluorescence image was coupled into a Czerny-Turner type monochromator (e.g., SP2150, Princeton Instruments) featuring a blazed dispersive grating (e.g., 150 grooves/mm). The image further divided into a non-dispersed zero-order image and a spectrally dispersed first-order spectral image. By reflecting the zero-order image back to the output port with a silver mirror, both the zero-order and the first-order images can be collected by an EMCCD camera (e.g., proEM, Princeton Instruments) simultaneously.

SPLM Imaging Procedure

In certain examples, the samples were placed on the microscope stage and imaged under a TIRF objective (e.g., Nikon CFI apochromat 100×, 1.49 NA). The 532-nm laser was used to excite fluorescence from Rhodamine, Alaxe Fluor 568, and Mito-EOS 4b. Before acquiring SPLM images, we used relatively low-intensity 532-nm light (e.g., ˜0.05 Wcm⁻²) to illuminate the sample and we recorded the conventional fluorescence image before switching a substantial fraction of the dye molecules to “off” states. We then increased the 532-nm light intensity (e.g., to ˜2 kWcm⁻²) to rapidly switch off the dyes for SPLM imaging. The 405-nm laser was used to reactivate the fluorophores from the dark state back to the emitting state. The power of the 405-nm laser was adjusted to 0.5 Wcm⁻² to maintain an appropriate fraction of the emitting fluorophores. The EMCCD camera acquired images from the monochromator at a frame rate of 50 Hz with field of view of 10×10 μm². Unless specifically noted, 10,000 frames were recorded to generate the super-resolution spectroscopic image.

Imaging Buffer

A standard imaging buffer was freshly made and added to the sample prior to imaging. It contained TN buffer (e.g., 50 mM Tris (pH 8.0) and 10 mM NaCl), an oxygen scavenging system (e.g., 0.5 mg/ml glucose oxidase (e.g., Sigma-Aldrich)), 40 μg/ml catalase (e.g., Sigma-Aldrich) and 10% (w/v) glucose (e.g., Sigma-Aldrich), and 143 mM βWE (e.g., Sigma-Aldrich).

Preparation of Dye-Labeled Actin Monomers

In certain examples, rabbit muscle actin (e.g., Cytoskeleton, Denver, Colo.) was suspended to 0.4 mg/ml in general actin buffer (GAB, e.g., 5 mM Tris-HCl pH 8.0, 0.2 mM CaCl2) supplemented with 0.2 mM ATP and 0.5 mM DTT, and then incubated on ice for 60 min to depolymerize actin oligomers. The solution was centrifuged in a 4° C. microfuge at 14 k rpm for 15 min. Then, 100 μl of the actin solution was transferred from the supernatant to ultracentrifuge tubes. Alexa Fluor 532 Phalloidin solution and Alexa Fluor 568 Phalloidin solution (e.g., Life Technologies, 5 μg/mL in PBS with 3% BSA) were added into the actin solutions, respectively. After incubating at 37° C. for 20 min, the solutions were centrifuged at 100,000×g for 1 h. The top of the supernatant was transferred and diluted to 10⁻⁹M with GAB. For the single-stained imaging sample, the solution containing Alexa Fluor 568 stained actin monomers was deposited onto poly-1-lysine-coated #1.5 coverslips and washed by capillary action with GAB supplemented with 0.2 mM ATP and 0.5 mM DTT. For the dual-color sample, two solutions containing different stained actin monomers were first mixed and then deposited onto coverslips.

Preparation of Rhodamine-Labeled Microtubules

In certain examples, Rhodamine-labeled microtubules were assembled in vitro by using lyophilized Rhodamine-conjugated tubulin (e.g., Cytoskeleton, Denver, Colo.) incubated at 37° C. for 20 min in general tubulin buffer (GTB, e.g., 80 mM PIPES pH 6.9, 2 mM MgCl2, and 0.5 mM EGTA) supplemented with 10% glycerol and 1 mM GTP (e.g., Cytoskeleton, Denver, Colo.) at a final concentration of 4 mg/ml. The microtubules were stabilized by incubating 25 μM taxol (e.g., Enzo Life Sciences, Farmingdale, N.Y.) for an additional five minutes at 37° C. For imaging, the microtubules were deposited onto poly-1-lysine-coated #1 coverslips and washed by capillary action with 100 μM GTB supplemented with 20 μM paclitaxel (taxol) and 1 mM GTP.

Preparation for Cellular Imaging

In certain examples, COS-7 cells (e.g., ATCC) were grown in DMEM (e.g., Gibco/Life Technologies) supplemented with 2 mM L-glutamine (e.g., Gibco/Life Technologies), 10% fetal bovine serum (e.g., Gibco/Life Technologies), and 1% penicillin (e.g., 10,000 IU/mL)/streptomycin (e.g., 10,000 μg/mL) (e.g., Gibco/Life Technologies) at 37° C. with 5% CO₂. The cells were transiently transfected with mEOS 4b-Tomm20 (e.g., Michael Davidson) using BioRad Gene Pulser XCell, and were plated on 18-mm diameter #1.5 glass coverslips. After 48 hours, the cells were fixed in 0.8% formaldehyde and 0.1% gluteraldehyde in PBS for 5 min at room temperature, reduced with 1% sodium borohydride for 7 min, and then further reduced in 1 mM lysine. Followed by extraction in 0.2% tween-20 in PBS for 5 min, the cells were rinsed with PBS and incubated in a blocking buffer (e.g., 3% BSA (e.g., Sigma) and 1% NGS in PBS) for 30 min at room temperature. The buffer was aspirated and the cells were incubated with mouse anti-α-tubulin antibody (e.g., Sigma, 1:1,000 dilution in PBS, 3% BSA) at 37° C. for 20 min. The cells were soaked in PBS five times in 10-min intervals to rinse off the primary antibody solution. Goat-anti mouse Alexa Fluor 568 solution (e.g., Life Technologies, 5 μg/mL in PBS with 3% BSA) was added to the coverslips and the cells were incubated at 37° C. for 20 min. Afterward, the samples were rinsed in PBS for 1 h (e.g., 6-7 changes) and stored in PBS at 4° C. until imaging. Prior to imaging, the sample was briefly washed once with PBS and then immediately mounted for SPLM imaging. Imaging buffer (e.g., ˜4 μl) was dropped at the center of a freshly cleaned glass slide, and the sample on the coverslip was mounted on the glass slide and sealed with dental cement.

Examples of Sequencing by Fingerprinting

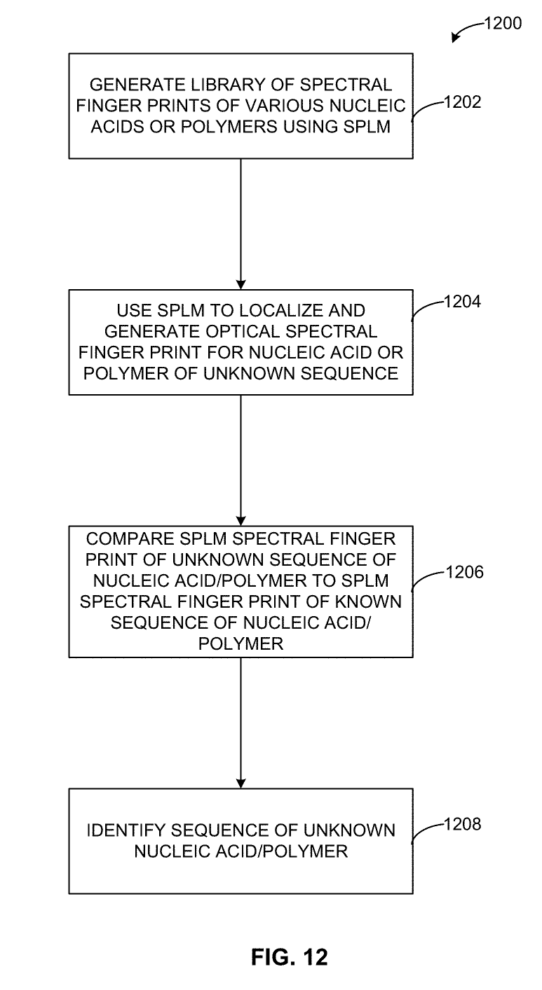

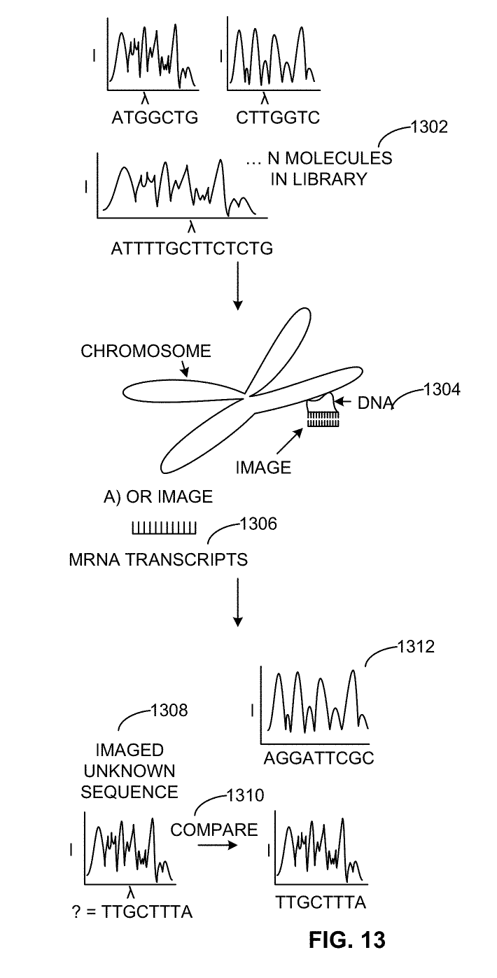

FIG. 12 illustrates an example flow diagram of a method to sequence nucleic acids and/or polymers by fingerprinting. At block 1202, a library of spectral fingerprints of various nucleic acids and/or polymers is generated using SPLM. For example, FIG. 13 shows molecules 1 to n in a library 1302.

At block 1204, SPLM is used to localize and generate an optical spectral fingerprint for a nucleic acid or polymer of unknown sequence. For example, as shown in FIG. 13, a chromosome can be analyzed using SPLM to generate a DNA image 1304 and/or image of RNA transcripts 1306.

At block 1206, the SPLM spectral fingerprint of the unknown sequence of nucleic acid or polymer is compared to the SPLM spectral fingerprint of a known sequence of nucleic acid or polymer. For example, as shown in FIG. 13, the imaged unknown sequence of nucleic acid/polymer 1308 is compared 1310 (e.g., based on the corresponding SPLM spectral fingerprint) to a known sequence of nucleic acid/polymer 1312 from the library 1302.

At block 1208, the unknown sequence of nucleic acid or polymer is identified based on the SPLM spectral fingerprint comparison.

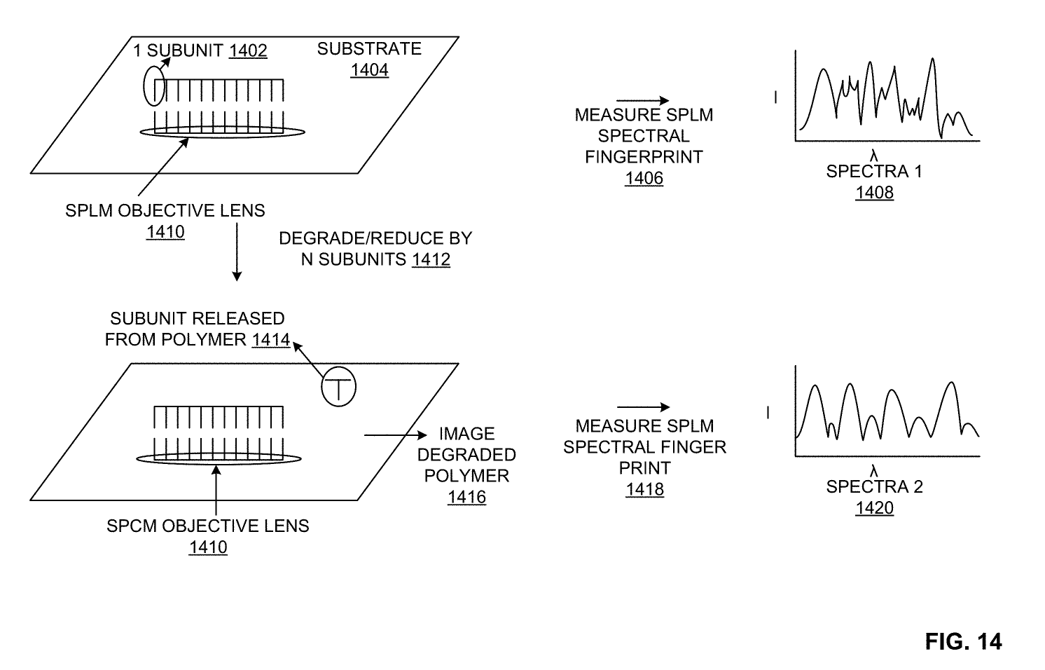

FIG. 14 illustrates an example of sequencing by degradation using SPLM. As shown in the example of FIG. 14, a polymerized nucleic acid and/or a polymer can be analyzed based on a selected subunit 1402 on a substrate 1404. A SPLM spectral fingerprint 1408 can be measured 1406 using the subunit 1402. Additionally, an SPLM objective lens 1410 can be used to degrade and/or otherwise reduce the sample by n subunits 1412 such that a subunit is released from the polymer/acid 1414. The degraded polymer/acid is imaged 1416, and an SPLM spectral fingerprint 1420 is measured 1418 using the degraded polymer/acid image. The spectra 1420 can be compared to the spectra 1410 to determine an identity of the released subunit 1414 of the polymer/nucleic acid.

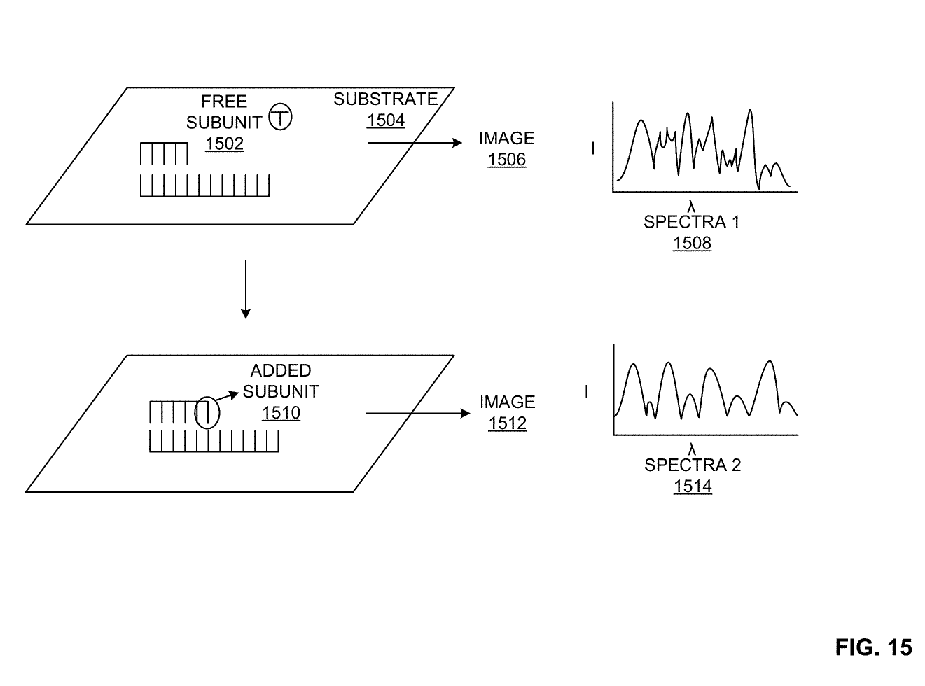

FIG. 15 illustrates an example of sequencing by synthesis. As shown in the example of FIG. 15, a subunit can be freed 1502 from a substrate 1504 and imaged 1506 using SPLM to form a first spectral fingerprint 1508. A subunit 1510 can be added to the substrate 1504 and imaged 1512 to form a second spectral fingerprint 1514. The first spectral fingerprint 1508 can be compared to the second spectral fingerprint 1514 to identify the added subunit 1510.

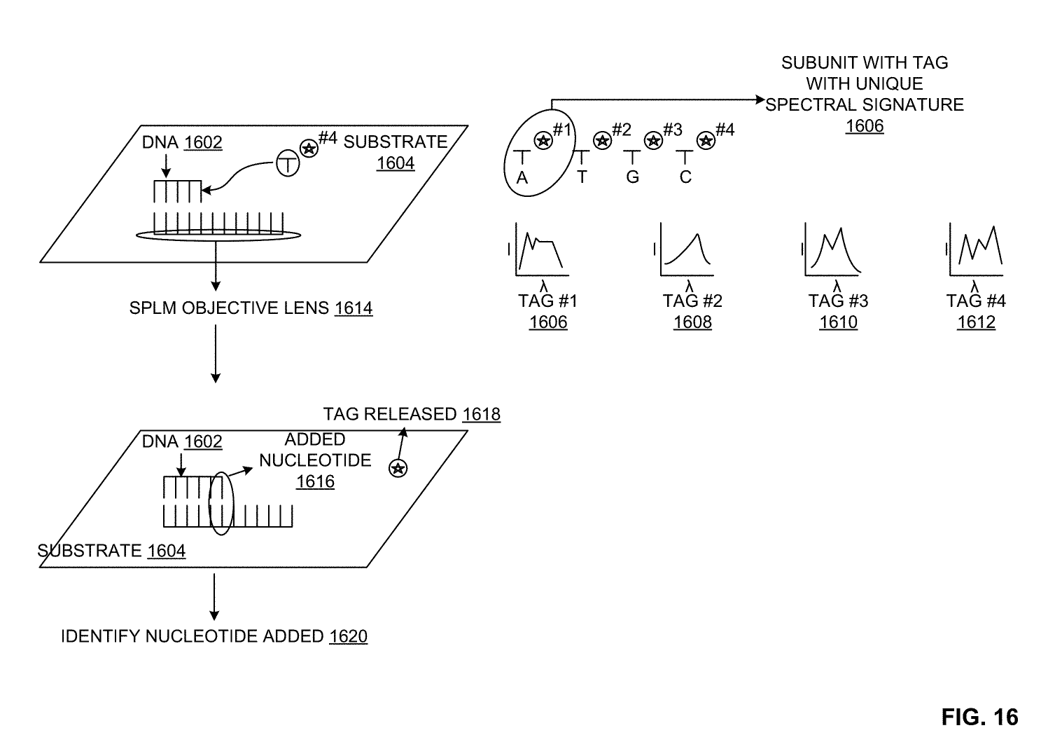

FIG. 16 illustrates another example of sequencing by synthesis. As shown in the example of FIG. 16, a DNA sample 1602 positioned on a substrate 1604 includes a subunit with a tag having a unique spectral signature 1606. Each tag 1606-1612 has an associated spectral signature. Using an objective lens 1614, a labelled nucleotide 1616 is added to the DNA sample 1602 on the substrate 1604, and, upon nucleotide addition, a tag is released 1618 from the substrate 1604. SPLM can be used to image data from the tag release 1618 to form a spectral fingerprint to compare to known tag spectral data. Based on the comparison, the added nucleotide 1616 can be identified 1620.

Examples of Identifying an Analyte of Interest

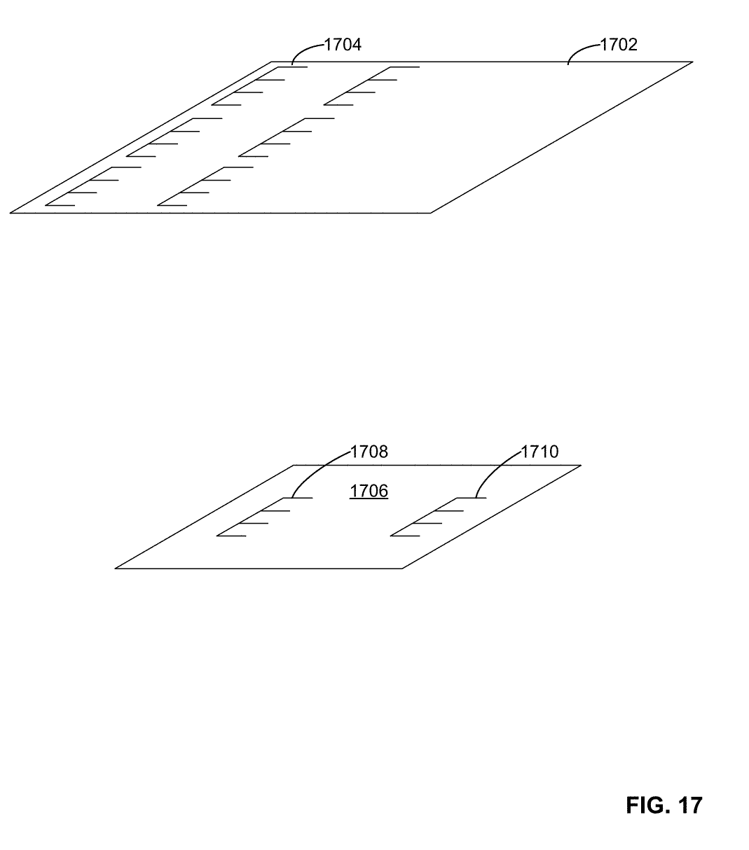

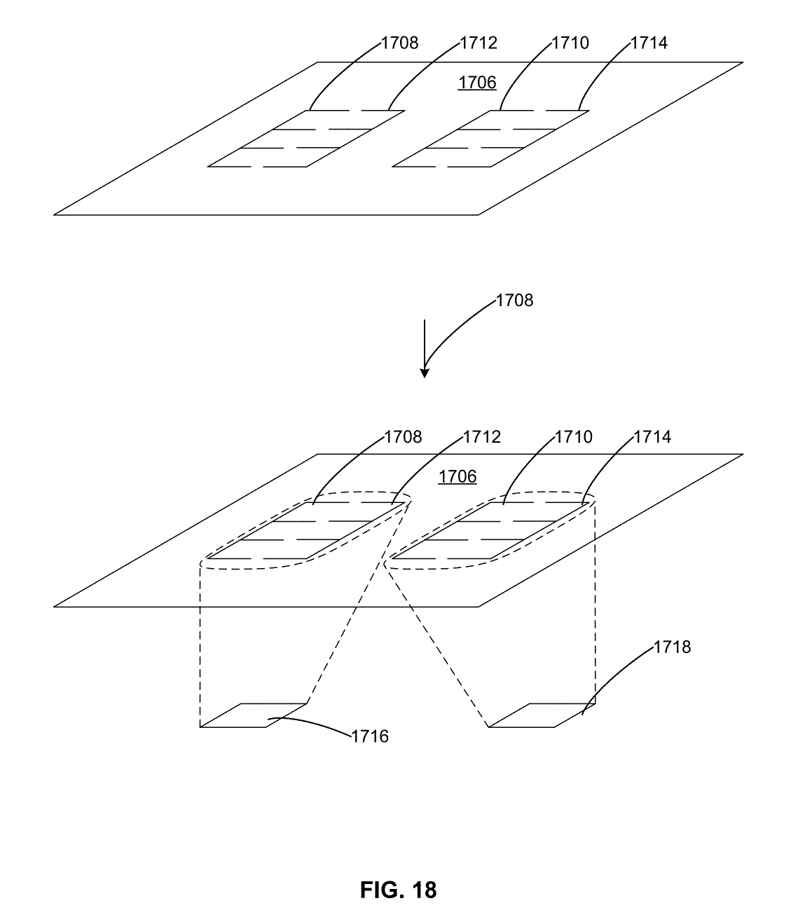

FIG. 17 illustrates an example substrate 1702 including a plurality of probes 1704. A subset 1706 includes a first DNA oligo sequence 1708 and a second DNA oligo sequence 1710. As shown in FIG. 18, a first RNA analyte 1712 is added to the first DNA sequence 1708, and a second RNA analyte 1714 is added to the second DNA sequence 1710. The combinations are imaged 1716, 1718. Localization can be observed based on a number of photon counts, and a spectral profile of the DNA-RNA can be observed by probing the analyte complex.

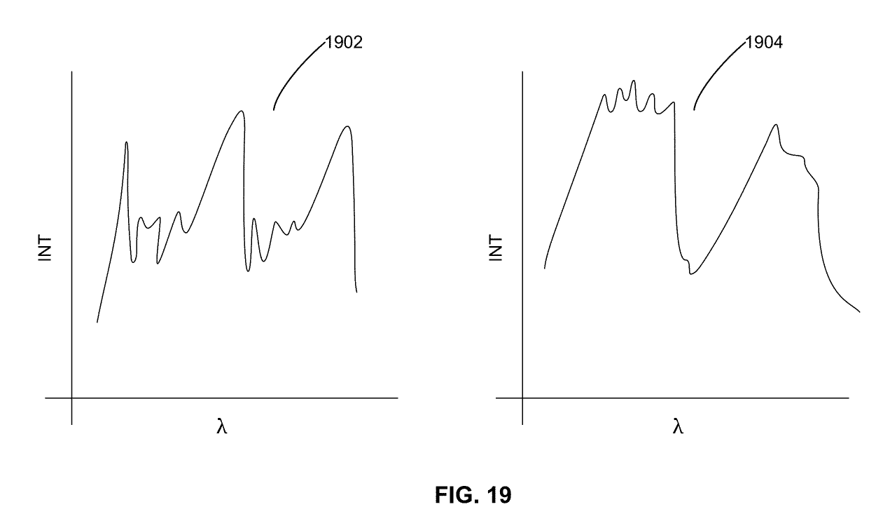

Example spectral profiles 1902, 1904 based on the images 1716, 1718 are shown in FIG. 19. From the spectral profiles 1902, 1904, a number of spectral profiles associated with the first spectral profile 1902 can be quantified, and a number of spectral profiles associated with the second spectral profile 1904 can also be quantified. The spectral profiles 1902, 1904 can be used to identify the analytes 1712, 1714.

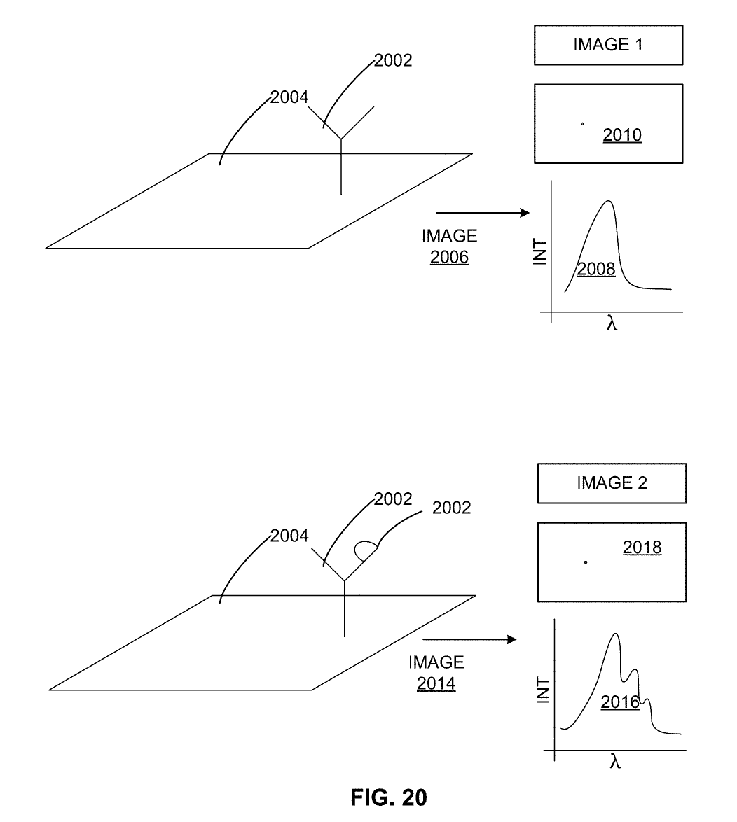

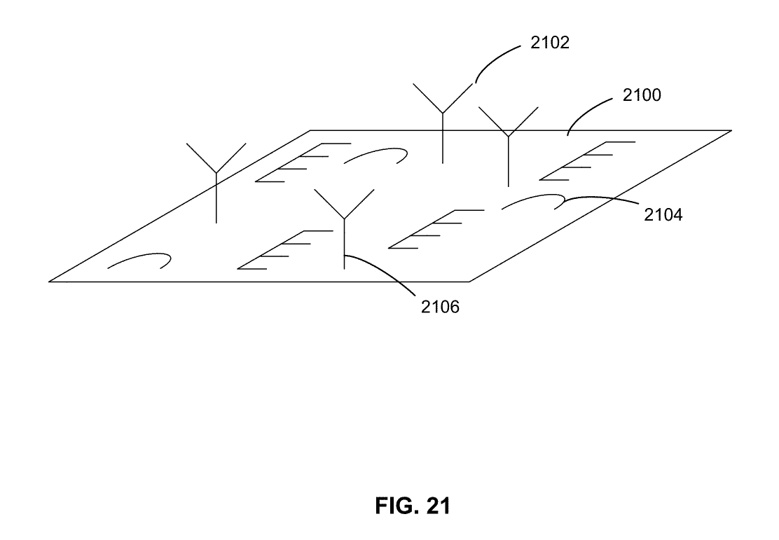

FIG. 20 depicts a probe (antibody) 2002 on a substrate 2004 that can be imaged 2006 to generate a spectral fingerprint 2008 (top down view shown in the inset 2010). An analyte 2012 can be bound to the antibody probe 2002, which can then also be imaged 2014 to generate a spectral fingerprint 2016 (top view shown in the inset 2018). A change in spectral pattern indicates a presence or absence of an identifiable analyte. Changes between spectral images 2008 (2010) and 2016 (2018) can be analyzed to detect, quantify, and characterize the analyte 2012 and/or the probe 2002, for example. As shown in the example of FIG. 21, an array 2100 can include multiple types of probes including a nucleic acid probe 2102, a chemical probe 2104, an antibody probe 2106, etc.

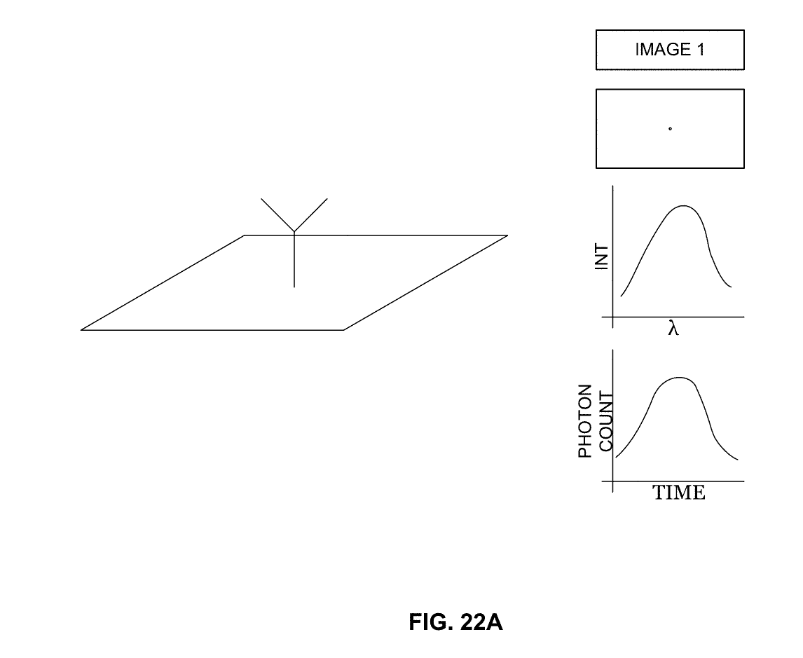

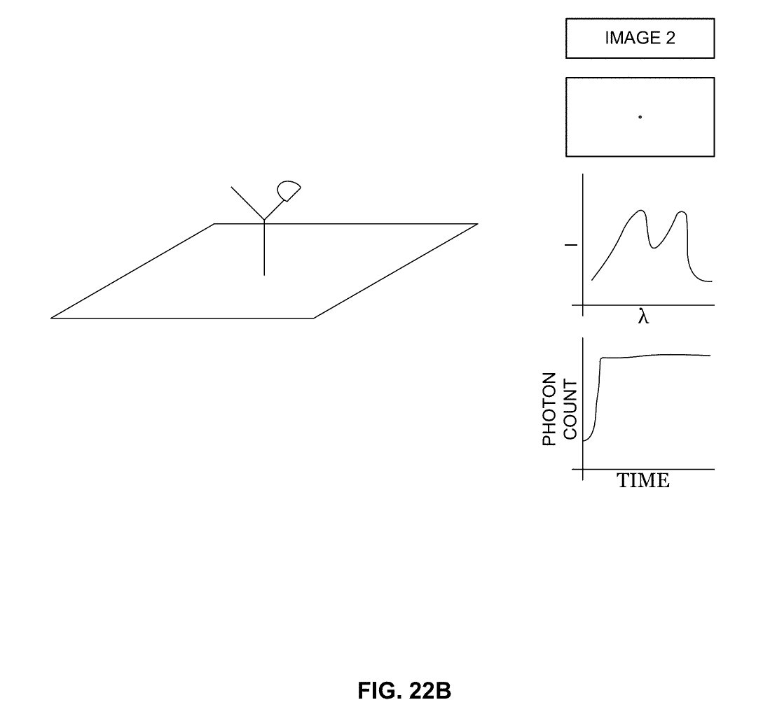

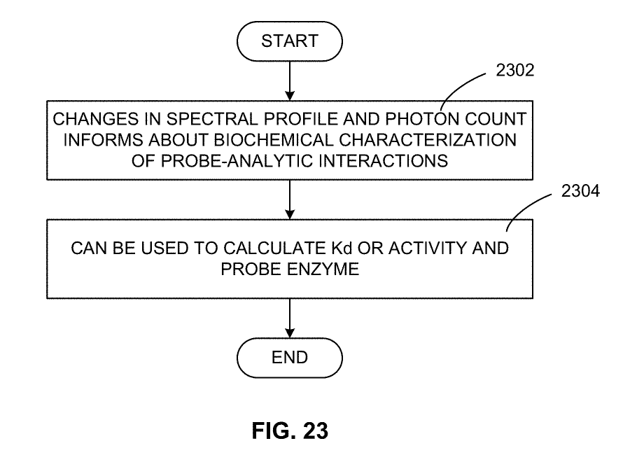

As illustrated in FIGS. 22a-22b, images can be compared based on top down image, spectra, and photon count over time. As shown in the example of FIG. 23, first and second images can be compared to determine, at 2302, changes in spectral profile and photon count to inform biochemical characterization of probe-analyte interaction. At 2304, the biochemical interaction can be used to calculate a dissociation constant, Kd, or activity of a probe enzyme, for example.

Examples of Detecting, Selecting, and Sorting Cells

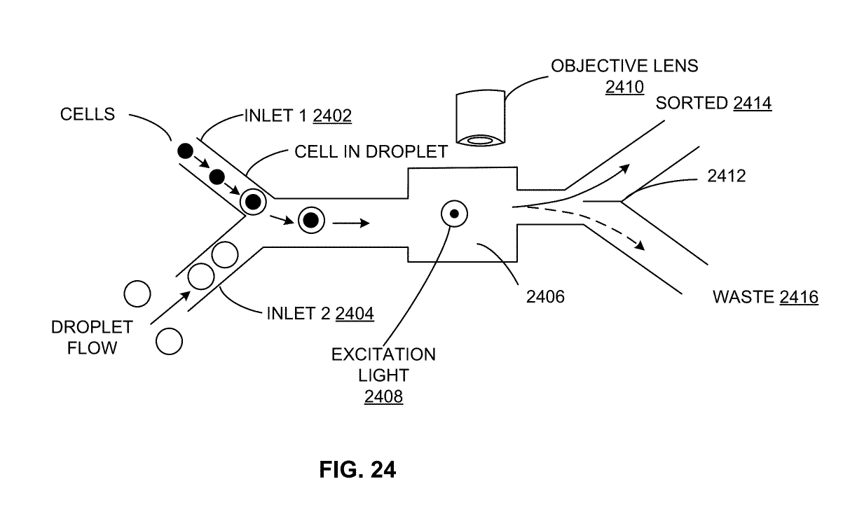

A cell sorting apparatus is constructed (FIG. 24) to encapsulate cells into micro droplets which can be manipulated. The micro droplet material may include magnetic particles, which can be used to suspend the droplet, fix the droplet, or capture the droplet in precise locations, including the contents of the droplet. In this example, cells to be sorted are unlabeled and allowed to enter flow chamber 1 2402 which feeds into a common channel also fed by flow chamber 2 2404. Flow chamber 2 2404 contains micro droplet material, what when combined with the cells, creates encapsulates the cells. The encapsulated cells are flowed into an imaging chamber 2406 with an excitation light 2408 and an objective lens 2410. In some examples, a mechanism to capture the cells on imaging substrate is used. In this example, a magnetic field is applied to capture the cells on the imaging substrate.

SPLM imaging is then used to image a variety of biomolecules in the cell, thereby creating a signal finger print. The signal finger print may include imaging patterns of biomolecules, including the structures of various subcellular structures such as microtubules, chromosomes, nucleus, membranes, ribosomes etc. Fingerprinting is also generated by the localization and identification specific sequences or identity of certain proteins. In this example, SPLM is used to identify and quantify specific RNAs. Based on individual cell spectral fingerprints from SPLM imaging, different cells may be sorted based on fingerprints.

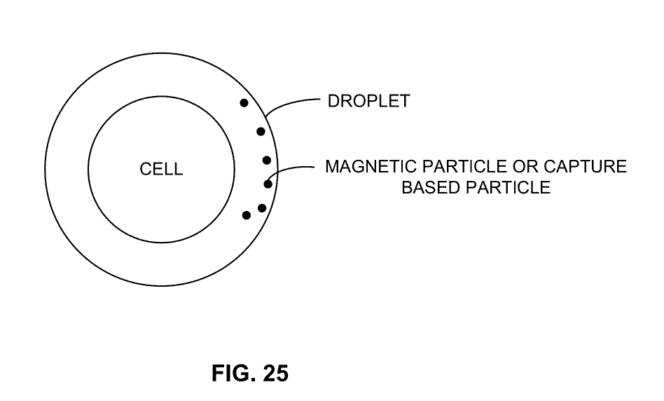

After the fingerprint is generated, the cell is released from the substrate and is flowed to a specific container based on the fingerprint. For example, the flow can be gated 2412 based on a signal from the SPLM characterization of the cell to be sorted 2414 or sent to waste 2416. An example cell is shown in FIG. 25.

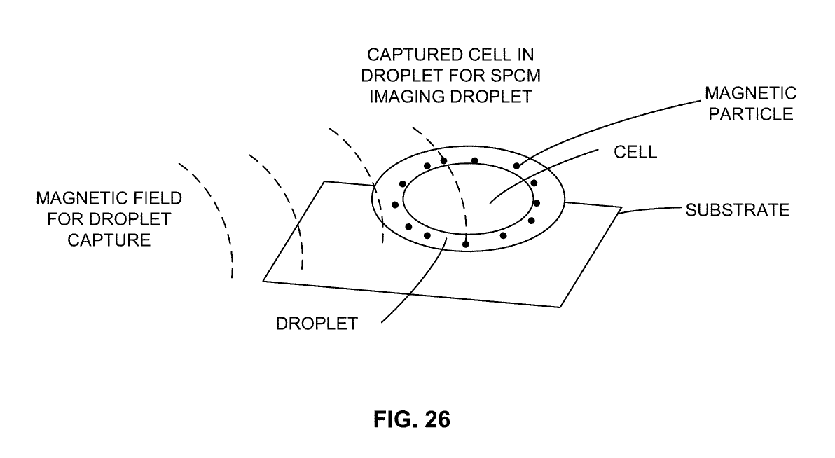

FIG. 26 illustrates a captured cell in a droplet for SPLM imaging. As shown in the example of FIG. 26, a cell in a droplet on a substrate includes a magnetic particle(s), and a magnetic field is applied for droplet capture.

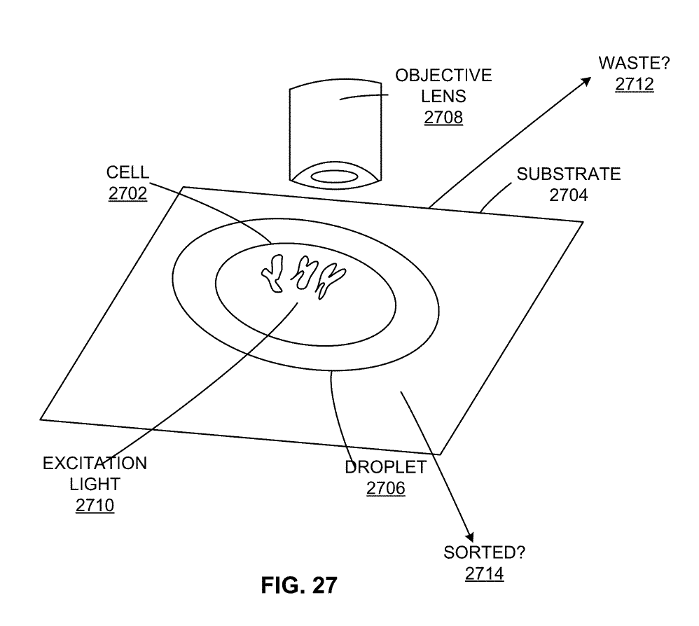

FIG. 27 illustrates an example apparatus for cell analysis including a cell 2702 on a substrate 2704 in a droplet 2706 with respect to an objective lens 2708 and an excitation light 2710. SPLM imaging is used to resolve subcellular structures and nucleic acid sequencing for cell selection criteria (e.g., waste 2712 or sorted 2714).

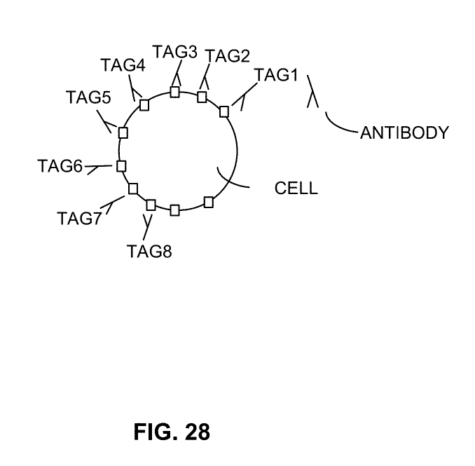

FIG. 28 illustrates an example cell sorting based on membrane markers. Each tag has a unique spectral profile to enable sorting of cells.

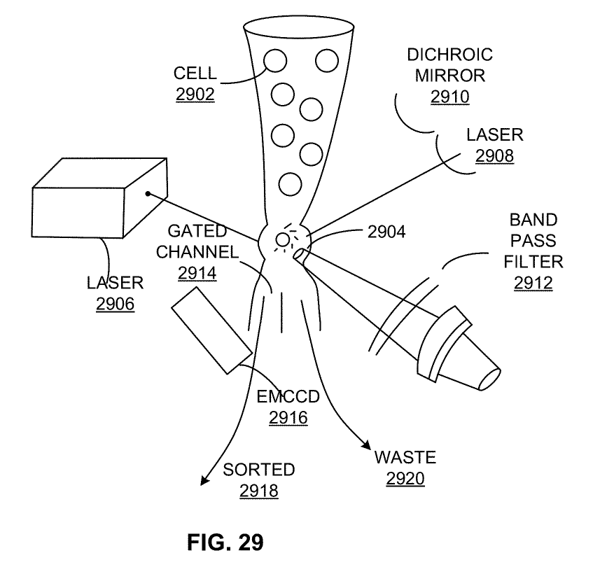

FIG. 29 illustrates an example system 2900 for sorting based on localization and spectral profile with potentially unlimited channel (e.g., not limited to four channels, 4 color labeling, etc.). As shown in the example of FIG. 29, cells 2902 are provided into a chamber 2940 and illuminated using a first laser 2906 and a second laser 2908 with a dichroic mirror 2910. The lasers 2906, 2908 and mirror 2910 work with a bandpass filter 2912 through a gated channel 2914 to image the cells 2902 using the EMCCD 2916. Cells are then sorted 2918 or routed to waste 2920, for example.

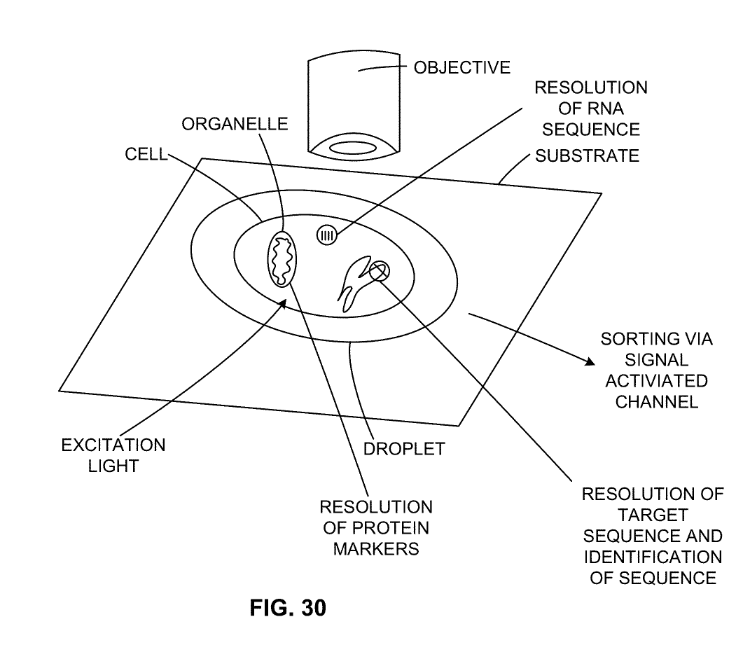

FIG. 30 illustrates another example apparatus for cell analysis including an organelle in a cell in a droplet on a substrate under an objective lens in view of an excitation light. The example apparatus of FIG. 30 facilitates resolution of protein marker(s), resolution of RNA sequence(s), resolution of target sequence and identification of sequence, sorting via signal activated channel, etc.

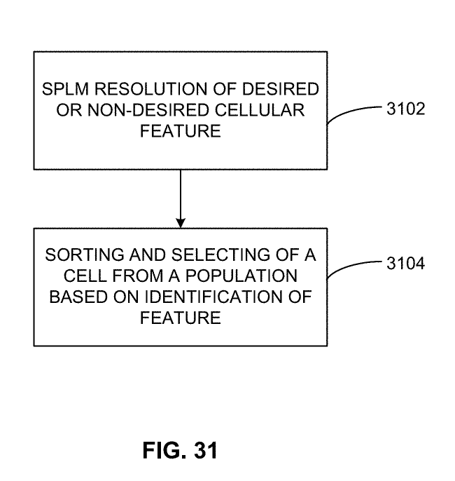

FIG. 31 illustrates a flow diagram of an example method for SPLM resolution and cell analysis. At block 3102, SPLM resolution of desired or non-desired cellular features is obtained (e.g., using FIGS. 24-30). At block 3104, a cell is sorted and selected from a population based on the identification of feature(s) from block 3102.

Examples of Image Labeling

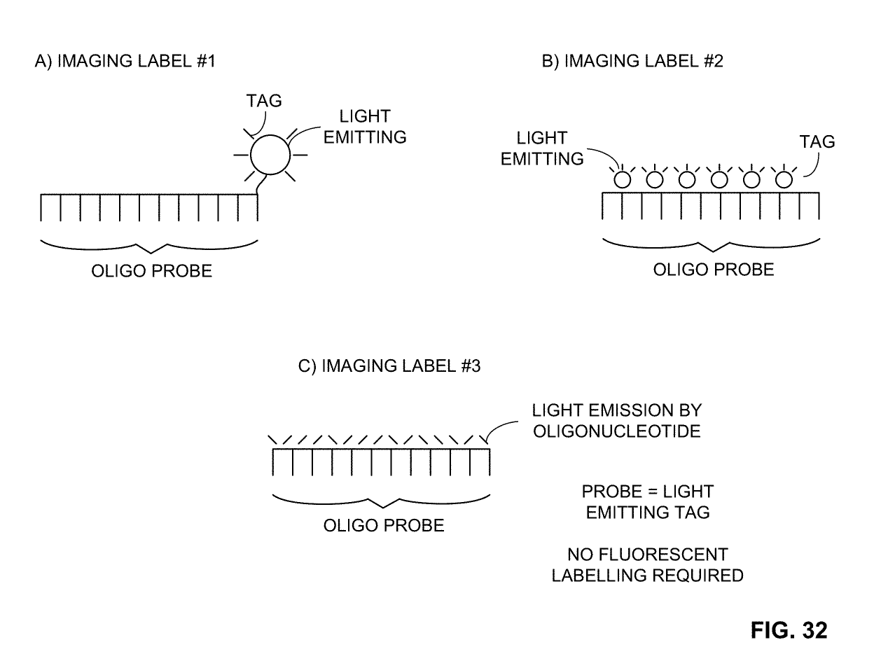

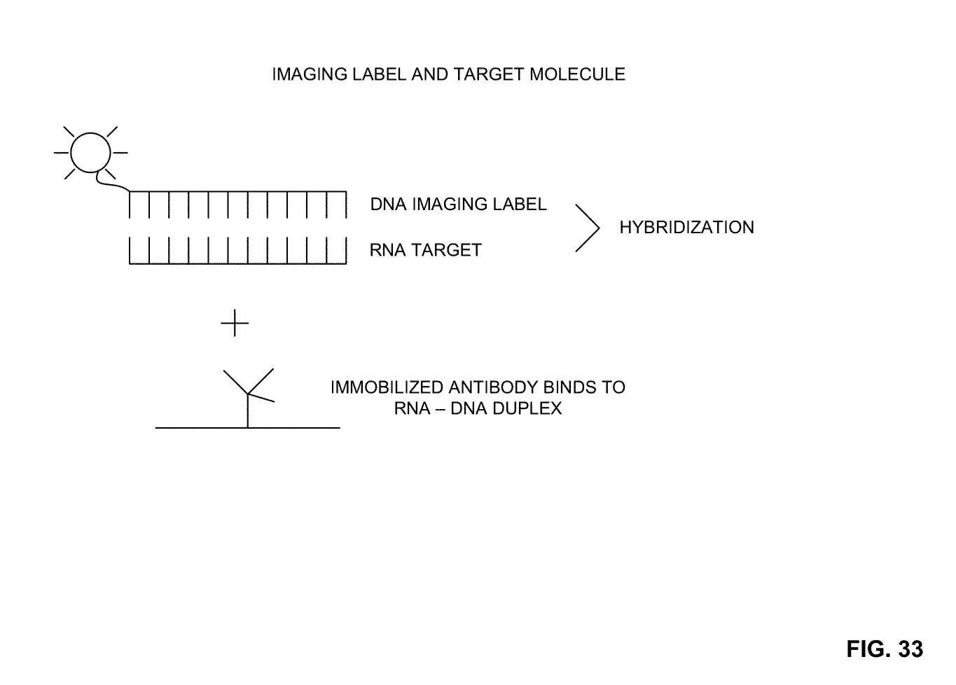

In certain examples, imaging labels can be applied to one or more probes including nucleic acid sequence(s), immobilization element(s), tag(s), etc. FIG. 32 shows an example set of imaging labels 1, 2, 3 including DNA oligo sequences, tags, and light emitting oligonucleotides forming probes for analysis. FIG. 33 shows an example of imaging labeling with a target molecule. In the example of FIG. 33, a DNA imaging label is hybridized with a RNA target and combined with an immobilized antibody which binds to the RNA-DNA duplex.

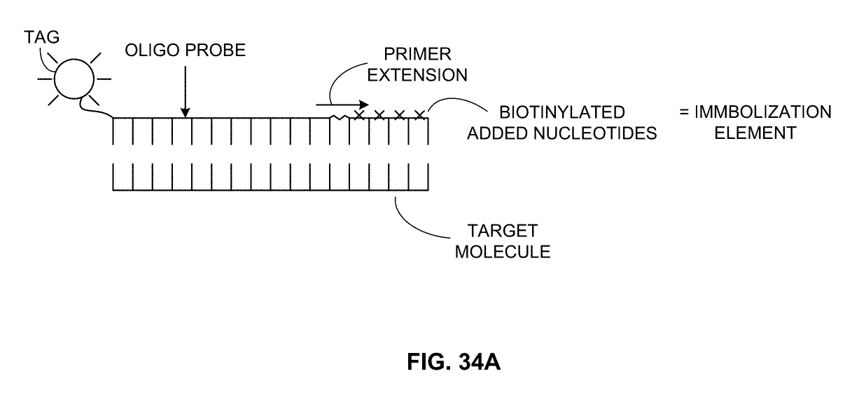

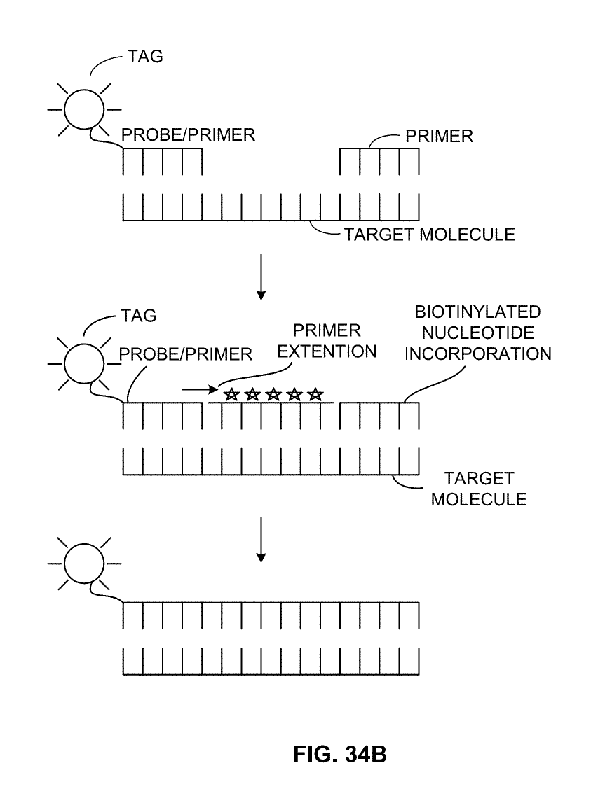

FIG. 34A shows an example with a target molecule labeled using an immobilization element including a tag, an oligo probe, a primer extension, and biotinylated added nucleotides. As shown in FIG. 34B, a probe/primer with a tag is bound to a target molecule. A primer extension is then added along with biotinylated nucleotide incorporation. Ligation results in a combined probe molecule.

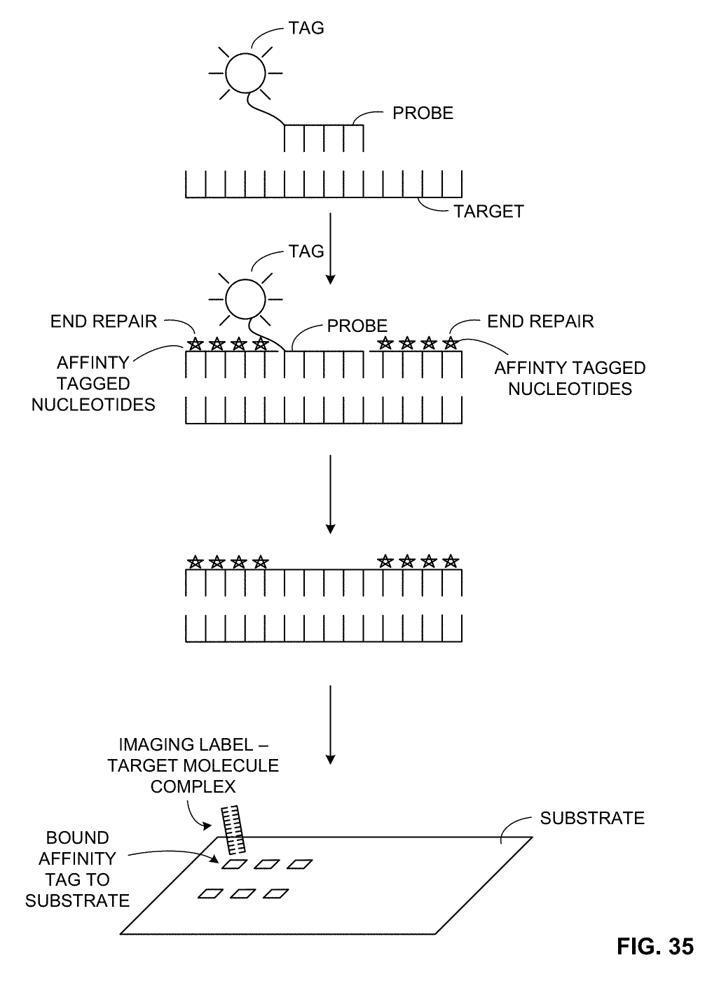

Similarly, FIG. 35 shows a probe and tag being bound to a target. End repair is used to add affinity tagged nucleotides to both ends of the probe to form a combined target that can be used on a substrate as an imaging label—target molecule complex with an affinity tag bound to the substrate.

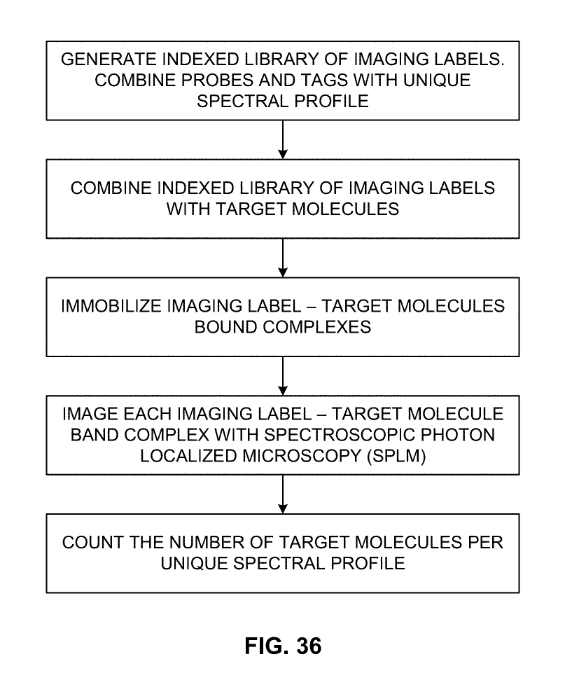

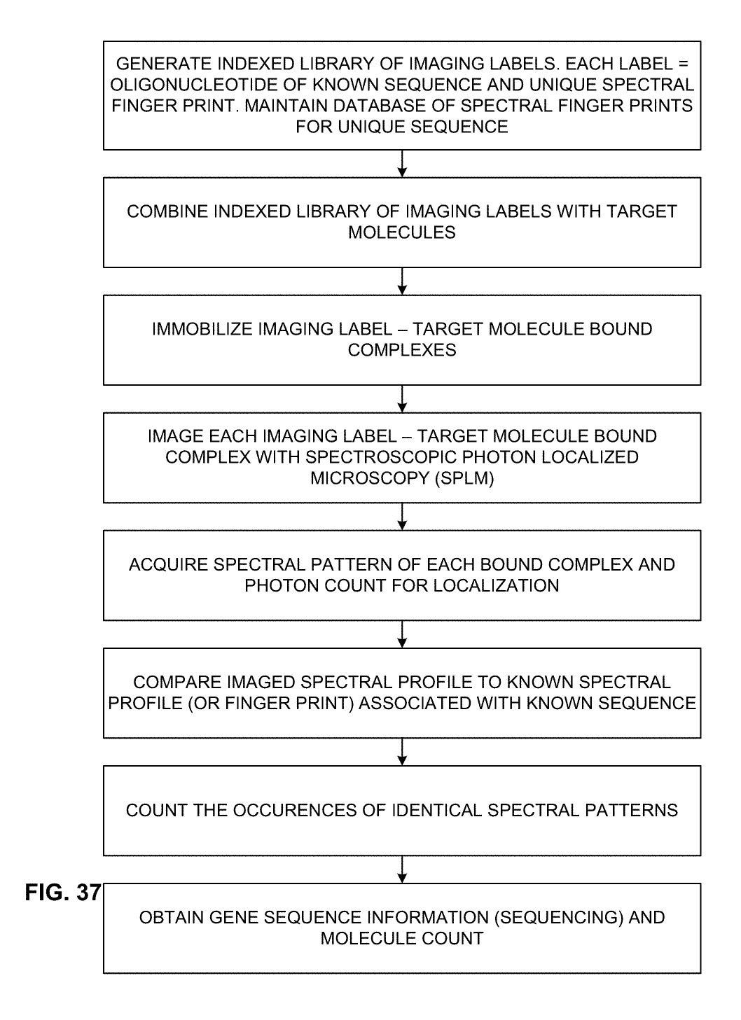

FIG. 36 illustrates a flow diagram of a method for imaging label and analysis. At block 3602, an indexed library of imaging labels is generated combining probes and tags with unique spectral profile. At block 3604, the indexed library of imaging labels is combined with target molecules. At block 3606, imaging label-target molecules are immobilized into bound complexes. At block 3608, each imaging label—target molecule bound complex is imaged with spectroscopic photon localized microscopy (SPLM). At block 3610, a number of target molecules per unique spectral profile is counted.

FIG. 37 illustrates a flow diagram of a method for imaging label and analysis. At block 3702, an indexed library of imaging labels is generated. Each label—oligonucleotide of known sequence is associated with a unique spectral fingerprint. A database of spectral fingerprints can be maintained for unique sequences.

At block 3704, the indexed library of imaging labels is combined with target molecules. At block 3706, imaging label-target molecules are immobilized as bound complexes. At block 3708, each imaging label—target molecule bound complex is imaged with SPLM. At block 3710, a spectral pattern of each bound complex is acquired as well as a photon count for localization. At block 3712, the imaged spectral profile is compared to known spectral profile(s) (or fingerprint(s)) associated with known sequence(s). At block 3714, occurrences of identical spectral pattern(s) are counted. At block 3716, gene sequence information (sequencing) is obtained along with molecule count.

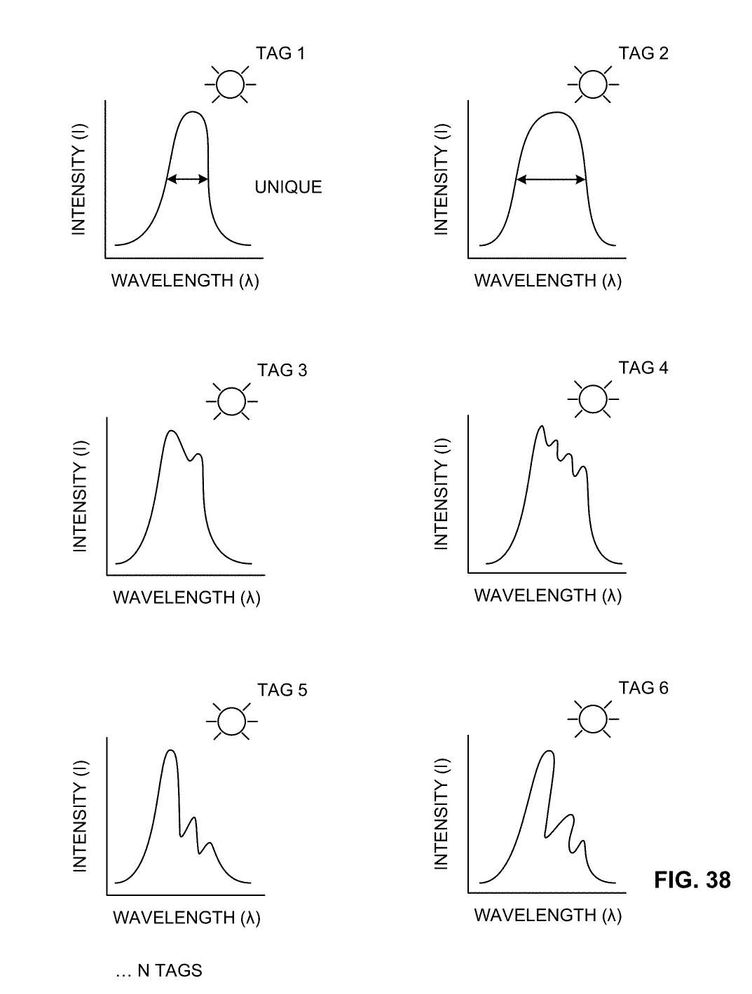

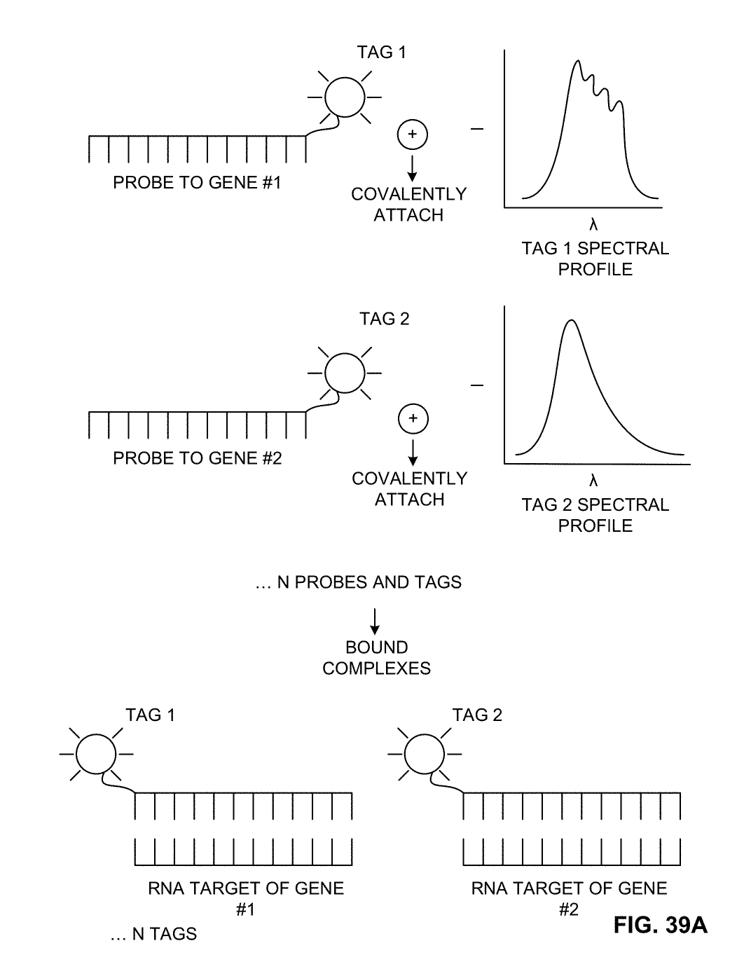

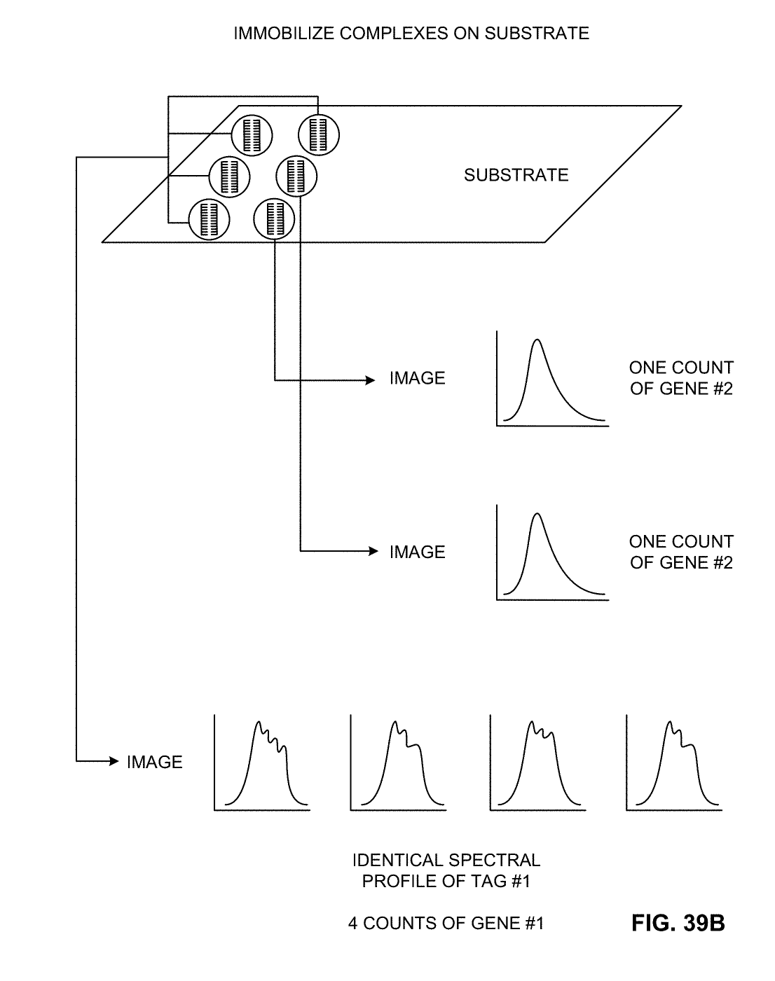

FIG. 38 depicts example spectral profiles reflecting differences in spectral curve shape and size. FIGS. 39a-b show an example of constructing an indexed library and immobilizing complexes on a substrate having a plurality of imaging label target molecule complexes.

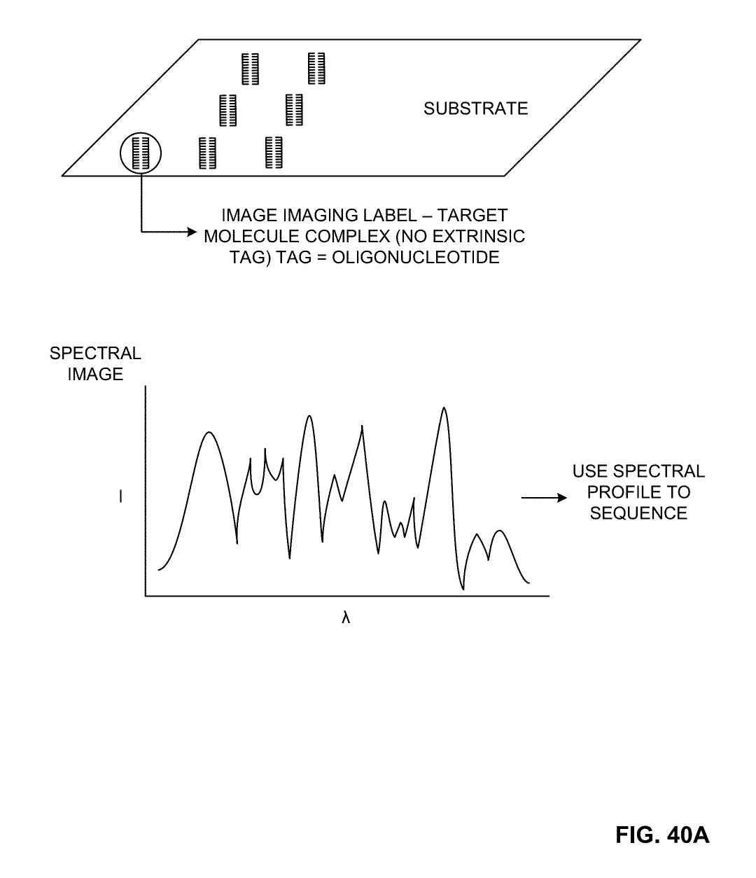

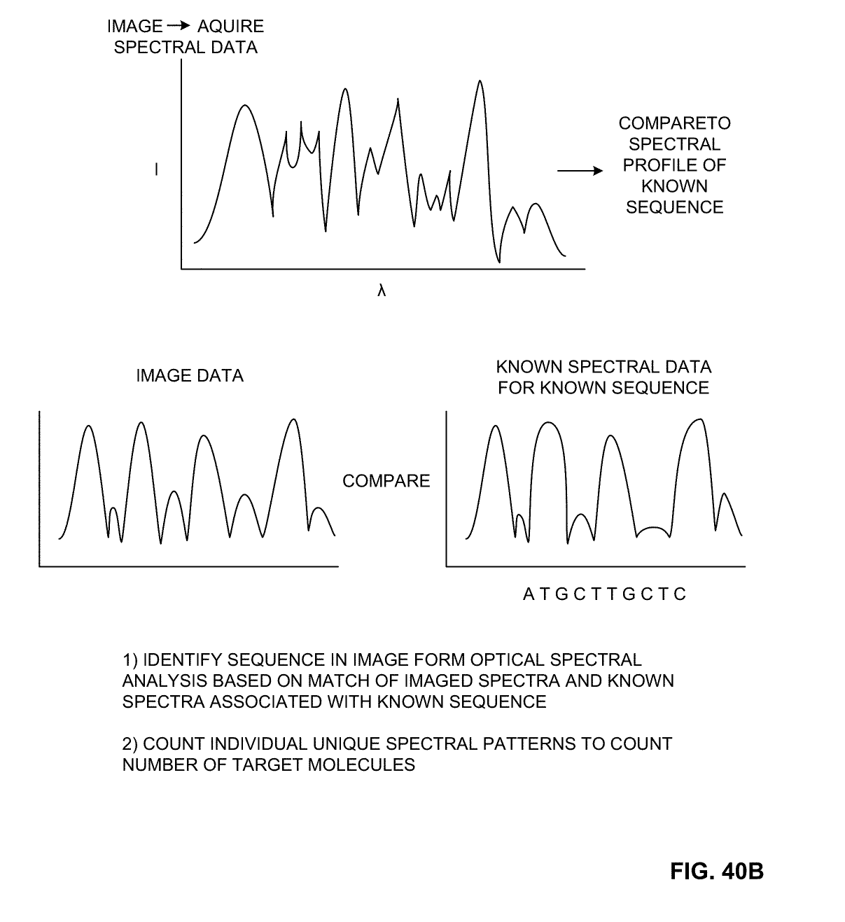

FIGS. 40a-b illustrate an example methodology to image imaging label—target molecule complexes (without extrinsic tag) using oligonucleotides as tags and comparing associated spectral profiles to identify a sequence in an image from optical spectral analysis based on a match of imaged spectra and known spectra associated with known sequence(s). Individual unique spectral patterns can be counted to count a number of target molecules.

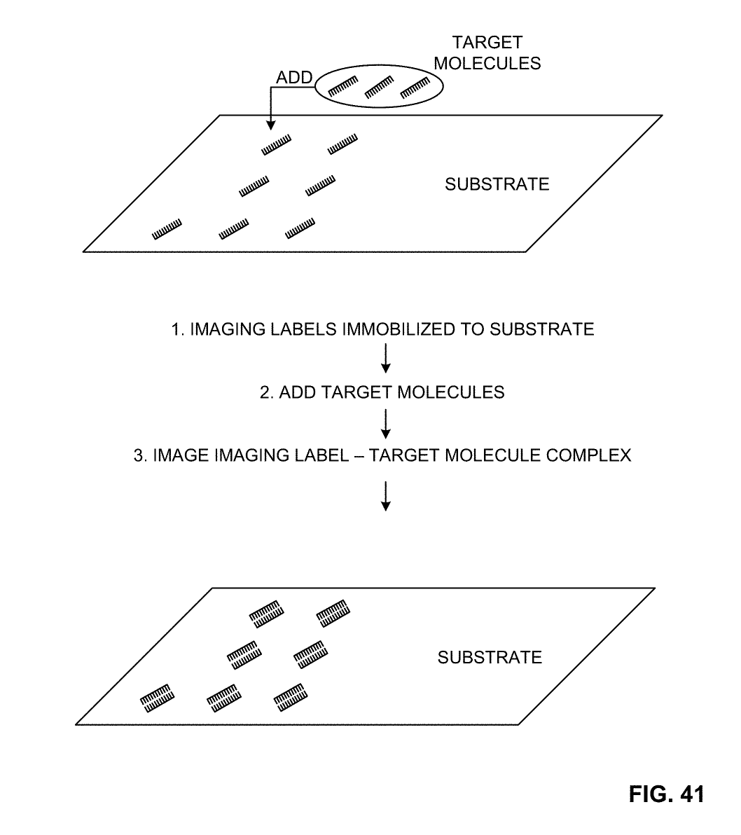

FIG. 41 shows another example substrate with imaging labels and target molecules immobilized to a substrate for analysis.

Examples of Pathogen Characterization

Tuberculosis (TB) infected 9 million people and caused 1.5 million deaths worldwide in 2013. Treatment for TB involves the use of multiple drugs over a 6 to 9 month period. If treatment is improperly administered or incomplete, drug-resistant strains of TB can develop. Resistance is classified based on bacteria susceptibility to first and second line drugs. Resistance to two or more first line drugs is classified as multidrug-resistant TB (MDR-TB) while resistance to second-line drugs is associated with extensively drug-resistant TB (XDR-TB). Rifampicin and isoniazid are the major first line drugs used to treat TB and one or both of these drugs usually accounts for the type of MDR-TB exhibited. Drug-resistant TB is a major concern since it requires treatment with more expensive, toxic drugs over a period of about 2 years. Since TB is an airborne disease and can be easily transmitted, it is vital to prevent the spread of drug resistant forms. In 2013, the World Health Organization (WHO) estimated 480,000 new drug-resistant cases, however, only 136,000 confirmed due to limited access to appropriate tests for drug-resistance. In order to prevent the spread of drug-resistant strains of the bacteria, there needs to be rapid diagnosis of the specific transmitted TB strain so the patient can be placed on the most effective form of treatment.

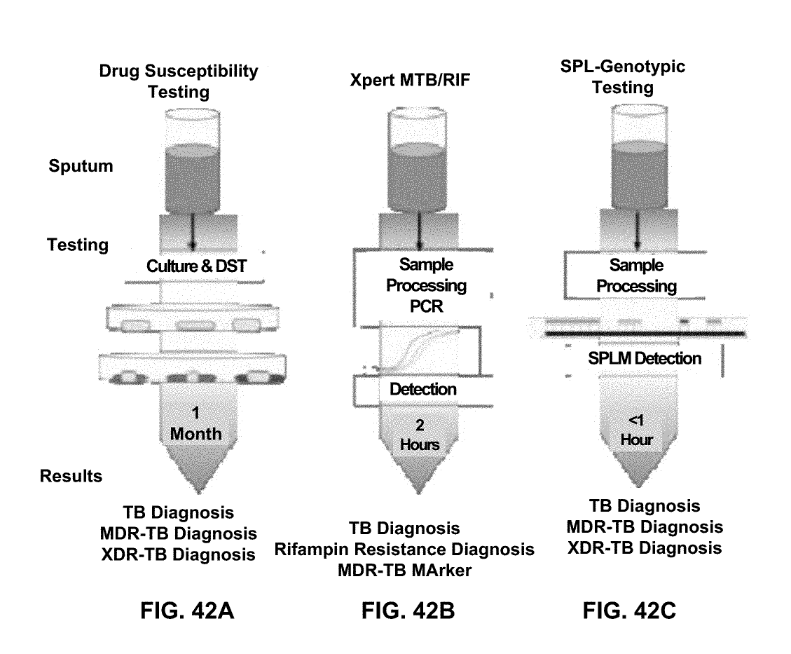

Drug susceptibility testing (DST) shown in FIG. 42a, is the most accurate way to determine the type of treatment a patient should receive. However, culture takes 4-8 weeks and requires trained personnel and increased lab safety levels to prevent self-infection, making DST impractical for initial diagnosis especially in limited-resource clinics. Initial diagnosis for drug resistance is done using Cepheid's Xpert® MTB/RIF assay shown in FIG. 42b. This test has been used to both screen for TB and detect rifampin resistant TB. In areas where drug resistance is high, rifampin resistance is also linked to isoniazid resistance and is therefore used as the criteria for MDR-TB diagnosis. However, it has been found that rifampin resistance is a poor indicator of isoniazid resistance in about ⅓ of countries and subnational regions where incidence MDR-TB is low. In order to reduce the incidence of XDR-TB, second-line drugs are reserved for cases when TB is resistant to rifampin, isoniazid and their derivatives which make up most of the first-line drugs. Therefore, incorrectly prescribing second line drugs can increase the risk of XDR-TB developing and also requires the patient to undergo treatment which is longer, more expensive and uses drugs with increased risk of serious side-effects. Additionally, the Xpert® MTB/RIF assay is only 67% sensitive in cases where the TB results in negative results by sputum smear microscopy despite positive mycobacterial sputum culture. Smear microscopy detects Acid Fast Bacilli in whole sputum with 54% sensitivity and 77% specificity. Though this test performs poorly it is currently one of the most affordable TB tests and its widespread use has contributed to the slow decline of the incidence of TB. The poor performance of smear microscopy is a major concern since 24-61% of smear negative/culture positive TB cases are from individuals with HIV, who make up 13% of new cases of TB and contribute to 25% of TB related deaths. The gaps in the ability to properly diagnose TB and MDR-TB presents the opportunity to develop an improved platform to screen for TB as well as detect the specific strain of TB. We propose the development of a spectroscopic photon localization genotypic testing (SPL-Gene Testing) platform which can be used no identify mutations in targeted genes. The goal of this project is to optimize and test the SPLM detection system which would serve as the basis of the SPL-Genotypic Testing platform.

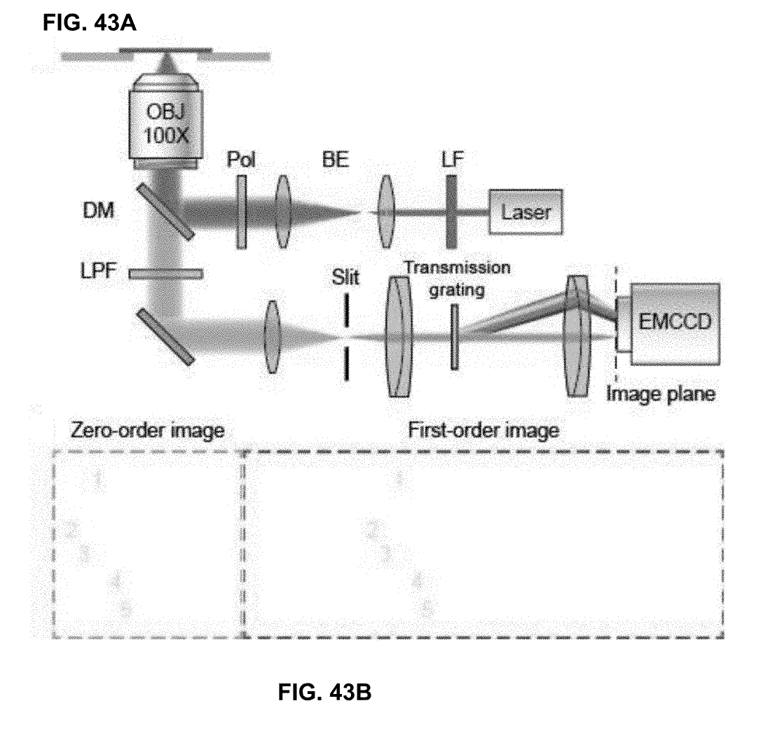

SPLM platform development and validation: The original SPLM system developed by the Zhang lab uses a 532 nm diode pumped solid-state laser with 330-mW maximum output. The laser beam is filtered using a narrow bandpass filter (LL01-532-12.5, Semrock) and ND filters used to further attenuate the beam. The attenuated beam is then coupled to an inverted microscope body (Nikon, Eclipse To-U), reflected using a dichroic beam splitter (LPD02-532RU-25, Semrock) and is used to illuminate the sample at the back focal plane of a Nikon CFI apochromatic total internal reflection (TIRF) objective lens (100×, 1.49 NA). The light beam could be adjusted to perform TIRF imaging, which significantly improves the signal to noise ratio. TIRF imaging is performed by illuminating the coverslip at the near critical angle, exciting fluorophores within a controlled range and minimizing the impact of out of focus light and background illumination. A 532-notch filter (OD>6, NF01-532U-25, Semrock) was used to reject light from the excitation source allowing several mirrors to reflect emitted light and the image passed through a Czerny-Turner monochromator (SP2150), which includes a blazed dispersive grating (150 groves/mm). The fluorescent image was finally passed through a matched tube lens and divided into a non-dispersed zeroth order image and a spectrally dispersed first-order image. By reflecting the zeroth order image to the output port using a silver mirror, the EM-CCD camera (proEM, Princeton Instruments) was used to capture the two images simultaneously. For this project the SPLM system has been modified; a schematic for the proposed system is shown FIG. 43a. The new optical design is more compact but retains the functionality of the original SPLM system.

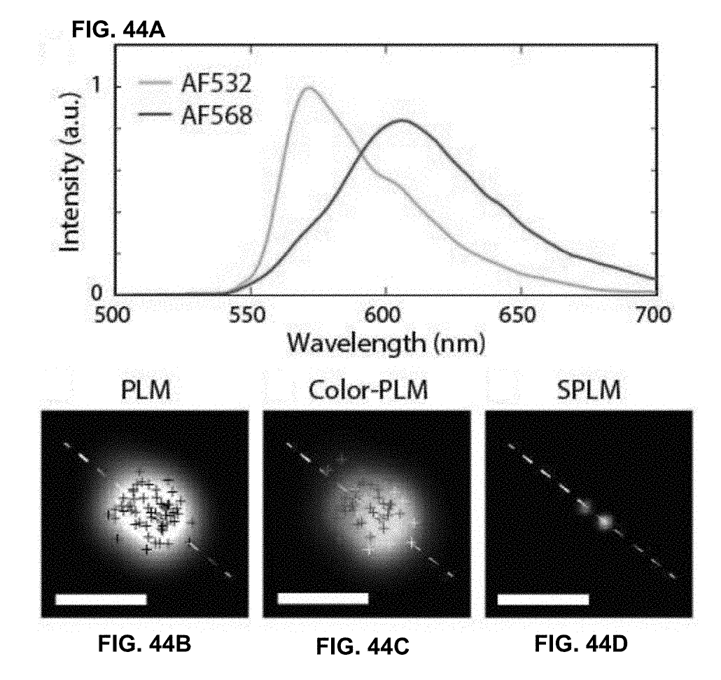

To test the functionality of the SPLM system, overlapping Alexa Fluor 532 and Alexa 568 fluorophores were captured. In this experiment, the excitation laser was used on high power (˜2 kWcm-2) to excite both fluorophores. Over time, the fluorophores in on-state undergo photobleaching and transition to dark state. A second laser with a wavelength of 405 nm was used in order to reactivate the fluorophores. The resulting stochastic emission as the transition from dark state to on-state was split using a ratio of 1:3. FIG. 43b demonstrates how each portion of the signal is detected using distinct sections of an EMCCD effectively coupling the emission events associated with the spectral and localization analysis. FIG. 44a and FIG. 44c show how the spectral signal from the two dyes can be captured and separated. While photon localization microscopy (PLM) depicted in FIG. 44b can be used to identify the incidence of emission it cannot be used to identify the emitter. By combining the localization and the spectroscopic signature data, distinct emitters can be identified with high spatial and spectral resolution as shown in FIG. 44d. The use of spectroscopy and photon localization has been validated by several groups; however previous attempts were limited by poor spectral resolution. The high spectral resolution achieved by our system allows us to use a wider range of dyes for imaging without adding multiple detectors. Further, by accurately sorting the emission for each fluorophore, overlapping emission from dyes could be identified and used to improve localization estimation and spatial resolution. The system was further tested using separate resolvable clusters of actin monomers labeled with Alexa Fluor 568. Spectral regression was used to identify changes between dye molecules emitting the same wavelength based on changes in their microenvironment. This practice adds increased ability to associate a distinct spectra with a single fluorophore and can be used to further improve the spatial resolution achieved by the SPLM system. The current system is able to achieve a Nyquist criterion spatial resolution of 10 nm and a spectral resolution of 0.63 nm. This SPLM has also been tested using DNA using novel emission strategies. This experience detecting DNA samples using SPLM will be play a vital role in improving imaging protocols for this project.

TB Extraction and Assay Development: The Center for Innovation in Global Health Technology (CIGHT) has developed a qPCR based assay for detection of TB using a specific capture-based DNA extraction strategy whole sputum. The current TB assay targets the senX3-regX3 and IS6110 genes specific to Mycobacterium tuberculosis complex species. The senX3-regX3 gene is present in many virulent forms of the bacteria. The IS6110 gene is less common in multiple types of TB but when expressed can be repeated at multiple sections of the genome. By using this multiplexed strategy, the sensitivity of detection of TB is improved. Additionally, CIGHT has also developed a protocol for specific capture of TB DNA from whole sputum. To begin the process, the sample is heated to 55° C. to thin the sputum and then to 95° C. to decontaminate the sample and denature the TB DNA. Once the DNA has been denatured, salt and biotinylated sequence-specific capture probes to senX3-regX3 and IS6110 are added to the sample and heated to 60° C. to allow hybridization of probes to DNA template to occur. Streptavidin-coated paramagnetic particles (PMP), with high affinity to biotin, are added to the sample. PMP-capture probe-DNA complexes are collected on the magnetic stand, and the sample is then washed to remove potential inhibitors in sputum. Finally, capture probes and PMPs are melted from the captured DNA by heating the sample to 75° C. CIGHT's extraction and PCR assay was tested in a blind study using 88 predicted pulmonary TB samples provided by the Foundation for Innovation in New Diagnostics (FIND). This study showed that the diagnostic had a high sensitivity (97%) between both culture positive/smear positive and culture positive/smear negative cases and high specificity (100%) in culture negative/smear negative sputum samples.

Probe selection: These strategies will be adapted for probes designed to target the katG and inhA genes of TB. KatG and inhA were selected due to their association with 76% of isoniazid resistant strains of TB. Isoniazid is a pro-drug which is activated by the TB enzyme catalase peroxidase; activation of isoniazid results in the production of free reactive radicals which damage the cell wall at specific targets. Mutations in katG account for 51% of all isoniazid drug resistance. A single base pair change from serine to threonine at 315 locus of the katG genome (Ser315Thr) accounts for 50%-90% of katG mutations. The Ser315Thr mutation is favorable since it has very little impact on the fitness of the bacteria. The presence of this mutation allows the bacteria to maintain normal levels catalase peroxidase production while decreasing the activity of isoniazid by reducing the stability of the enzyme. InhA TB gene is involved in cell wall mycolic acid synthesis and is one main target of the free radicals released by isoniazid. InhA promoter mutations feature an increase in inhA mRNA resulting in an overexpression of inhA and a reduction in isoniazid susceptibility. Point mutations in the inhA promoter have been identified as the major contributors to isoniazid resistance. The most common mutation occurs at position −15 in the promoter with mutations at positions −8 and −17 are also frequently reported. InhA mutations also contribute to resistance of the second-line drug ethionamide which has a similar structure and function to isoniazid. Probes targeting isoniazid resistance genes have be tested in past studies and have shown 83% sensitivity and 98% specificity.

Aim 1: Optimize SPLM platform for multiplex detection of low concentrations of synthetic wild-type DNA. Sample Preparation Protocol: To determine the ability of SPLM to detect TB, functionalized oligonucleotide probes specific to TB will be labeled with Alexa Fluor 568 and Alexa Fluor 532. A well-conserved sequence in the katG gene will be targeted and the Ser315Thr section of the katG gene, associated with isoniazid resistance, will also be targeted. By using probes for multiple targets, we hypothesize the probability of detecting DNA in low concentrations will increase. Further, by targeting drug resistance-associated mutations, this allows a pathway for detection of isoniazid resistance. To ensure binding, the probes will be exposed to denatured synthetic TB DNA sequences and the sample heated to 60° C. in the presence of salt. Once bound with probes, the DNA will be mounted on a silanized coverslip by pulling the treated coverslip through the DNA solution. Hydrophobic interactions between the coverslip and the DNA will cause the DNA to unfold, thus, increasing the distance between probes. Once the system is optimized for detecting katG probes, this method will be adapted for the inhA promoter probes labelled with additional dyes. By increasing the target sites, the probability of detection should increase. Additionally, targeting a second mutation demonstrates how the proposed method could be extended for the detection of multiple mutations which confer drug-resistance in a single sample.

Probe Design: Given the high resolution of the SPLM system, this presents the opportunity to test multiple targets within the selected genes. Using the 2223 base pair katG and the 289 base pair inhA promoter regions with lengths of 776 nm and 96 nm respectively, the optimum number of probes per gene, probe length and probe spacing can be tested. This information will be important in understanding the impact the proposed platform can have in identifying novel mutations. By using spectral regression and probe spacing below 10 nm, the highest resolution achievable by SPLM can be determined.

Imaging Protocol: Imaging will be performed using protocols and setup used in the preliminary study. During these experiments the spatial resolution will be improved by using spectral data from individual emitters within a cluster. Additionally, the temporal resolution can be improved by reducing the amount of emitted light allocated for spectral analysis. Since it may be less important to have high spectral resolution fewer pixels on the EMCCD can be used. This reallocation of efforts could instead be used to increase the signal to noise ratio as well as reduce image acquisition time.

Study Design: Once the SPLM system has been optimized experiments will be performed using varying concentrations of DNA. The limit of detection of the Xpert® MTB/RIF assay is 131 CFU/mL or 5 genomic copies of M. Tuberculosis. Typically 38 PCR cycles are required for detection using this assay. To compare the performance of SPLM to PCR, detection will be tested using samples with varying levels of amplification ranging from 0 to 40 with increasing increments of 5. The use of PCR will play an important role in standardizing the samples being tested by SPLM.

Image and Data Analysis: Images will be captured using Nikon software. Processing and analysis will be performed using the ImageJ plugin ThunderSTORM® software and additional analysis and processing will be done using custom MatLab scripts. The ThunderSTORM® software will be used to process localization of the stochastic emission events. The spectral data will be processed using custom scripts will be combined with the localization information. Together this data will be compiled and signals which exceed established thresholds will be used to indicate the presence of TB DNA.

Statistical Analysis: Thresholds for binding at each section will be set using log likelihood test for alpha (false positive) values of 0.05 and a power (probability of detection) of 0.95. Power analysis will be performed to determine the number of samples required for testing changes in the detected signals. The result of the power analysis will also be used to determine the minimum number of repetitions required for each sample. Additionally, the detected signals using PCR and SPLM will be compared. It is expected that the detectable signal recorded by the SPLM system will be significantly higher than signals for PCR at low amplification levels.

Anticipated Problems and Alternative Approaches: Hybridization of DNA may prove to be a challenge since hybridization times can vary based on probe length, salt concentration and hybridization buffer. To assess the performance of the new probes senX3-regX3 probes will also be used as a control to measure the performance of the katG and inhA probes. Another major challenge will be the transfer of the hybridized sample to a coverslip for imaging. The use of silanized coverslip is preferable since it does not require modified sample chambers and limits the interactions of the DNA of interest with other molecules. However, if the proposed mounting method proves to have a high impact on the detection, the DNA could be mounted on a functionalized coverslip using streptavidin and biotin tethers. For this method a modified sample chamber will be developed to allow the DNA to be flowed over the coverslip allowing the DNA to be stretched during detection.

Expected Results: The SPLM system should be able to detect signals from all targets for wild-type TB. The SPLM system is expected to detect signals which exceed the established threshold for unamplified samples and amplifications below 20 cycles. Further, PCR detection of equivalent samples should be below the established thresholds.

Aim 2: Test the performance of the system for the detection of TB and different types of isoniazid resistant TB. Sample Preparation: Synthetic DNA associated with wild-type TB, katG mutations, inhA promoter mutations and DNA associated with mutations in both katG and inhA will be tested. Optimized sample preparation methods described in aim 1 will be repeated.

Study Design—Probe Testing: The foundation of the SPL-Gene Testing platform will be the differences in probe affinity for mutants. To ensure the optimum probe design is being used several possible changes at the targeted locus associated with resistance will be tested. Using wild-type DNA as the reference for probe binding novel sequences will be tested to ensure high specificity. Detection of mutants using PCR will also be performed using the amplification levels and replicates from aim 1.

Imaging Protocol and Analysis: Strategies developed for optimized imaging and analysis from aim 1 will be used for this step. The limit of detection (LOD) of the system will be determined using the data from this step. Software will be developed to categorize the detected strain. Further, to improve the ease of use of the SPLM system an add-on will be designed to display the results of each test. This software will serve as the basis for a streamlined SPL-Gene Testing diagnostic.

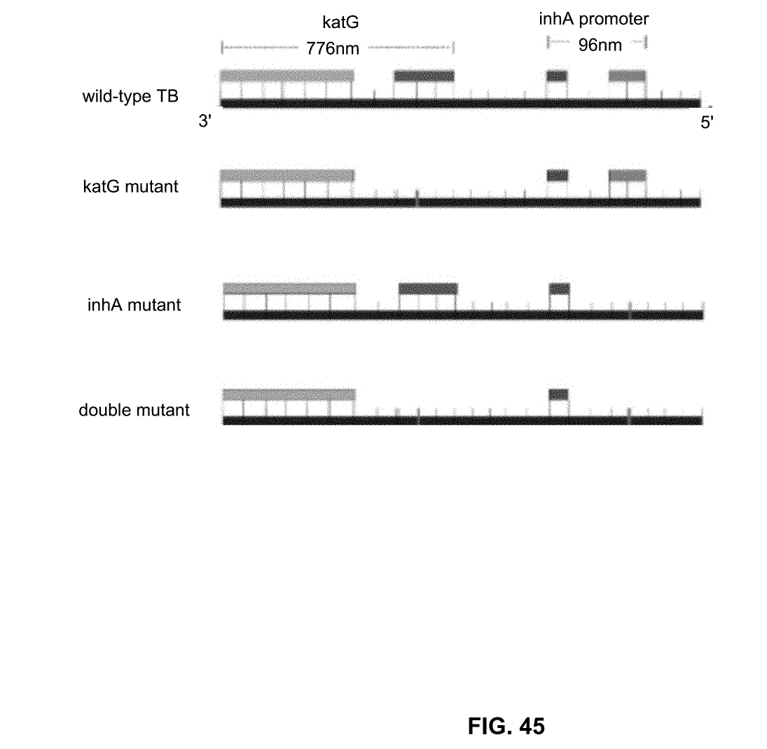

Statistical Analysis: A two way Anova will be performed to determine the whether the changes in the signal are statistically different. It is expected that probes associated with conservative segments of the TB genome will be above the threshold and sections associated with drug resistance will be below the threshold. These results are shown in FIG. 45 where mutations indicated in pink prevent probes from binding, resulting in a reduction in the detected signals.

Anticipated Problems and Alternative Approaches: Non-specific binding of the probes associated with mutations could occur making detection difficult. The length of the probes and the hybridization temperature will be adjusted to reduce this effect.

Expected Results: Synthetic DNA for wild-type TB and specific mutations will be detected and characterized with high specificity and sensitivity. Similar to results from aim 1, SPLM is expected to detect signals in at low amplification levels (below 20 cycles) while detection by PCR should fail.

Aim 3: Test the performance of the probes and platform for TB detection in complex sample types. Study Design: This aim will be performed using the protocols and methods developed during aim 1 and 2. The optimized sample preparation, probe design and imaging protocol will be used to detect synthetic wild-type TB DNA, katG mutant TB DNA, inhA mutant TB DNA and combined katG and inhA mutant TB DNA in complex sample types. The first test will be detection in the presence of other genomic forms such as sperm whale DNA. The purpose of this test is to perform rigorous specificity testing. The next test will be performed by spiking the synthetic DNA into sputum and testing detection following extraction. The extraction methods used in this step have been discussed in the preliminary study section. These complex sample types will be repeated using genomic TB DNA. The final step will be to perform LOD testing using genomic DNA and the extraction assay. Detection data using the extraction assay and PCR using the amplification protocol from aim 1 will also be collected. As a control synthetic wild-type DNA will be used as a reference for image analysis. Additionally, an estimate of the time required for sample preparation and detection will be determined. The time for each step will be used to identify limiting steps in the detection workflow. Additionally, areas where automation can be used to decrease processing time will be identified.

Imaging Protocol and Sample Preparation: The imaging protocols and sample preparation developed in aims 1 and 2 will be used to complete this portion of the project.

Statistical Analysis: A two way Anova test will be used to compare the results from the complex samples to the reference sample. The ability of SPLM to detect the complex samples will also be compared to detection using PCR at equivalent amplification levels.

Anticipated Problems and Alternative Approaches: The major problems at this stage would be reduction in hybridization times. To improve hybridization minor modifications to the salt concentration. Complex samples have the potential to reduce detectability of the dyes due to increased background signals. This will have a significant impact on the LOD determined in aim 2. To address this avenues for improving the extraction assay will be explored. Additionally, algorithms for noise reduction and improved processing of the spectral signal could be employed.

Expected Results: A reduction in detection is expected when TB DNA in complex sample types. However, the use of the extraction assay is expected to remove excess particles from the sample prior to hybridization. Therefore, the LOD of the system should be comparable to the LOD measured in aim 2. At this point, the detection system, extraction and sample processing protocols can be further assessed and validated using clinical TB samples provided by FIND.

### V. Software and Computer Systems for Spectroscopic Super-resolution Microscopic Imaging

In various examples, certain methods and systems may further include software programs on computer systems and use thereof. Accordingly, computerized control for the synchronization of system functions such as laser system operation, fluid control function, and/or data acquisition steps are within the bounds of the invention. The computer systems may be programmed to control the timing and coordination of delivery of sample to a detection system, and to control mechanisms for diverting selected samples into a different flow path. In some examples, the computer may also be programmed to store the data received from a detection system and/or process the data for subsequent analysis and display.

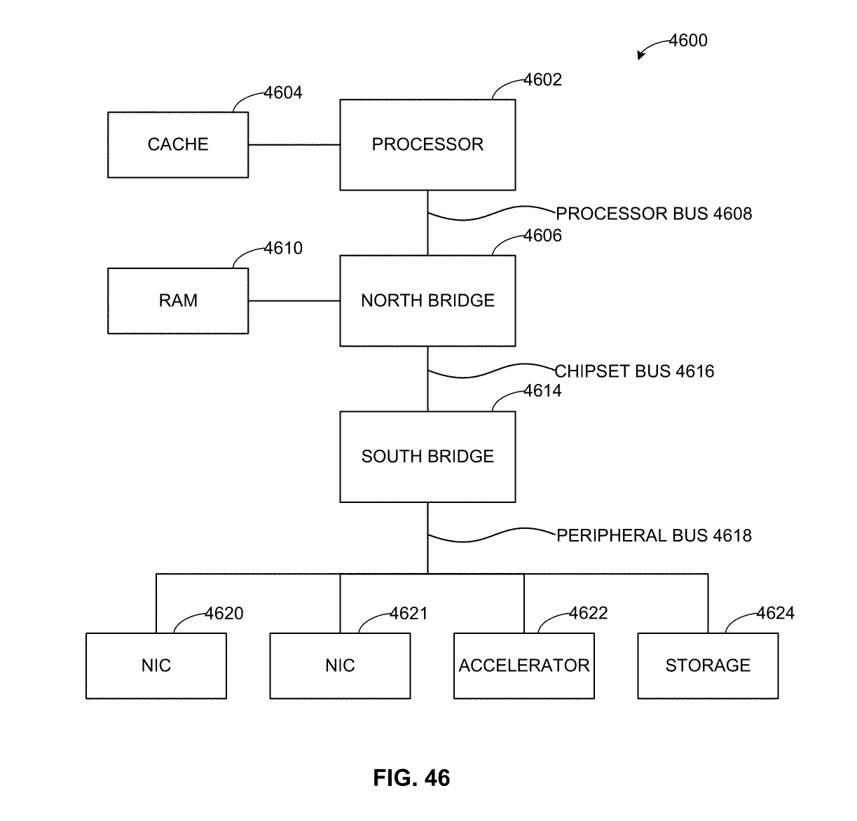

FIG. 46 is a block diagram illustrating a first example architecture of a computer system 4600 that can be used in connection with examples disclosed and described herein. As depicted in FIG. 46, the example computer system can include a processor 4602 for processing instructions. Non-limiting examples of processors include: Intel Xeon™ processor, AMD Opteron™ processor, Samsung 32-bit RISC ARM 1176JZ(F)-S vl .O™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some examples, multiple processors or processors with multiple cores can also be used, whether in a single computer system, in a cluster, or distributed across systems over a network comprising a plurality of computers, cell phones, and/or personal data assistant devices.

As illustrated in FIG. 46, a high speed cache 4604 can be connected to, or incorporated in, the processor 4602 to provide a high speed memory for instructions or data that have been recently, or are frequently, used by processor 4602. The processor 4602 is connected to a north bridge 4606 by a processor bus 4608. The north bridge 4606 is connected to random access memory (RAM) 4610 by a memory bus 4612 and manages access to the RAM 4610 by the processor 4602. The north bridge 4606 is also connected to a south bridge 4614 by a chipset bus 4616. The south bridge 4614 is, in turn, connected to a peripheral bus 4618. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 4618. In some alternative architectures, the functionality of the north bridge can be incorporated into the processor instead of using a separate north bridge chip.

In some examples, system 4600 can include an accelerator card 4622 attached to the peripheral bus 4618. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 4624 and can be loaded into RAM 4610 and/or cache 4604 for use by the processor. The system 4600 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with certain examples.

In this example, system 4600 also includes network interface cards (NICs) 4620 and 4621 connected to the peripheral bus for providing network interfaces to external storage, such as Network Attached Storage (NAS) and other computer systems that can be used for distributed parallel processing.

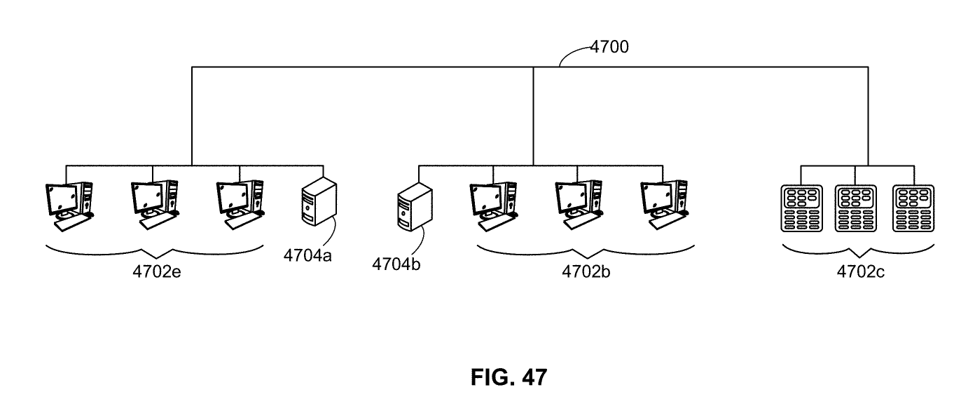

FIG. 47 is a diagram showing a network 4700 with a plurality of computer systems 4702a, and 4702b, a plurality of cell phones and personal data assistants 4702c, and Network Attached Storage (NAS) 4704a, and 4704b. In some examples, systems 4702a, 4702b, and 4702e can manage data storage and optimize data access for data stored in Network Attached Storage (NAS) 4704a and 4704b. A mathematical model can be used for the data and be evaluated using distributed parallel processing across computer systems 4702a, and 4702b, and cell phone and personal data assistant systems 4702c. Computer systems 4702a, and 4702b, and cell phone and personal data assistant systems 4702c can also provide parallel processing for adaptive data restructuring of the data stored in Network Attached Storage (NAS) 4704a and 4704b. FIG. 47 illustrates an example only, and a wide variety of other computer architectures and systems can be used in conjunction with the various examples of the present invention. For example, a blade server can be used to provide parallel processing. Processor blades can be connected through a back plane to provide parallel processing. Storage can also be connected to the back plane or as Network Attached Storage (NAS) through a separate network interface.

In some example examples, processors can maintain separate memory spaces and transmit data through network interfaces, back plane or other connectors for parallel processing by other processors. In other examples, some or all of the processors can use a shared virtual address memory space.

The above computer architectures and systems are examples only, and a wide variety of other computer, cell phone, and personal data assistant architectures and systems can be used in connection with example examples, including systems using any combination of general processors, co-processors, FPGAs and other programmable logic devices, system on chips (SOCs), application specific integrated circuits (ASICs), and other processing and logic elements. In some examples, all or part of the computer system can be implemented in software or hardware. Any variety of data storage media can be used in connection with example examples, including random access memory, hard drives, flash memory, tape drives, disk arrays, Network Attached Storage (NAS) and other local or distributed data storage devices and systems.

In some examples of present disclosure, the computer system can be implemented using software modules executing on any of the above or other computer architectures and systems. In other examples, the functions of the system can be implemented partially or completely in firmware, programmable logic devices such as field programmable gate arrays, system on chips (SOCs), application specific integrated circuits (ASICs), or other processing and logic elements.

What is claimed is:

1. A method for identifying one or more cellular features, the method comprising:
a) providing a test sample obtained from a subject, wherein the test sample contains one or more cellular derived elements;
b) activating a subset of light-emitting molecules in the one or more cellular derived elements in a wide-field area of the test sample, using an excitation light;
c) capturing one or more images of the light emitted from the subset of the molecules illuminated with the excitation light;
d) localizing one or more activated light emitting molecules, using one or more single molecule microscopic methods to obtain localization information;
e) simultaneously capturing spectral information for the same localized activated light emitting molecules using the one or more spectroscopic methods;
f) resolving one or more non-diffraction limited images of the one or more cellular derived elements using a combination of the localization and spectral information for the localized activated light emitting molecules;
g) displaying the one or more non-diffraction limited images;
h) identifying one or more cellular elements by comparing the one or more non-diffraction limited images of one or more features to a reference.

2. A system configured for identifying one or more cellular features in a cell, the system comprising:
a) obtaining a test sample obtained from a subject, wherein the test sample contains one or more cellular derived elements available for imaging;
b) a device configured to activate a subset of light-emitting molecules in the one or more cellular derived elements in a wide-field area of the test sample, using an excitation light;
c) a device configured to capture one or more images of the light emitted from the subset of the molecules illuminated with the excitation light;
d) a device configured to localize one or more activated light emitting molecules, using one or more single molecule microscopic methods to obtain localization information;
e) a device and computer-readable program code configured to simultaneously capture spectral information for the same localized activated light emitting molecules using the one or more spectroscopic methods;
f) a device and computer-readable program code configured to resolve one or more non-diffraction limited images of the one or more cellular derived elements using a combination of the localization and spectral information for the localized activated light emitting molecules;
g) a device and computer-readable program code configured to display the one or more non-diffraction limited images; and
h) a computer-readable program code configured to identify one or more features in the cellular derived elements by comparing the one or more non-diffraction limited images of one or more genetic features to a reference.

3. The method of claim 1, wherein the method comprises assessing the presence or absence of a disease in the subject based on identifying the one or more genetic features in the cellular derived elements by comparing the one or more non-diffraction limited images of one or more genetic features to a reference.

4. The method of claim 1, wherein the method comprises assessing one or more characteristics of a disease in the subject based on identifying the one or more genetic features in the cellular derived elements by comparing the one or more non-diffraction limited images of one or more genetic features to a reference.

5. The method of claim 4, wherein the one or more characteristics of the disease comprises disease progression, disease stage, cancer stage, disease classification, chronic disease, acute disease, deficiency disease, hereditary disease, physiological disease, karyotype, morbidity of the subject, and survival time of the subject.

6. The method of claim 1, wherein the test sample comprises cells, fixed cells, live cells, smear test, fine-needle aspiration, biopsy, cytological specimen, resected specimen, cytological brushing specimen, a formaldehyde fixed paraffin embedded tissue, pap smear, buccal swab, colon swab, mucus sample, urine, blood, bodily fluid sample, cell culture and tissue sample.

7. The method of claim 1, wherein the test sample comprises cells, lysed cells, organelles, cellular membranes, purified chromosomes, partial chromosomes, chromatin, genomes, partial genomes, or chromosome spreads, nucleic acids, proteins, carbohydrates, or lipids.

8. The method of claim 1, wherein the feature comprises a mutation, chromosomal aberration, chromosomal alteration, nucleosome distance, chromosomal integrity, epigenetic markers, transcriptional activity, genetic translocation, copy number variation, gene duplication, genetic rearrangements, sequence localization, genetic deletions, tandem duplications, inversions, insertions, mobile element insertions, aneuploidy, polyploidy, polymorphisms, chromosomal amplification, homozygosity or heterozygosity.

9. The method of claim 1, wherein the identifying one or more features comprises contacting the cellular derived elements with one or more labels comprising a fluorescence in situ hybridization probe, in situ hybridization probe, unlabeled probe, labeled probe, unlabeled nucleic acid, labeled nucleic acid, comparative genomic hybridization probes, singe nucleotide polymorphism array probes, labeled chromatin antibodies, fluorescent dyes, dyes or stains, antibody or ligand.

10. The method of claim 1, wherein the identifying one or more features uses label free imaging of the one or more cellular derived elements.

11. The method of claim 1, wherein the reference comprises a normal cell, healthy cell, normal chromosome, sample containing one or more normal elements, sample containing one or more wild-type elements, sample derived from healthy tissue, sample derived from a healthy area of the test sample, a reference threshold, a reference level, a reference localization pattern, a reference fingerprint, a reference molecular profile or a reference copy number.

12. The method of claim 1, the method comprising detecting the presence or absence of a desired feature obtained by genetic manipulation in a live cell, wherein the genetic manipulation comprises site directed mutatgenesis, mutagenesis, homologous recombination, genet targeting, use of restriction endonucleases, use of nucleases, use of ligation enzymes, use of clustered regularly-interspaced short palindromic repeats (CRISPR) enzymes, use of recombination, use of homing endonucleases, use of transcription activator-life effector nucleases, use of zinc-finger nucleases, transformation, transfection, viral mediated nucleic acid integration, transposable elements, or mobile elements, inducement of stem cell characteristics, inducement of pluripotency or differentiation, engineering for antibody production, engineering for artificial production of a transgene.

13. The method of claim 1, the method comprising providing a treatment to the subject based on identifying one or more characteristics of the pathogens or suspected pathogens by comparing the molecular profile generated from the one or more non-diffraction limited images to a reference.

14. The method of claim 1, the method further comprising stratifying one or more treatment options provided to the subject based on the identifying one or more characteristics of the pathogens or suspected pathogens by comparing the molecular profile generated from the one or more non-diffraction limited images to a reference.
